SEQUENCE LISTING (26 sequences)

SEQ ID NO: 1 - protein, 9 aa, Artificial
  Synthetic
GGSGGSGGS

SEQ ID NO: 2 - protein, 30 aa, Artificial
  Synthetic
GCCTGTCTTTGCCTGTCTTTGCCTGTCTTT

SEQ ID NO: 3 - protein, 8 aa, Artificial
  Synthetic
GTGTCCCC

SEQ ID NO: 4 - protein, 5 aa, Artificial
  Synthetic
APRPG

SEQ ID NO: 5 - protein, 8 aa, Artificial
  Synthetic
GPLGIAGQ

SEQ ID NO: 6 - protein, 5 aa, Artificial
  Synthetic
RGBYC

SEQ ID NO: 7 - protein, 6 aa, Artificial
  Synthetic
KCCYSL

SEQ ID NO: 8 - protein, 7 aa, Artificial
  Synthetic
YEVGHRC

SEQ ID NO: 9 - DNA, 31 bp, Artificial
  Synthetic
ccccttcagtctggaaaggaggacactgagg

SEQ ID NO: 10 - DNA, 31 bp, Artificial
  Synthetic
ccccttcagtatggaaaggaggacactgagg

SEQ ID NO: 11 - DNA, 31 bp, Artificial
  Synthetic
atcagtccagccccttcagtctggaaaggag

SEQ ID NO: 12 - DNA, 31 bp, Artificial
  Synthetic
atcagtccagccccttcagtatggaaaggag

SEQ ID NO: 13 - DNA, 36 bp, Artificial
  Synthetic
aatcagtccagccccttcagtctggaaaggaggaca

SEQ ID NO: 14 - DNA, 37 bp, Artificial
  Synthetic
aatcagtccagccccttcaagtctggaaaggaggaca

SEQ ID NO: 15 - DNA, 37 bp, Artificial
  Synthetic
aatcagtccagccccttcacgtctggaaaggaggaca

SEQ ID NO: 16 - DNA, 34 bp, Artificial
  Synthetic
caatcagtccagccccttcagtctggaaaggagg

SEQ ID NO: 17 - DNA, 37 bp, Artificial
  Synthetic
caatcagtccagccccttcacgggtctggaaaggagg

SEQ ID NO: 18 - DNA, 35 bp, Artificial
  Synthetic
caatcagtccagccccttcaagtctggaaaggagg

SEQ ID NO: 19 - DNA, 30 bp, Artificial
  Synthetic
cagtccagccccttcagtctggaaaggagg

SEQ ID NO: 20 - DNA, 31 bp, Artificial
  Synthetic
tcagtccagccccttcagtctggaaaggagg

SEQ ID NO: 21 - DNA, 32 bp, Artificial
  Synthetic
tcagtccagccccttcaagtctggaaaggagg

SEQ ID NO: 22 - DNA, 30 bp, Artificial
  Synthetic
tcagtccagccccttcagtctggaaaggag

SEQ ID NO: 23 - DNA, 31 bp, Artificial
  Synthetic
tcagtccagccccttcaagtctggaaaggag

SEQ ID NO: 24 - DNA, 31 bp, Artificial
  Synthetic
tcagtccagcccctttcagtctggaaaggag

SEQ ID NO: 25 - DNA, 31 bp, Artificial
  Synthetic
tcagtccagccccttcaggtctggaaaggag

SEQ ID NO: 26 - DNA, 31 bp, Artificial
  Synthetic
tcagtccagccccttcatgtctggaaaggag

(19) United States
(12) Patent Application Publication
(10) Pub. No.: US 2022/0389451 A1
(43) Pub. Date: Dec. 8, 2022

(54) COMPOSITIONS AND METHODS FOR DELIVERING CARGO TO A TARGET CELL

(71) Applicants: THE BROAD INSTITUTE, INC., Cambridge, MA (US); MASSACHUSETTS INSTITUTE OF TECHNOLOGY, Cambridge, MA (US)

(72) Inventors: Feng Zhang, Cambridge, MA (US); Michael Segel, Cambridge, MA (US)

(73) Assignees: THE BROAD INSTITUTE, INC., Cambridge, MA (US); MASSACHUSETTS INSTITUTE OF TECHNOLOGY, Cambridge, MA (US)

(21) Appl. No.: 17/761,641

(22) PCT Filed: Sep. 18, 2020
(86) PCT No.: PCT/US2020/051637

§ 371 Date: Mar. 18, 2022

Related U.S. Application Data
(60) Provisional application No. 62/903,127, filed on Sep. 20, 2019.
(60) Provisional application No. 63/003,409, filed on Apr. 1, 2020.

(51) Int. Cl. C12N 15/86; C07K 14/005

(52) U.S. Cl. CPC C12N 15/86; C07K 14/005; C07K 2319/735; C12N 2740/10023; C12N 2740/10042; C12N 2740/10022; C12N 2740/13023; C12N 2740/13042; C12N 2740/14022; C12N 2740/14023; C12N 2740/14042

(57) ABSTRACT

Provided herein are compositions, systems, and methods for delivering cargo to a target cell. The compositions, systems, and methods comprise one or more polynucleotides encoding one or more endogenous retroviral elements for forming a delivery vesicle and one or more capture moieties for packaging a cargo within the delivery vesicle. The one or more endogenous retroviral elements for forming a delivery vesicle may comprise two or more of a retroviral gag protein, a retroviral envelope protein, a retroviral reverse transcriptase or a combination thereof. The retroviral gag protein alone, the retroviral envelope protein alone, or both the retroviral gag protein and retroviral envelope protein may be endogenous.

### CROSS-REFERENCE TO RELATED APPLICATIONS

This application claims the benefit of U.S. Provisional Application No. 62/903,127, filed Sep. 20, 2019 and U.S. Provisional Application No. 63/003,409, filed Apr. 1, 2020. The entire contents of the above-identified applications are hereby fully incorporated herein by reference.

### STATEMENT REGARDING FEDERALLY SPONSORED RESEARCH

This invention was made with government support under Grant No. HL141201 awarded by the National Institutes of Health. The government has certain rights in the invention.

### REFERENCE TO AN ELECTRONIC SEQUENCE LISTING

The contents of the electronic sequence listing (“BROD-4620 WP_ST.25.txt,” size is 4,945 bytes and it was created on Sep. 18, 2020 is herein incorporated by reference in its entirety.

### TECHNICAL FIELD

The subject matter disclosed herein is generally directed to engineered delivery agents, compositions, systems and uses thereof.

### BACKGROUND

Delivery systems are important aspects to efficacy of a treatment. Delivery of therapeutics to the inside of a cell presents many challenges, including but not limited to, limiting off-target effects, delivery efficiency, degradation, and the like. Viruses and virus-like particles have been used to deliver various cargos (e.g. gene therapy agents) to target cells. However, currently used vesicles and particles may be large in size and difficult to generate in a consistent manner. As such, there exists a need for simpler and improved delivery systems.

### SUMMARY

In certain example embodiments, the invention provides an engineered delivery system comprising one or more polynucleotides, wherein the one or more polynucleotides encodes one or more endogenous retroviral elements for forming a delivery vesicle and one or more capture moieties for packaging a cargo within the delivery vesicle.

In some embodiments, the one or more endogenous retroviral elements for forming a delivery vesicle comprises two or more of a retroviral gag protein, a retroviral envelope protein, a retroviral reverse transcriptase or a combination thereof.

In some embodiments, the retroviral gag protein may be endogenous. In some embodiments, the retroviral envelope protein may be endogenous. In some embodiments, the retroviral gag protein and the retroviral envelope protein are both endogenous.

In some embodiments, the retroviral gag protein contains the NC and MA domains.

In some embodiments, the retroviral gag protein is a gag-homology protein. In some embodiments, the gag-homology protein is Arc1, Asprv1, PNMA1, PNMA3, PNMA4, PNMA5, PNMA6, PNMA7, PEG10, RTL1, MOAP1, or ZCCHC12. In specific embodiments, the gag-homology protein is PNMA4, PEG10, or RTL1.

In some embodiments, the envelope protein may be from a Gammaretrovirus or a Deltaretrovirus. In some embodiments, the envelope protein is selected from envH1, envH2, envH3, envK1, envK2 1, envK2 2, envK3, envK4, envK5, envK6, envT, envW, envW1, envfrd, envR(b), envR, envF(c)2, or envF(c)1.

In some embodiments, the envelope protein comprises a cargo-binding domain. In some embodiments, the cargo-binding domain is a hairpin loop-binding element. In some embodiments, the hairpin loop-binding element is an MS2 aptamer.

In some embodiments, the delivery system elicits a poor immune response.

In some embodiments, the cargo comprises nucleic acids, proteins, a complex thereof, or a combination thereof. In some embodiments, the cargo is linked to one or more envelope proteins by a linker. In some embodiments, the linker is a glycine-serine linker. In some embodiments, the glycine-serine linker is (GGS)3 (SEQ ID NO: 1).

In some embodiments, the cargo comprises a ribonucleoprotein. In some embodiments, the cargo comprises a genetic modulating agent. In some embodiments, the genetic modulating agent comprises one or more components of a gene editing system and/or polynucleotides encoding thereof. In some embodiments, the gene editing system is a CRISPR-Cas system. In some embodiments, the CRISPR-Cas system is a Type II, Type V, or Type VI CRISPR-Cas system. In some embodiments, the Type II CRISPR-Cas system comprises CRISPR-Cas9. In some embodiments, the Type V CRISPR-Cas system comprises CRISPR-Cas12. In some embodiments, the Type VI CRISPR-Cas system comprises CRISPR-Cas13.

In some embodiments, a Cas protein of the CRISPR-Cas system may be modified to bind to a binding domain of the envelope protein. In some embodiments, a guide molecule of the CRISPR-Cas system is modified to bind to a binding domain of the envelope protein. In some embodiments, the modification comprises incorporation of a hairpin loop that binds to a hairpin-binding element on the envelope protein. In some embodiments, the hairpin loop may be recognized by the MS2 aptamer.

In some embodiments, the system may further comprise a reverse transcriptase.

In some embodiments, the one or more capture moieties comprise DNA-binding moieties, RNA-binding moieties, protein-binding moieties, or a combination thereof.

In some embodiments, the delivery vesicle is a virus-like particle.

In some embodiments, the system may further comprise a targeting moiety, wherein the targeting moiety is capable of specifically binding to a target cell. In some embodiments, the targeting moiety comprises a membrane fusion protein. In some embodiments, the membrane fusion protein is the G envelope protein of vesicular stomatitis virus (VSV-G).

In some embodiments, the target cell is a mammalian cell. In some embodiments, the mammalian cell is a cancer cell. In some embodiments, the mammalian cell is infected with a pathogen. In some embodiments, the pathogen is a virus.

In another aspect, the invention provides a delivery vesicle comprising one or more components encoded in the one or more polynucleotides in the engineered delivery system described herein.

In some embodiments, the one or more components of the delivery vesicle comprise two or more of a retroviral gag protein, a retroviral envelope protein, a retroviral reverse transcriptase, or a combination thereof.

In some embodiments, the retroviral gag protein is a gag-homology protein selected from the group consisting of Arc1, Asprv1, PNMA1, PNMA3, PNMA4, PNMA5, PNMA6, PNMA7, PEG10, RTL1, MOAP1, or ZCCHC12. In specific embodiments, the gag-homology protein is PNMA4, PEG10, or RTL1.

In some embodiments, the vesicle comprises a cell-specific targeting moiety. In some embodiments, the cell-specific targeting moiety targets a mammalian cell. In some embodiments, the cell-specific targeting moiety comprises a membrane fusion protein. In some embodiments, the membrane fusion protein is VSV-G.

In some embodiments, the mammalian cell is a cancer cell. In some embodiments, the mammalian cell is infected with a pathogen. In some embodiments, the pathogen is a virus.

In yet another aspect, the invention provides a system for delivering a cargo to a target cell, comprising a delivery vesicle enclosing a cargo and an endogenous reverse transcriptase.

In some embodiments, the delivery vesicle is a virus-like particle. In some embodiments, the delivery vesicle is comprised of a retroviral gag protein and a retroviral envelope protein. In some embodiments, the retroviral gag protein originates from human endogenous retroviruses (HERVs).

In some embodiments, the retroviral gag protein is Arc1, Asprv1, PNMA1, PNMA3, PNMA4, PNMA5, PNMA6, PNMA7, PEG10, RTL1, MOAP1, or ZCCHC12. In specific embodiments, the retroviral gag protein is PNMA4, PEG10, or RTL1.

In some embodiments, the retroviral envelope protein originates from HERVs. In some embodiments, the retroviral gag protein and the retroviral envelope protein both originate from HERVs.

In some embodiments, the retroviral envelope protein comprises a cargo-binding domain. In some embodiments, the cargo-binding domain is a hairpin loop-binding element. In some embodiments, the hairpin loop-binding element is an MS aptamer.

In some embodiments, the cargo comprises nucleic acids, proteins, a complex thereof, or a combination thereof. In some embodiments, the cargo comprises a ribonucleoprotein. In some embodiments, the cargo comprises a genetic modulating agent. In some embodiments, the genetic modulating agent comprises one or more components of a gene editing system and/or polynucleotides encoding thereof. In some embodiments, the gene editing system is a CRISPR-Cas system. In some embodiments, the CRISPR-Cas system is a Type II, Type V, or Type VI CRISPR-Cas system. In some embodiments, the Type II CRISPR-Cas system comprises CRISPR-Cas9. In some embodiments, the Type V CRISPR-Cas system comprises CRISPR-Cas12. In some embodiments, the Type VI CRISPR-Cas system comprises CRISPR-Cas13.

In some embodiments, the cargo is linked to one or more envelope proteins by a linker.

In some embodiments, the linker is a glycine-serine linker. In some embodiments, the glycine-serine linker is (GGS)₃ (SEQ ID NO: 1).

In some embodiments, a Cas protein of the CRISPR-Cas system is modified to bind to a binding domain of the envelope protein. In some embodiments, a guide molecule of the CRISPR-Cas system is modified to bind to a binding domain of the envelope protein. In some embodiments, the modification comprises incorporation of a hairpin loop that binds to a hairpin-binding element on the envelope protein. In some embodiments, the hairpin loop is recognized by the MS2 aptamer.

In some embodiments, the system may further comprise a membrane fusion protein. In some embodiments, the membrane fusion protein is VSV-G.

In some embodiments, the target cell is a mammalian cell. In some embodiments, the mammalian cell is a cancer cell. In some embodiments, the mammalian cell is infected with a pathogen. In some embodiments, the pathogen is a virus.

In yet another aspect, the invention provides a method for treating a disease, comprising administering any of the systems described herein to a subject in need thereof, wherein the delivery vesicle delivers the cargo to one or more cells of the subject.

In some embodiments, the cargo may comprise a therapeutic agent. In some embodiments, the therapeutic agent comprises one or more components of a gene editing system and/or polynucleotide encoding thereof.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—shows expression of various env proteins in HEK293T cells, with increased expression shown for Envw1, Envk1, and Envfrd. p FIG. 2—shows expression of various endogenous retroviral glycoproteins from particles that are pseudotyped with lentiviral proteins.

FIG. 3—shows expression of a Pnma3-RFP fusion construct (illustrated at the top) compared to a lentivirus-RFP reporter in mouse neuronal cells. Micrographs show organotypic culture slices from the prefrontal cortex.

FIG. 4—shows maps of various endogenous gag proteins tested for their ability to form capsids, secrete proteins, and transfer materials to a new cell.

FIG. 5—images of transmission electron micrographs showing the ability of various endogenous gag protein candidates to form capsids.

FIG. 6—shows ability of various endogenous gag proteins to be secreted from cells.

FIGS. 7A, 7B—shows gag constructs containing Cas9/gRNA complexes in the absence (7A) and presence (7B) of membrane fusion protein VSV-G.

FIG. 8—schematic illustrating the experimental outline.

FIGS. 9A, 9B—alignment of sequences showing the number of mutations introduced with CRISPR complexes transferred in vesicles comprising RTL1 (9B) versus control vesicles (9A).

FIG. 10—graph showing the of number indels induced by editing complexes in vesicles comprising various gag-homology proteins.

FIGS. 11A-11C—illustrate the ability of (11A) PNMA4, (11B) PEG10, and (11C) RTL1 to transfer Cas9/gRNA complexes to a new cell.

FIG. 12—alignment of sequences of knock-in mice that expressed an HA-tag on endogenous RTL-1.

FIG. 13—nitrocellulose gel showing HA-tagged PEG10 and RTL1.

FIGS. 14A-14D—immunofluorescence images illustrating the ability of various gag-homology proteins (14B-14D) to form vesicles in the presence of VSV-G compared to control particles (14A).

FIGS. 15A, 15B—graphs showing copy numbers of vesicles produced in the presence of various gag-homology proteins.

FIG. 16—graph showing fold change in viral infectivity when various gag-homology proteins are overexpressed.

FIG. 17—schematic showing various putative endogenous signaling systems on a scale of decreasing immunogenicity.

FIG. 18—schematic showing the requirements for an enveloped VLP.

FIG. 19—electron micrographs showing the ability of various gag-homology proteins to spontaneously form vesicles from cells.

FIG. 20—electron micrographs showing the ability of various gag-homology proteins to spontaneously form vesicles from cells.

FIG. 21—immunoprecipitation assays showing various gag-homology proteins secreted from cells.

FIG. 22—schematic showing an assay used for determining whether GAGs are taken up by cells.

FIGS. 23A-23D—(23A, 23B) show the ability of various gag constructs to be taken up by cells and introduce indels into target sequences; (23A) SEQ ID NO:9-18; (23B) SEQ ID NO:19-26; (23C, 23D) graphs showing the ability of vesicles to be taken up into HEK293FT cells in the (23C) absence and (23D) presence of VSV-G.

FIG. 24—immunoprecipitation assay showing the ability of various constructs to be taken up by cells in the absence (left) and presence (right) of VSV-G.

FIG. 25—schematic showing the two overlapping reading frames of PEG10.

FIG. 26—immunoprecipitation gel showing bands for both translated ORF1 and ORF1/2 of PEG10.

FIG. 27—immunoprecipitation reactions from whole cell lysates of cells transfected with various PEG10 constructs.

FIG. 28—immunoprecipitation reactions from whole cell lysates and VLP fractions of cells transfected with various PEG10 constructs.

FIG. 29—immunoprecipitation assay analyzing the ability of VSV-G and SGCE to boost PEG10 secretion and uptake into target cells.

FIG. 30—immunoprecipitation gels showing the ability of sucrose cushions of various concentrations to boost the delivery efficiency of PEG10.

FIG. 31—graph showing percent INDEL generation by use of various constructs.

FIG. 32—Western blots and immunofluorescent stains slowing the location of PEG10 in both the serum and cortex neurons in the brain.

FIG. 33—graph showing that knockout mice lacking PEG10 show early embryonic lethality, indicating the importance of this gene in embryonic development.

FIG. 34—RNA-seq gene ontology analysis of primary mouse neurons revealed three groups of differentially expressed genes: 1) genes involved in chromatin remodeling; 2) genes involved in the trans-golgi network and exocytosis, and 3) SNAREs and other genes coding for endosomal proteins.

FIG. 35—fluorescent micrographs showing expression of GFP/PEG10 reporter constructs.

FIG. 36—schematic showing a DNA methyltransferase identification mechanism (DamID) to map binding sites of DNA- and chromatin-binding proteins. DamID identifies binding sites by expressing the proposed DNA-binding protein as a fusion protein with DNA methyltransferase.

FIG. 37—schematic of DamID mapping.

FIG. 38—PEG10-DAMID fusion constructs were analyzed for their ability to bind DNA and RNA by cross-referencing DamID mapping data with ATAC-seq data.

FIG. 39—results of mass-spectrometry analysis of enriched proteins in VLP fractions from N2A cells.

FIG. 40—schematic for how PEG10 mediates secretion from cells.

FIG. 41—schematic showing constructs that form RNA-containing gag vesicles.

FIG. 42—graph showing the ability of various gag-homology proteins to produce RNA-containing vesicles in the absence of VSV-G.

FIG. 43—graph showing the ability of various gag-homology proteins to produce RNA-containing vesicles in the presence of VSV-G.

FIG. 44—schematic showing protocol for genome-wide screen for native proteins that cross the blood-brain barrier.

FIG. 45—modification of protocol shown in FIG. 44 by transfecting passaged cells in step 1 with a 2^{nd} generation packaging vector to reactivate the provirus.

FIG. 46—shows the frequency with which guide RNAs end up internalized in target cells.

FIG. 47—shows a nuclear sort of CNS sub-populations 14 days post tail-vein.

FIG. 48—fluorescence micrographs showing the ability of different fusogens (Arghap32 and Clmp) to further efficiency of internalization.

FIG. 49—schematic showing protocol for transfection of constructs and evaluating ability of generating INDELs. Fusion of Cas9 to PEG10 and overexpression in cells allows for generation of INDELs in target cells.

FIG. 50—analysis of various gag-homology proteins for their ability to act as native fusogens.

FIG. 51—fluorescence micrographs showing the ability of different fusogens (Arghap32 and CXADR) to further efficiency of internalization.

FIG. 52—graph showing results of analysis of various gags carrying Cas9 for their ability to be secreted from cells.

FIG. 53—graph showing analysis of select gags from FIG. 52 for their ability to be secreted from cells in the presence of VSV-G.

FIG. 54—graphs showing percent INDEL generation from gags from FIG. 53 (left) when compared to HIV (right).

FIG. 55—analysis of the ability of various gag-IRES-Cas9 constructs to generate INDELs in the presence of various fusogens.

FIG. 56—schematic of PEG10 and Western blot showing cleavage pattern of overexpressed N- and C-terminal tagged mouse PEG10 in HEK293FT cells.

FIGS. 57A-57F—(57A) Western blot of PEG10 cleavage pattern and graph showing peptide abundance of full PEG10; (57B) Western blot of PEG10 cleavage pattern and graph showing peptide abundance of the first reading frame of PEG10; (57C) Western blot of PEG10 cleavage pattern and graph showing peptide abundance of NC cleavage products; (57D) Western blot of PEG10 cleavage pattern and graph showing peptide abundance after cleavage at the protease domain of the second reading frame of PEG10; (57E) Western blot of PEG10 cleavage pattern and graph showing peptide abundance after cleavage at the RT domain of the second reading frame of PEG10; (57F) Western blot of PEG10 cleavage pattern and graph showing peptide abundance after C-terminal cleavage of the second reading frame of PEG10.

FIGS. 58A-58B—Western blot and schematic of protease cleavage sites of PEG10 and the resulting protein fragments (58A) with and (58B) a putative cleave prior to the Gag domain.

FIG. 59—schematic of the PEG10 ORF1/2 gene and Western blots showing cleavage patterns of proteins isolated from VLP fraction and whole cell lysate.

FIG. 60—schematic of the PEG10 protein showing that a CCHC deletion in the NC domain renders it unable to bind a specific sequence (SEQ ID NO:2) bound by a known myelin expression factor (MYEF).

FIG. 61—protocol for binding experiments to determine whether PEG10 binds DNA and graph confirming that PEG10 binds DNA.

FIG. 62—schematic showing estimation of location of ORF1 cleavage site and experiment done to confirm the location.

FIG. 63—schematic showing location of ORF1 cleavage site and assessment of payload secretion.

FIG. 64—fluorescent micrographs showing expression of GFP fusion constructs of various ORFs.

FIG. 65—schematic of hypotheses for the putative functions of various domains when they interact with DNA.

FIG. 66—schematic of PEG10 with mutations in various domains to determine its function.

FIG. 67—schematic showing that if PEG10 is nuclear and can bind DNA, (like MYEF), then if follows that PEG10 regulates transcription.

FIG. 68—schematic showing that mutations in the nucleocapsid domain led to a reduced ability to bind the MYEF motif (SEQ ID NO:3).

FIG. 69—footprinting assay to determine function of individual motifs in the PEG10 protein.

FIG. 70—Western blot showing quantification of PEG10 in the blood of transgenic mice.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “high,” “higher,” “increased,” “elevated,” or “elevation” refer to increases above basal levels, e.g., as compared to a control. The terms “low,” “lower,” “reduced,” or “reduction refer to decreases below basal levels, e.g., as compared to a control.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control patient (can be a stored sample or previous sample measurement) with a known outcome; normal tissue, fluid, or cells isolated from a subject, such as a normal patient or the patient having a condition of interest.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide compositions, systems, and methods for delivering cargo to target cells. The present disclosure includes polynucleotides encoding one or more endogenous retroviral elements for forming a delivery vesicle and one or more capture moieties for packaging a cargo within the delivery vesicle. Such vesicles may be virus-like particles. The vesicles may be used for delivering therapeutic agents into target cells. The polynucleotide can comprise engineered genes that allow recruitment of cargo molecules or can be fused to cargo molecules that can be packaged in generated vesicles. Tailoring the polynucleotide compositions will allow for tailoring of cargo and delivery, including both cell-specific and cell-non-specific delivery methods. In specific embodiments, only one of the retroviral elements is an endogenous retroviral element. The endogenous retroviral element may be a retroviral gag protein or a retroviral envelope protein. The compositions, systems, and methods also comprise a retroviral reverse transcriptase. Preferably, the composition has reduced immunogenicity.

### Engineered Delivery Systems

In one aspect, embodiments disclosed herein relate to engineered polynucleotides and vectors encoding vesicle forming delivery systems derived from endogenous retroviral elements. In another aspect, embodiments disclosed herein are directed to use of such engineered polynucleotides in methods of loading and/or packaging desired cargo molecules. In another aspect, embodiment disclosed herein are directed to such cargo carrying delivery vesicles and methods of using said delivery vesicle to deliver cargo molecules to target cells.

### Engineered Polynucleotides

Embodiments disclosed here comprise engineered polynucleotides that encode one or more endogenous retroviral elements for forming a delivery vesicle and one or more capture moieties for packaging a cargo within the delivery vesicle. The engineered polynucleotide may further include regulatory elements such as promoters, enhancers, inernal ribosome entry sites (IRES), repressors, inducers, etc. to control expression of the vesicle forming system. The engineered polynucleotides are designed for delivery to a cell, acellular system, or any other suitable bioreactor to allow expression of the delivery system components and formation of said delivery vesicles including packaging of desired cargo molecules into said delivery vesicles.

In some embodiments, the one or more endogenous retroviral elements for forming a delivery vesicle comprises a retroviral envelope protein. In some embodiments, the one or more endogenous retroviral elements for forming a delivery vesicle comprises a retroviral gag protein. In some embodiments, the retroviral gag protein and the retroviral envelope protein are both endogenous. In some embodiments, the gag protein is endogenous and the envelope protein is of viral origin. In some embodiments, the envelope protein is endogenous and the gag protein is of viral origin. The system may further comprise cargo domain elements, such as peptide or nucleotide-based elements that specifically bind a cargo of interest and as described in further detail below.

The system may further include one or more targeting moieties, which is capable of specifically binding to a target cell. In some embodiments, the cargo may be linked to one or more envelope proteins by a linker. In some embodiments, the system may include regulatory molecules that control expression of the vesicle-forming system.

The term “regulatory element” is intended to include promoters, enhancers, internal ribosomal entry sites (IRES), other expression control elements (e.g., transcription termination signals, such as polyadenylation signals and poly-U sequences) and cellular localization signals (e.g. nuclear localization signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6, 7SK, and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF la promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). Specific configurations of the gRNAs, reporter gene and pol II and pol III promoters in the context of the present invention are described in greater detail elsewhere herein.

In some embodiments, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

In general, the system may include vesicle-generating polynucleotides, vesicle-generating plasmids, vesicles generated by such plasmids, or both. The sequences described below can be cloned into a vector. A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

The polynucleotide may be an RNA or DNA molecule. The polynucleotide can be a naturally occurring or recombinant polynucleotide. The polynucleotide can encode a protein or RNA molecule.

The polynucleotides may comprise encoding sequences for one or more components of a vesicle herein. In some examples, a polynucleotide comprises a sequence encoding a barcoding construct. The polynucleotide may further comprise a sequence encoding another element, such as a perturbation element. As used herein, a polynucleotide may be DNA, RNA, or a hybrid thereof, including without limitation, cDNA, mRNA, genomic DNA, mitochondrial DNA, sgRNA, siRNA, shRNA, miRNA, tRNA, rRNA, snRNA, lncRNA, and synthetic (such as chemically synthesized) DNA or RNA or hybrids thereof. The polynucleotides may include natural nucleotides (such as A, T/U, C, and G), modified nucleotides, analogs of natural nucleotides, such as labeled nucleotides, or any combination thereof.

The invention also provides delivery vesicles for delivery of the polynucleotides encoding the endogenous proteins. Such delivery vesicles or systems within the scope of the present invention may be provided in any form, including but not limited to solid, semi-solid, emulsion, or colloidal particles. As such, any of the delivery systems described herein, including but not limited to, e.g., lipid-based systems, liposomes, micelles, microvesicles, exosomes, or gene gun may be provided as particle delivery systems within the scope of the present invention.

In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. It will be appreciated that reference made herein to particles or nanoparticles can be interchangeable, where appropriate.

It will be understood that the size of the particle will differ depending as to whether it is measured before or after loading. Accordingly, in particular embodiments, the term “nanoparticles” may apply only to the particles pre-loading.

Nanoparticles encompassed in the present invention may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention.

Semi-solid and soft nanoparticles have been manufactured and are within the scope of the present invention. A prototype nanoparticle of semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants.

Self-assembling export compartments or nanoparticles with RNA may be constructed with polyethyleneimine (PEI) that is PEGylated with an Arg-Gly-Asp (RGD) peptide ligand attached at the distal end of the polyethylene glycol (PEG). This system has been used, for example, as a means to target tumor neovasculature expressing integrins and deliver siRNA inhibiting vascular endothelial growth factor receptor-2 (VEGF R2) expression and thereby achieve tumor angiogenesis (see, e.g., Schiffelers et al., Nucleic Acids Research, 2004, Vol. 32, No. 19). Nanoplexes may be prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. A dosage of about 100 to 200 mg of CRISPR Cas is envisioned for delivery in the self-assembling nanoparticles of Schiffelers et al.

The nanoplexes of Bartlett et al. (PNAS, Sep. 25, 2007, vol. 104, no. 39) may also be applied to the present invention. The nanoplexes of Bartlett et al. are prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. The DOTA-siRNA of Bartlett et al. was synthesized as follows: 1,4,7,10-tetraazacyclododecane-1,4,7,10-tetraacetic acid mono(N-hydroxysuccinimide ester) (DOTA-NHSester) was ordered from Macrocyclics (Dallas, Tex.). The amine modified RNA sense strand with a 100-fold molar excess of DOTA-NHS-ester in carbonate buffer (pH 9) was added to a microcentrifuge tube. The contents were reacted by stirring for 4 h at room temperature. The DOTA-RNAsense conjugate was ethanol-precipitated, resuspended in water, and annealed to the unmodified antisense strand to yield DOTA-siRNA. All liquids were pretreated with Chelex-100 (Bio-Rad, Hercules, Calif.) to remove trace metal contaminants. Tf-targeted and nontargeted siRNA nanoparticles may be formed by using cyclodextrin-containing polycations. Typically, nanoparticles were formed in water at a charge ratio of 3 (+/−) and an siRNA concentration of 0.5 g/liter. One percent of the adamantane-PEG molecules on the surface of the targeted nanoparticles were modified with Tf (adamantane-PEG-Tf). The nanoparticles were suspended in a 5% (wt/vol) glucose carrier solution for injection.

The lipid particles developed by Qiaobing Xu's lab at Tufts University may be used/adapted to the present delivery system. See Wang et al., J. Control Release, 2017 Jan. 31. pii: 50168-3659(17)30038-X. doi: 10.1016/j.jconre1.2017.01.037. [Epub ahead of print]; Altιno\check{g}lu et al., Biomater Sci., 4(12):1773-80, Nov. 15, 2016; Wang et al., PNAS, 113(11):2868-73 March 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014.

US Patent Publication No. 20110293703 also provides libraries of aminoalcohol lipidoid compounds prepared by the inventive methods. These aminoalcohol lipidoid compounds may be prepared and/or screened using high-throughput techniques involving liquid handlers, robots, microtiter plates, computers, etc. In certain embodiments, the aminoalcohol lipidoid compounds are screened for their ability to transfect polynucleotides or other agents (e.g., proteins, peptides, small molecules) into the cell.

US Patent Publication No. 2013/0302401 relates to a class of poly(beta-amino alcohols) (PBAAs) that are prepared using combinatorial polymerization. The inventive PBAAs may be used in biotechnology and biomedical applications as coatings (such as coatings of films or multilayer films for medical devices or implants), additives, materials, excipients, non-biofouling agents, micropatterning agents, and cellular encapsulation agents. When used as surface coatings, these PBAAs elicited different levels of inflammation, both in vitro and in vivo, depending on their chemical structures. The large chemical diversity of this class of materials allowed identification of polymer coatings that inhibit macrophage activation in vitro. Furthermore, these coatings reduce the recruitment of inflammatory cells, and reduce fibrosis, following the subcutaneous implantation of carboxylated polystyrene microparticles. These polymers may be used to form polyelectrolyte complex capsules for cell encapsulation. The invention may also have many other biological applications such as antimicrobial coatings, DNA or siRNA delivery, and stem cell tissue engineering. The teachings of US Patent Publication No. 20130302401 may be applied to a CRISPR Cas system or any other system of the present invention.

In another embodiment, lipid nanoparticles (LNPs) are contemplated. An antitransthyretin small interfering RNA has been encapsulated in lipid nanoparticles and delivered to humans (see, e.g., Coelho et al., N Engl J Med 2013; 369:819-29), and such a system may be adapted and applied to a CRISPR Cas system or any other system of the present invention. Doses of about 0.01 to about 1 mg per kg of body weight administered intravenously are contemplated. Medications to reduce the risk of infusion-related reactions are contemplated, such as dexamethasone, acetaminophen, diphenhydramine or cetirizine, and ranitidine are contemplated. Multiple doses of about 0.3 mg per kilogram every 4 weeks for five doses are also contemplated.

Zhu et al. (US20140348900) provides for a process for preparing liposomes, lipid discs, and other lipid nanoparticles using a multi-port manifold, wherein the lipid solution stream, containing an organic solvent, is mixed with two or more streams of aqueous solution (e.g., buffer). In some aspects, at least some of the streams of the lipid and aqueous solutions are not directly opposite of each other. Thus, the process does not require dilution of the organic solvent as an additional step. In some embodiments, one of the solutions may also contain an active pharmaceutical ingredient (API). This invention provides a robust process of liposome manufacturing with different lipid formulations and different payloads. Particle size, morphology, and the manufacturing scale can be controlled by altering the port size and number of the manifold ports, and by selecting the flow rate or flow velocity of the lipid and aqueous solutions.

LNPs have been shown to be highly effective in delivering siRNAs to the liver (see, e.g., Tabernero et al., Cancer Discovery, April 2013, Vol. 3, No. 4, pages 363-470) and are therefore contemplated for delivering RNA encoding CRISPR Cas to the liver. A dosage of about four doses of 6 mg/kg of the LNP every two weeks may be contemplated. Tabernero et al. demonstrated that tumor regression was observed after the first 2 cycles of LNPs dosed at 0.7 mg/kg, and by the end of 6 cycles the patient had achieved a partial response with complete regression of the lymph node metastasis and substantial shrinkage of the liver tumors. A complete response was obtained after 40 doses in this patient, who has remained in remission and completed treatment after receiving doses over 26 months. Two patients with RCC and extrahepatic sites of disease including kidney, lung, and lymph nodes that were progressing following prior therapy with VEGF pathway inhibitors had stable disease at all sites for approximately 8 to 12 months, and a patient with PNET and liver metastases continued on the extension study for 18 months (36 doses) with stable disease.

In some embodiments, the LNP contains a nucleic acid, wherein the charge ratio of nucleic acid backbone phosphates to cationic lipid nitrogen atoms is about 1:1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weigh between about 500 to 10,000 Da or between about 2000 to 5000 Da. In some embodiments, the shielding compound is PEG2000 or PEG5000.

In some embodiments, sugar-based particles may be used, for example GalNAc, as described herein and with reference to WO2014118272 (incorporated herein by reference) and Nair, J K et al., 2014, Journal of the American Chemical Society 136 (49), 16958-16961) and the teaching herein, especially in respect of delivery applies to all particles unless otherwise apparent. This may be considered to be a sugar-based particle and further details on other particle delivery systems and/or formulations are provided herein. GalNAc can therefore be considered to be a particle in the sense of the other particles described herein, such that general uses and other considerations, for instance delivery of said particles, apply to GalNAc particles as well. A solution-phase conjugation strategy may for example be used to attach triantennary GalNAc clusters (mol. wt. ˜2000) activated as PFP (pentafluorophenyl) esters onto 5′-hexylamino modified oligonucleotides (5′-HA ASOs, mol. wt. ˜8000 Da; Ostergaard et al., Bioconjugate Chem., 2015, 26 (8), pp 1451-1455). Similarly, poly(acrylate) polymers have been described for in vivo nucleic acid delivery (see WO2013158141 incorporated herein by reference). In further alternative embodiments, pre-mixing CRISPR nanoparticles (or protein complexes) with naturally occurring serum proteins may be used in order to improve delivery (Akinc A et al, 2010, Molecular Therapy vol. 18 no. 7, 1357-1364).

Literature that may be employed in conjunction with herein teachings include: Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., Small, 10:186-192.

Measurement of cell-to-cell transfer may be evaluated at multiple steps as described in Patsuzyn et al. (Cell 172(1-2):275-288; 2018). In specific embodiments, indirect testing of capsid formation in transfected HEK293 cells may done by chemical cross-linking followed by SDS-PAGE to probe for appearance of higher molecular weight bands corresponding to protein oligomers. Export in extracellular vesicles may be performed by purifying the extracellular vesicle fraction from the media following transfection and using western blots to look for the protein in addition to reported extracellular vesicle markers. Finally, the ability of capsid-containing extracellular vesicles to be taken up by recipient cells may be tested by placing either the media or the purified extracellular vesicle fraction from cells transfected with a GFP-tagged Gag onto untransfected cells and looking for uptake of fluorescence using microscopy and/or FACS. In addition to extracellular vesicle-mediated transfer, recombinant Arc can form capsids in vitro that transfer enclosed RNA to recipient cells in the absence of an endosomal membrane. The proteins may also be either purified from bacteria or translated in vitro and tested for this activity. The formation of capsid structures in the different assays may be confirmed using methods including, but not necessarily limited to, electron microscopy, dynamic light scattering, or Spectradyne particle analysis.

In specific embodiments, unassembled recombinant GAG-like proteins, nucleic acids and/or proteins are combined in solution in low salt conditions.

U.S. Pat. No. 8,709,843, incorporated herein by reference, provides a drug delivery system for targeted delivery of therapeutic agent-containing particles to tissues, cells, and intracellular compartments. The invention provides targeted particles comprising polymer conjugated to a surfactant, hydrophilic polymer or lipid. The teachings of U.S. Pat. No. 8,709,843 may be applied and/or adapted to incorporate and/or deliver one or more of the engineered delivery system molecules of the present invention described herein.

U.S. Pat. No. 5,543,158, incorporated herein by reference, provides biodegradable injectable particles having a biodegradable solid core containing a biologically active material and poly(alkylene glycol) moieties on the surface. The teachings of U.S. Pat. No. 5,543,158 may be applied and/or adapted to incorporate and/or deliver one or more of the engineered delivery system molecules of the present invention described herein.

International Patent Publication No. WO2012135025 (also published as US20120251560), incorporated herein by reference, describes conjugated polyethyleneimine (PEI) polymers and conjugated aza-macrocycles (collectively referred to as “conjugated lipomer” or “lipomers”). In certain embodiments, it can be envisioned that such conjugated lipomers can be used in the context of the engineered delivery system described herein to achieve in vitro, ex vivo and in vivo expression of one or more components of the engineered delivery system described herein and in some embodiments may result in production of engineered delivery particles from the engineered cell(s).

Further, the engineered delivery system molecule(s) described herein may be delivered using nanoclews, for example as described in Sun W et al, Cocoon-like self-degradable DNA nanoclew for anticancer drug delivery, J Am Chem Soc. 2014 Oct. 22; 136(42):14722-5. doi: 10.1021/ja5088024. Epub 2014 Oct. 13; or in Sun W et al, Self-Assembled DNA Nanoclews for the Efficient Delivery of CRISPR-Cas9 for Genome Editing, Angew Chem Int Ed Engl. 2015 Oct. 5; 54(41):12029-33. doi: 10.1002/anie.201506030. Epub 2015 Aug. 27. The teachings of Sun et al. can be applied and/or adapted to generate and/or deliver the CRISRP-Cas system molecules described herein.

One or more of the engineered delivery system molecules described herein can be contained or otherwise incorporated in exosomes for delivery. Exosomes containing one or more engineered delivery molecules described herein can be used to deliver the one or more engineered delivery system molecule(s) to a cell and/or subject.

Exosomes are endogenous nano-vesicles that transport RNAs and proteins, and which can deliver RNA to the brain and other target organs. To reduce immunogenicity, Alvarez-Erviti et al. (2011, Nat Biotechnol 29: 341) used self-derived dendritic cells for exosome production. Targeting to the brain was achieved by engineering the dendritic cells to express Lamp2b, an exosomal membrane protein, fused to the neuron-specific RVG peptide. Purified exosomes were loaded with exogenous RNA by electroporation. Intravenously injected RVG-targeted exosomes delivered GAPDH siRNA specifically to neurons, microglia, oligodendrocytes in the brain, resulting in a specific gene knockdown. Pre-exposure to RVG exosomes did not attenuate knockdown, and non-specific uptake in other tissues was not observed. The therapeutic potential of exosome-mediated siRNA delivery was demonstrated by the strong mRNA (60%) and protein (62%) knockdown of BACE1, a therapeutic target in Alzheimer's disease. The teachings of Alvarez-Erviti et al. can be applied and/or adapted to generate and/or deliver the CRISPR-Cas system molecules described herein.

In some embodiments, the delivery system elicits a poor immune response or has reduced immunogenicity.

In some embodiments, the delivery vesicle is a virus-like particle (VLP). As used herein, the term “virus-like particle” (VLP) refers to a structure that in at least one attribute resembles a virus, but which has not been demonstrated to be infectious. A VLP may be a nonreplicating, noninfectious viral shell that contains a viral capsid but lacks all or part of the viral genome, in particular, the replicative components of the viral genome. VLPs are generally composed of one or more viral proteins, such as, but not limited to those proteins referred to as capsid, coat, shell, surface, and structural proteins (e.g., VP1, VP2). A VLP may also resemble the structure of a bacteriophage, being non-replicative and noninfectious, and lacking at least the gene or genes coding for the replication machinery of the bacteriophage, and also lacking the gene or genes encoding the protein or proteins responsible for viral attachment to or entry into the host.

Envelopes from various retrovirus sources can be used for pseudotyping a vector. The exact rules for pseudotyping (i.e., which envelope proteins will interact with the nascent vector particle at the cytoplasmic side of the cell membrane to give a viable viral particle (Tato, Virology 88:71, 1978) and which will not (Vana, Nature 336:36, 1988), are not well characterized. However, since a piece of cell membrane buds off to form the viral envelope, molecules normally in the membrane are carried along on the viral envelope. Thus, a number of different potential ligands can be put on the surface of viral vectors by manipulating the cell line making gag and pol in which the vectors are produced, or choosing various types of cell lines with particular surface markers. One type of surface marker that can be expressed in helper cells and that can give a useful vector-cell interaction is the receptor for another potentially pathogenic virus. The pathogenic virus displays on the infected cell surface its virally specific protein (e.g., env) that normally interacts with the cell surface marker or receptor to give viral infection. This reverses the specificity of the infection of the vector with respect to the potentially pathogenic virus by using the same viral protein-receptor interaction, but with the receptors on the vector and the viral protein on the cell.

One virus known to participate in pseudotype formation is vesicular stomatitis virus (VSV), the prototypic member of the rhabdovirus family. It is an enveloped virus with a negative stranded RNA genome that causes a self-limiting disease in live-stock and is essentially non-pathogenic in humans. Balachandran and Barber (2000, IUBMB Life 50: 135-8). Rhabdoviruses have single, negative-strand RNA genomes of 11,000 to 12,000 nucleotides (Rose and Schubert, 1987, Rhabdovirus genomes and their products, in The Viruses: The Rhabdoviruses, Plenum Publishing Corp., NY, pp. 129-166). The virus particles contain a helical, nucleocapsid core composed of the genomic RNA and protein. Generally, three proteins, termed N (nucleocapsid, which encases the genome tightly), P (formerly termed NS, originally indicating nonstructural), and L (large) are found to be associated with the nucleocapsid. An additional matrix (M) protein lies within the membrane envelope, perhaps interacting both with the membrane and the nucleocapsid core. A single glycoprotein (G) species spans the membrane and forms the spikes on the surface of the virus particle.

### Endogenous Retroviral Elements

Human endogenous retrovirus (HERV) sequences make up 8.29% of the draft human genome. Their prevalence has resulted from the accumulation of past retroviral infectious agents that have entered the germline, established a truce with the host cell, and are expressed from that host genome. HERVs can be grouped according to sequence homologies in approximately 100 different families, each containing a few to several hundred elements. Genes co-opted by the host from endogenous retroviruses are found to be active participants in some cellular processes including viral defense by Fv1 and Fv4 in the mouse, and cellular fusion in human placental development mediated through syncitin. Although HERV transcripts have been detected in both normal and cancerous tissues, including T cells, their role in normal cell function and carcinogenesis is unclear. While the cellular conditions that promote HERV transcription are not well understood, the APOBECs have been shown to play a role in the control of endogenous retroviruses.

Strong similarities between current HERV and retroviruses can be deduced from phylogenetic analyzes in the reverse transcriptase domain of the pol gene or the transmembrane (TM) moiety of the env gene, which disclose the interleaving of both kinds of elements and suggest a common history and Shared ancestors (Tristem, M. (2000) J. Virol. 74, 3715-3730; Benit et al. (2001) J. Virol. 75 (11709-11719). Similarities are also observed at the functional level.

As a result of the close relationship between HERV and infectious retroviruses, and despite the fact that most HERVs have accumulated mutations, deletions and/or truncations, it is still possible that some elements still have infectious retrovirus functions, which the host may have diverted to their own benefit.

Genes encoding viral polypeptides capable of self-assembly into defective, non-self-propagating viral particles can be obtained from the genomic DNA of a DNA virus or the genomic cDNA of an RNA virus or from available subgenomic clones containing the genes. These genes will include those encoding viral capsid proteins (i.e., proteins that comprise the viral protein shell) and, in the case of enveloped viruses, such as retroviruses, the genes encoding viral envelope glycoproteins. Additional viral genes may also be required for capsid protein maturation and particle self-assembly. These may encode viral proteases responsible for processing of capsid protein or envelope glycoproteins. As an example, the genomic structure of picornaviruses has been well characterized, and the patterns of protein synthesis leading to virion assembly are clear. Rueckert, R. in Virology (1985), B. N. Fields et al. (eds.) Raven Press, New York, pp 705-738. In picornaviruses, the viral capsid proteins are encoded by an RNA genome containing a single long reading frame, and are synthesized as part of a polyprotein which is processed to yield the mature capsid proteins by a combination of cellular and viral proteases. Thus, the picornavirus genes required for capsid self-assembly include both the capsid structural genes and the viral proteases required for their maturation. Another virus class from which genes encoding self-assembling capsid proteins can be isolated is the lentiviruses, of which HIV is an example. Like the picornaviral capsid proteins, the HIV gag protein is synthesized as a precursor polypeptide that is subsequently processed, by a viral protease, into the mature capsid polypeptides. However, the gag precursor polypeptide can self-assemble into virus-like particles in the absence of protein processing. Gheysen et al., Cell 59:103 (1989); Delchambre et al., The EMBO J. 8:2653-2660 (1989). Unlike picornavirus capsids, HIV capsids are surrounded by a loose membranous envelope that contains the viral glycoproteins. These are encoded by the viral env gene.

In alternative embodiments, additional human proteins with Gag homology may be used to assemble viral-like capsids that mediate intercellular transfer of cargo. Such proteins include, but are not necessarily limited to, the extended PNMA gene family including ZCC18, ZCH12, PNM8B, PNM8B, PNM6A, PMA6F, PMA6E, PNMA2, PNM8A, PNMA3, PNMA5, PNMA1, MOAP1, and CCDC8. In specific embodiments, the GAG-like protein is Arc.

In some embodiments, the endogenous retroviral element is an endogenous retroviral gag protein. In some embodiments, the endogenous retroviral element is an endogenous retroviral envelope protein. In some embodiments, the endogenous retroviral element is a retroviral reverse transcriptase. In some embodiments, one or more retroviral elements may be endogenous. In some embodiments, two or more retroviral elements may be endogenous.

In some embodiments, one or more endogenous retroviral elements for forming a delivery vesicle may comprise two or more of a retroviral gag protein, a retroviral envelope protein, a retroviral reverse transcriptase or a combination thereof.

### Retroviral Gag Protein

Group-specific antigen (gag) proteins are the core structural proteins or the major components of the retroviral capsid. The HIV p17 matrix protein (MA) is a 17 kDa protein, of 132 amino acids, which comprises the N-terminus of the Gag polyprotein. It is responsible for targeting Gag polyprotein to the plasma membrane but also makes contacts with the HIV trans-membrane glycoprotein gp41 in the assembled virus and may play a critical role in recruiting Env glycoproteins to viral budding sites.

Several studies have shown that expression of the gag gene alone in a number of systems results in the efficient assembly and release of membrane enveloped virions (Craven, R. C., et al. (1996). Dynamic interactions of the Gag polyprotein. Current Topics in Microbiology and Immunology 214, pp. 65-94; Delchambre, M., et al. (1989). The Gag precursors of simian immunodeficiency virus assemble into virus-like particles. EMBO 8, pp. 2653-60; Dickson, C., et al. (1984). “Protein biosynthesis and assembly,” RNA tumor viruses (R. Weiss, N. Teich, H. Varmus, and J. Coffin, Eds.), Vol. 1, pp. 513-648. 2 vols. Cold Spring Harbor Laboratory, Cold Spring Harbor, N.Y.; Gheysen, H. P., et al. (1989), “Assembly and release of HIV-1 precursor Pr55gag virus-like particles from recombinant baculovirus-infected insect cells,” Cell 59, pp. 103-12; Haffar, O., et al. (1990), “Human immunodeficiency virus-like, non-replication, Gag-Env particles assemble in a recombinant vaccinia virus expression system,” J. Virol. 64, pp. 2653-59; Hunter, E. (1994), “Macromolecular interactions in the assembly of HIV and other retroviruses,” Sem. in Virology 5, pp. 71-83; Krausslich, H.-G., et al. (1996), “Intracellular transport of retroviral capsid components,” Current Topics in Microbiology and Immunology 214, pp. 25-64; Madisen, L., et al. (1987), “Expression of the human immunodeficiency virus gag gene in insect cells,” Virology 158, pp. 248-250; Smith, A. J., et al. (1990), “Human immunodeficiency virus type 1 Pr55gag and Pr160gag-pol expressed from a simian virus 40 late-replacement vector are efficiently processed and assembled into virus-like particles,” J. Virol. 64, pp. 2743-50; Sommerfelt, M. A., et al. (1992), “Importance of the p12 protein in Mason-Pfizer monkey virus assembly and infectivity,” J. Virol. 66, pp. 7005-11; Wills, J. W., et al. (1989), “Creation and expression of myristylated forms of Rous sarcoma virus Gag protein in mammalian cells,” J. Virol. 63, pp. 4331-43). Thus, the product of this gene has the necessary structural information to mediate intracellular transport, to direct assembly into the capsid shell, and to catalyze the process of membrane extrusion known as budding.

Once Gag is translated, Gag polyproteins are myristoylated at their N-terminal glycine residues by N-myristoyltransferase 1, a modification that is critical for plasma membrane targeting. In the membrane-unbound form, the MA myristoyl fatty acid tail is sequestered in a hydrophobic pocket in the core of the MA protein. Recognition of plasma membrane proteins by MA activates a “myristoyl switch”, wherein the myristoyl group is extruded from its hydrophobic pocket in MA and embedded in the plasma membrane.

The HIV nucleocapsid protein (NC) is a 7 kDa zinc finger protein in the Gag polyprotein and which, after viral maturation, forms the viral nucleocapsid. NC recruits full-length viral genomic RNA to nascent virions.

The neuronal gene Arc bears homology to the Gag component of Ty3/gypsy retrotransposons and exhibits biochemical properties that are reminiscent of retroviral Gag proteins. The Arc protein assembles into virus-like capsids both in cells and when recombinantly expressed in bacteria. Arc capsids are able to encapsulate their own mRNA, mediating their intercellular transfer in extracellular vesicles. Purified Arc proteins may be used to reconstitute capsids with different DNA or RNA or proteins or some mixture thereof and can be packaged into the capsid for delivery into cells. In some embodiments, capsids may be assembled using lipids to aid uptake by cells. Various embodiments may utilize different Arc orthologs.

In some embodiments, the polynucleotides described herein may comprise a Gag-homology protein or functional domain thereof. The term “functional domain” refers to a polypeptide sequence that has an activity other than binding to the nucleic acid sequence recognized by the nucleic acid-binding domain. By combining a nucleic acid-binding domain with one or more effector domains, the polypeptides of the invention may be used to target the one or more functions or activities mediated by the effector domain to a particular target DNA sequence to which the nucleic acid-binding domain specifically binds.

Molecular and genetic determinants of Gag-mediated intercellular communication may be determined by characterizing the mechanisms of capsid-mediated intercellular mRNA transfer, with particular focus on features that could enable use of this system for programmable delivery of cargo. Different Gag proteins evolved diverse RNA-binding domains for mediating specific encapsidation of their RNA genomes. The RNA-binding sequence specificity of the human Gag homology proteins can be tested through protein pull-down and sequencing of associated RNA and/or through sequencing of the extracellular vesicle fraction from HEK293 cells that over-express each protein. The nucleic-acid-binding domains can be swapped between proteins, or additional RNA-binding domains with known specificity can be fused to test the extent to which binding specificity can be reprogrammed. Accordingly, the Gag-homology protein or functional domain thereof can comprise both the export compartment domain and nucleic acid-binding complain.

The Gag-homology protein can be selected from Arc, ASPRV1, a Sushi-Class protein, a SCAN protein, or a PNMA protein. In particular instances, the Gag-homology protein is a PNMA protein, for example, ZCC18, ZCH12, PNM8B, PNM6A, PNMA6E_i2, PMA6F, PMAGE, PNMA1, PNMA2, PNM8A, PNMA3, PNMA4, PNMA5, PNMA6, PNMA7, PNMA1, MOAP1, or CCD8. In embodiments, the Gag-homology protein is an Arc protein, in certain embodiments, hARC or dARC1. The Gag-homology protein can comprise ASPRV1. In other instances, the Gag-homology protein is PEG10, RTL3, RTL10, or RTL1. In certain embodiments, the Gag Homology protein is a SCAN protein, for example, PGBD1. In instances, the PEG10 Gag homology protein is PEG10_i6 or PEG10_i2.

In some embodiments, the Gag-homology protein or functional domain thereof may comprise both the export compartment domain and the nucleic acid-binding domain. In specific embodiments, the nucleic acid binding-domain may be modified relative to the native nucleic acid-binding domain of the Gag-homology protein. In specific embodiments, the nucleic acid-binding domain may be a non-native nucleic acid-binding domain relative to the Gag-homology protein. In some embodiments, the Gag-homology protein may be Arc or a paraneoplastic Ma antigen (PNMA) protein.

In some embodiments, the recombinant GAG-like proteins may be expressed and purified from bacteria, yeast, insect cells, or mammalian cells. The recombinant GAG-like proteins may be purified under denaturing conditions and transferred to non-denaturing conditions by buffer exchange.

In some embodiments, the retroviral gag protein is endogenous.

In some embodiments, the retroviral gag protein may contain the NC and MA domains.

In some embodiments, the retroviral gag protein may be a gag-homology protein, as described herein.

In some embodiments, the gag-homology protein may include, but is not necessarily limited to, Arc1, Asprv1, PNMA1, PNMA3, PNMA4, PNMA5, PNMA6, PNMA7, PEG10, RTL1, MOAP1, or ZCCHC12. In specific embodiments, the gag-homology protein is Arc1, PNMA6a, or PNMA3. In specific embodiments, the gag-homology protein is PEG10.

In some embodiments, the gag-homology protein may contain a DNA-binding motif. As a specific example, and as discussed in Example 4, PEG10 comprises a DNA binding motif that allows for packaging of DNA of specific sequences.

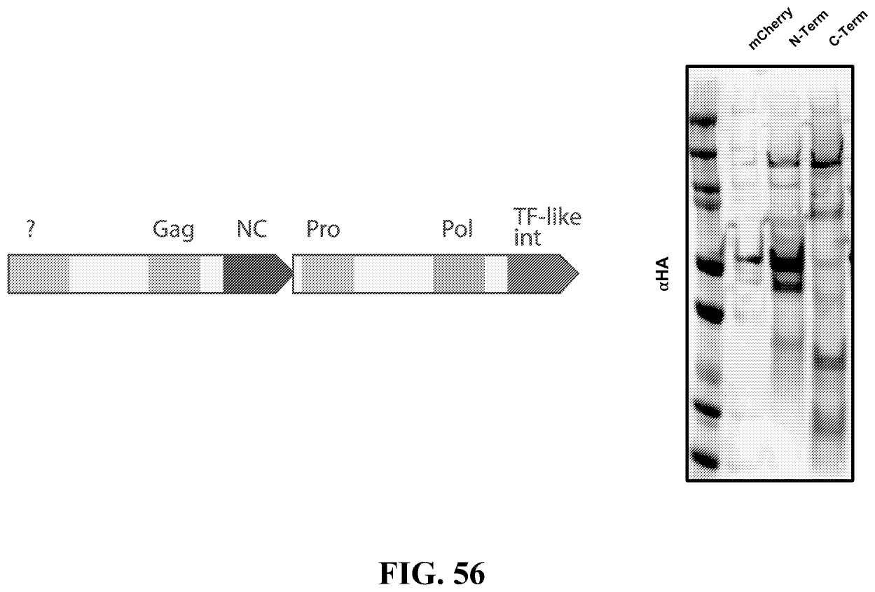

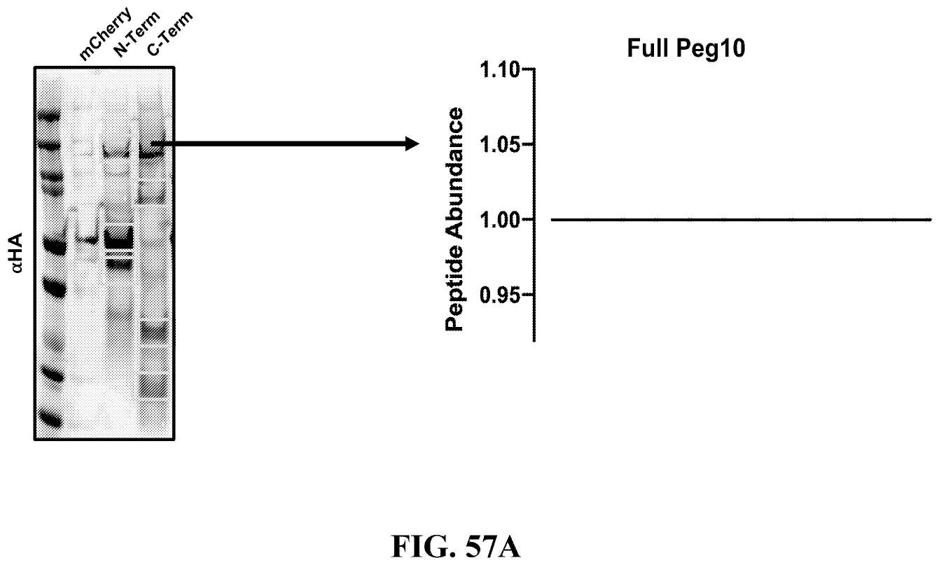

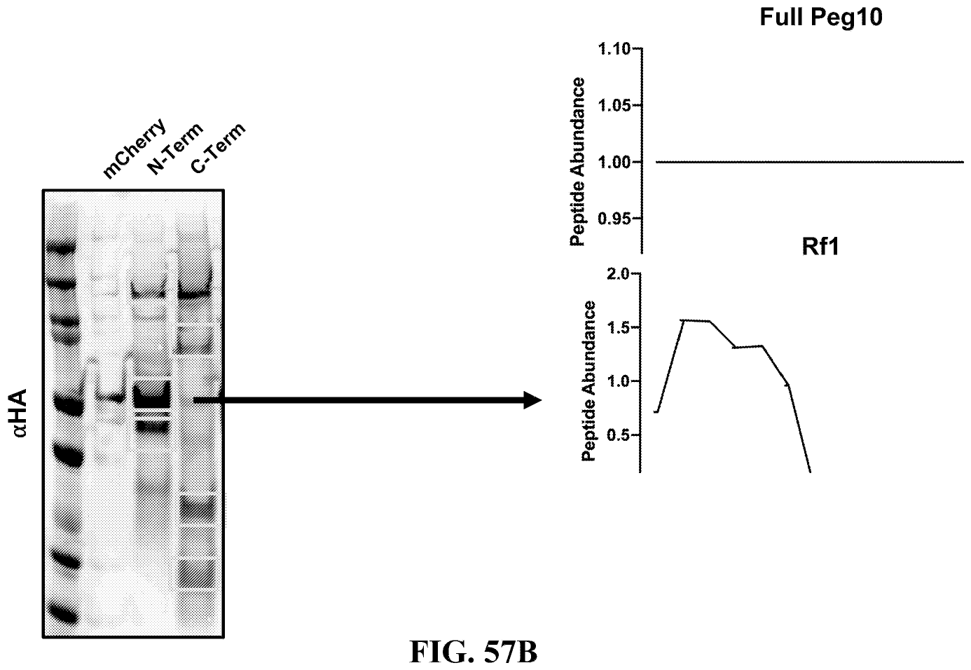

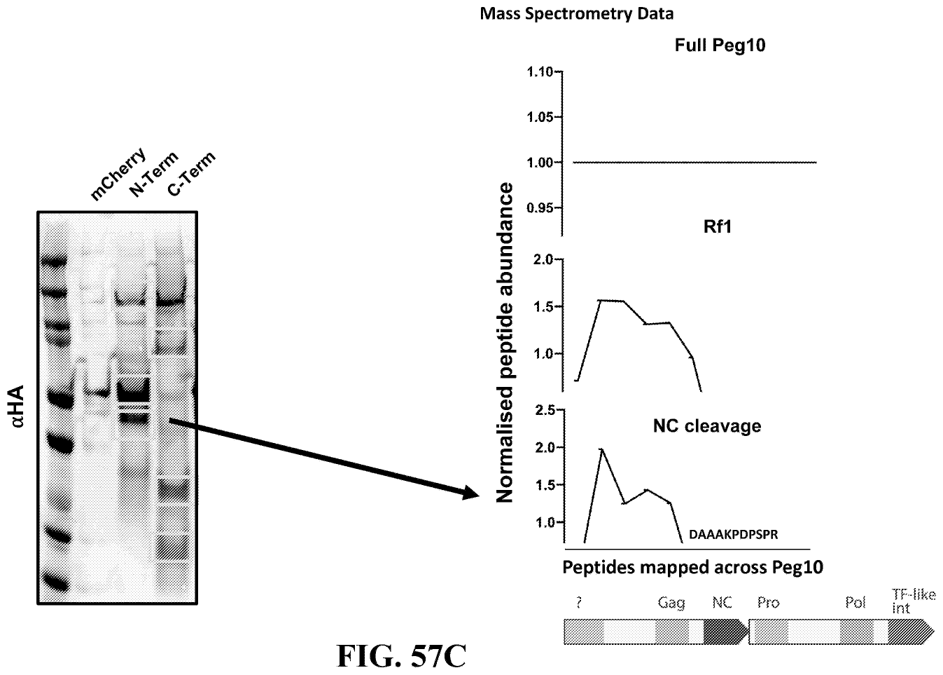

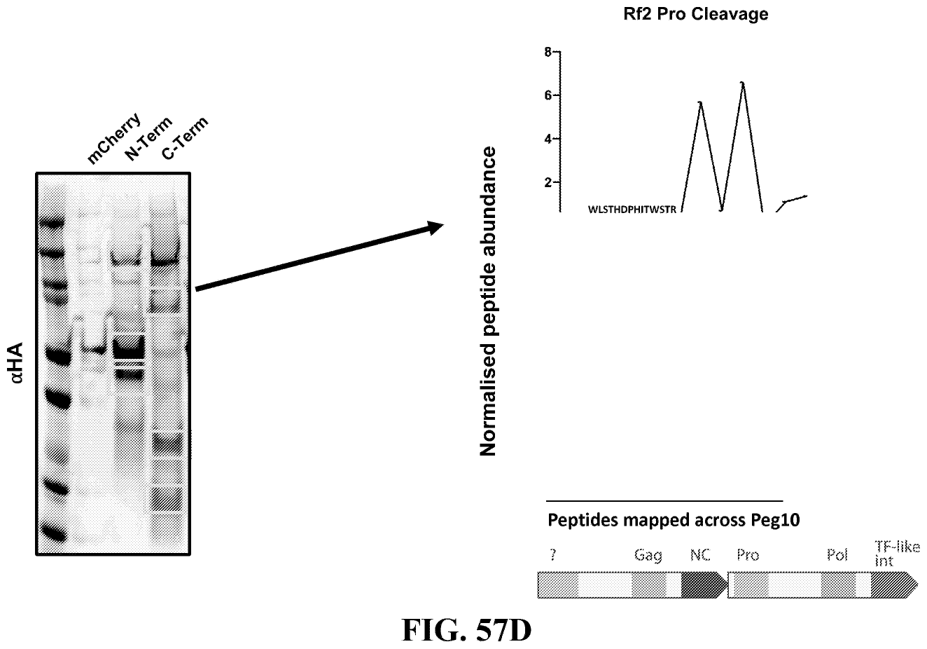

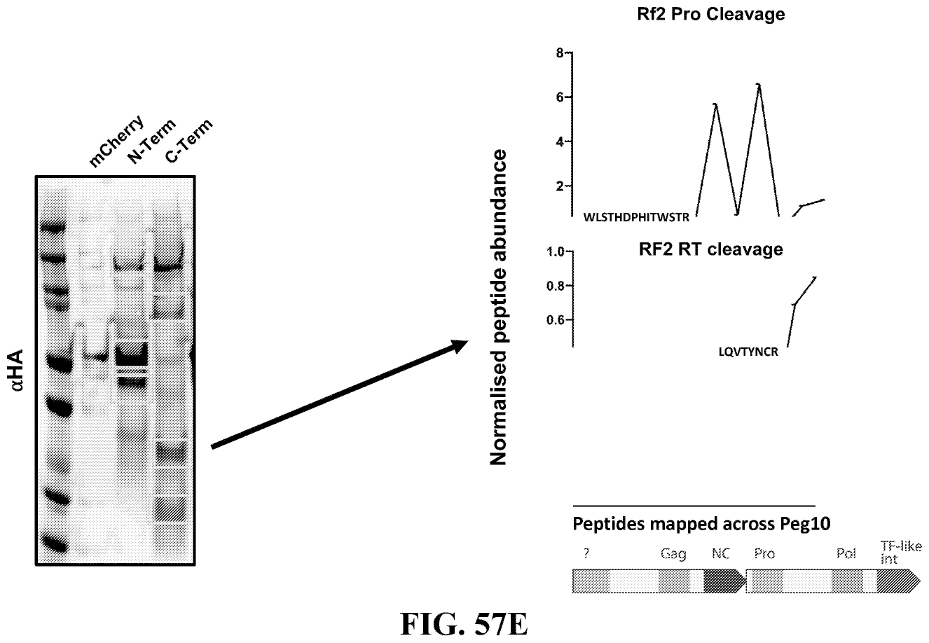

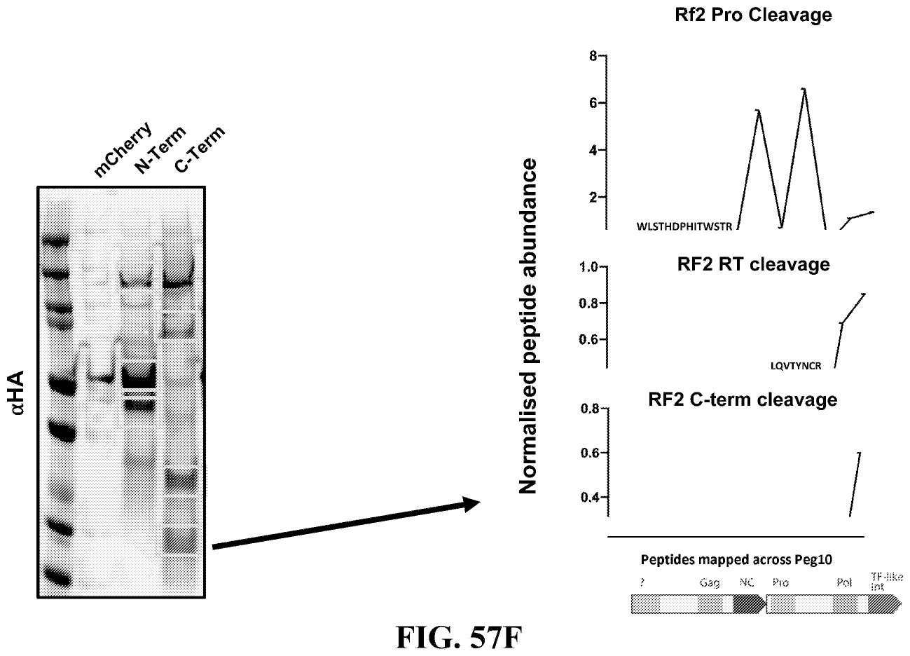

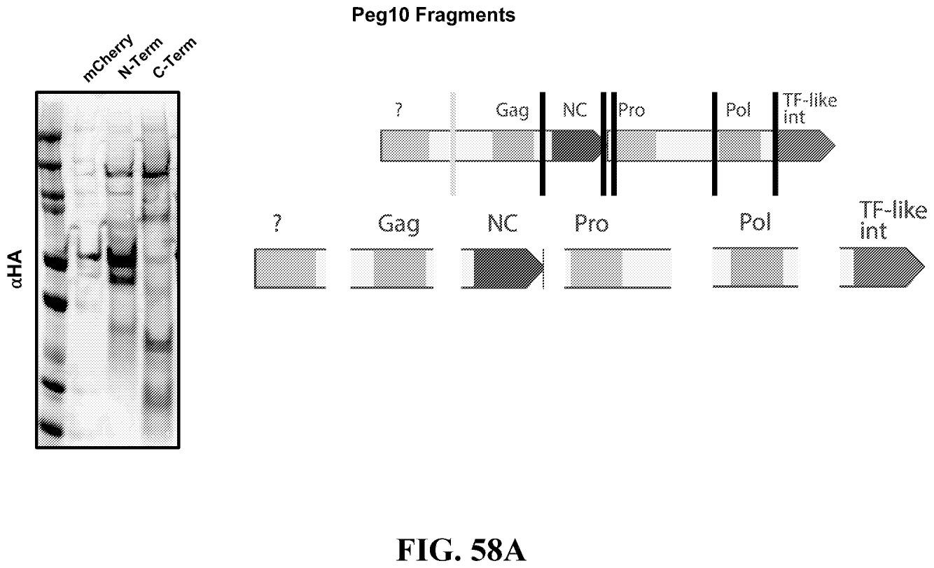

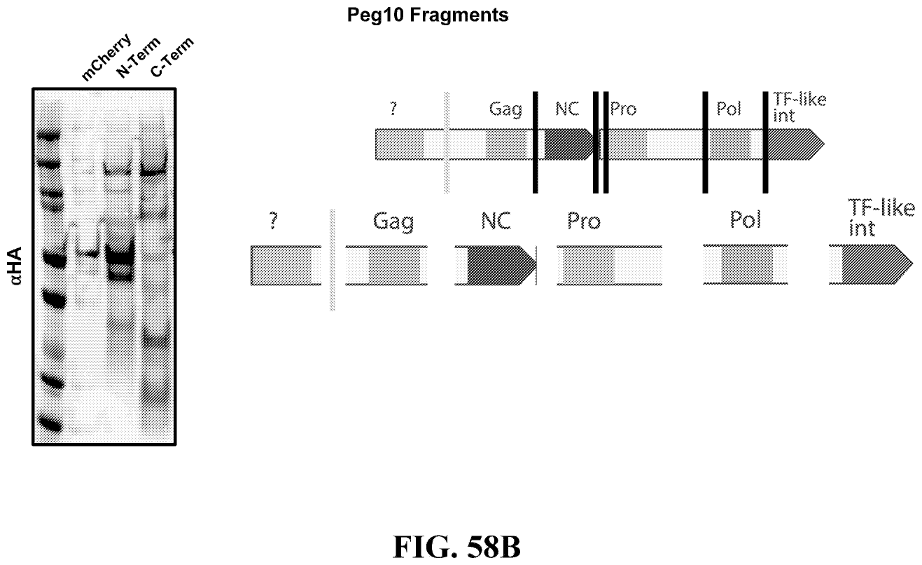

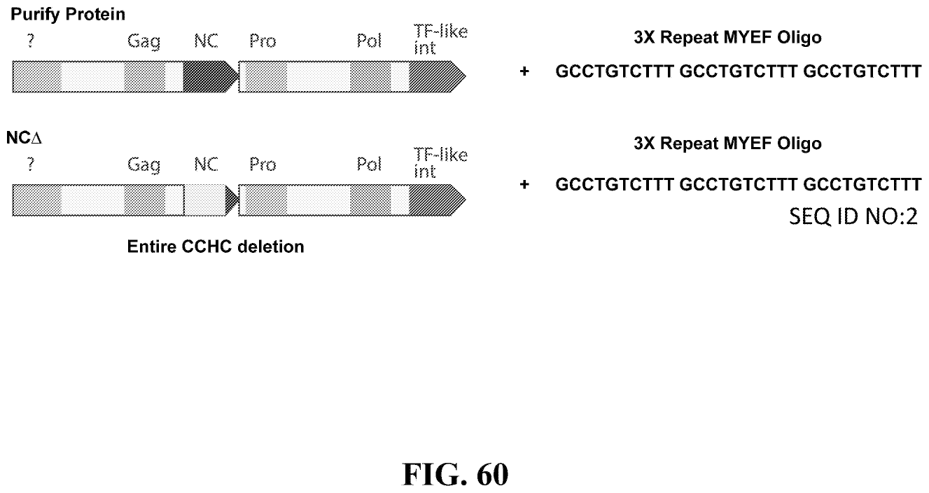

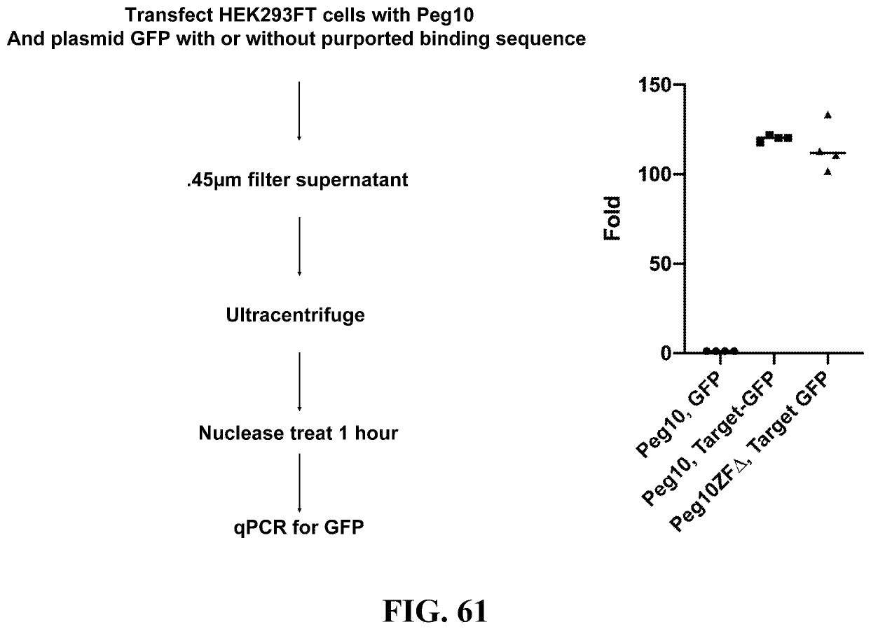

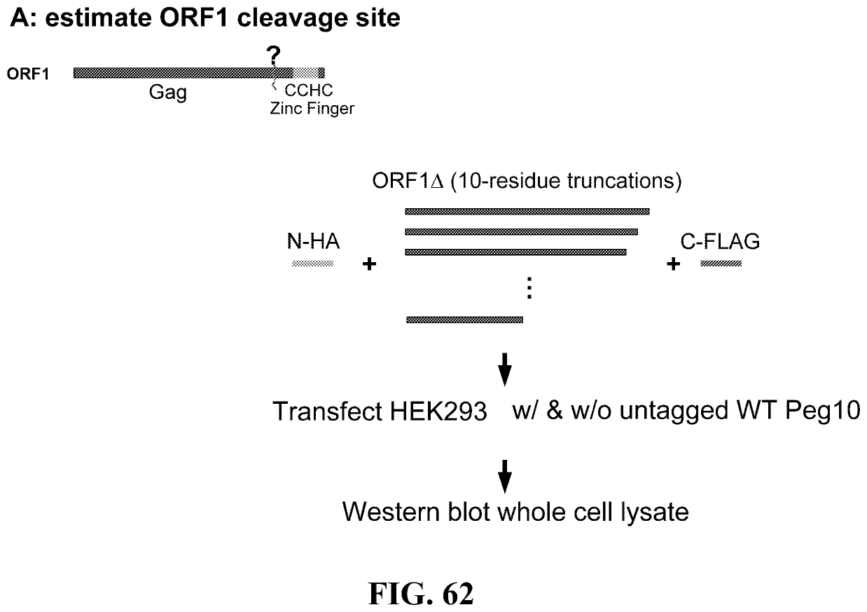

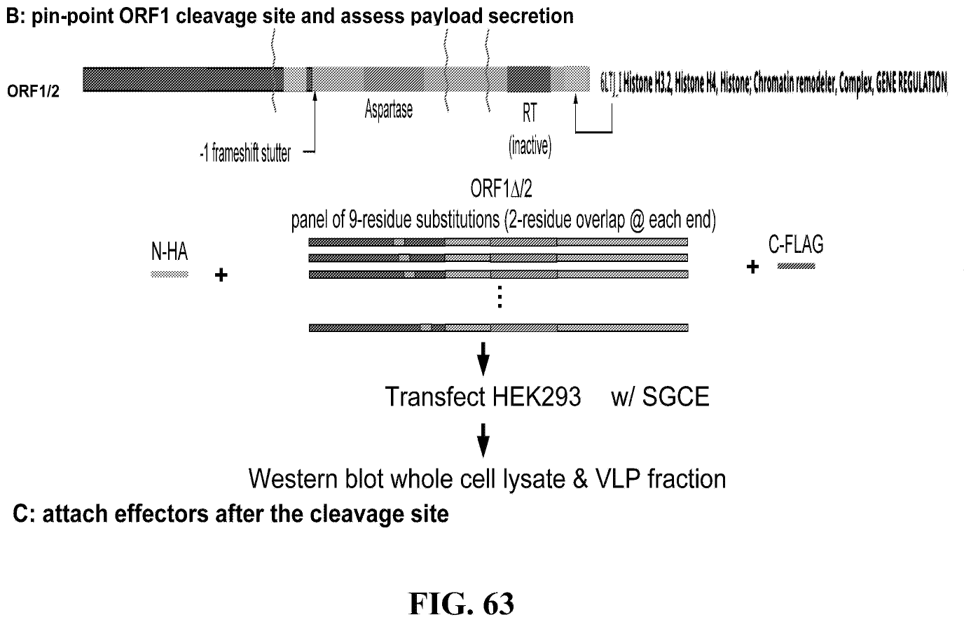

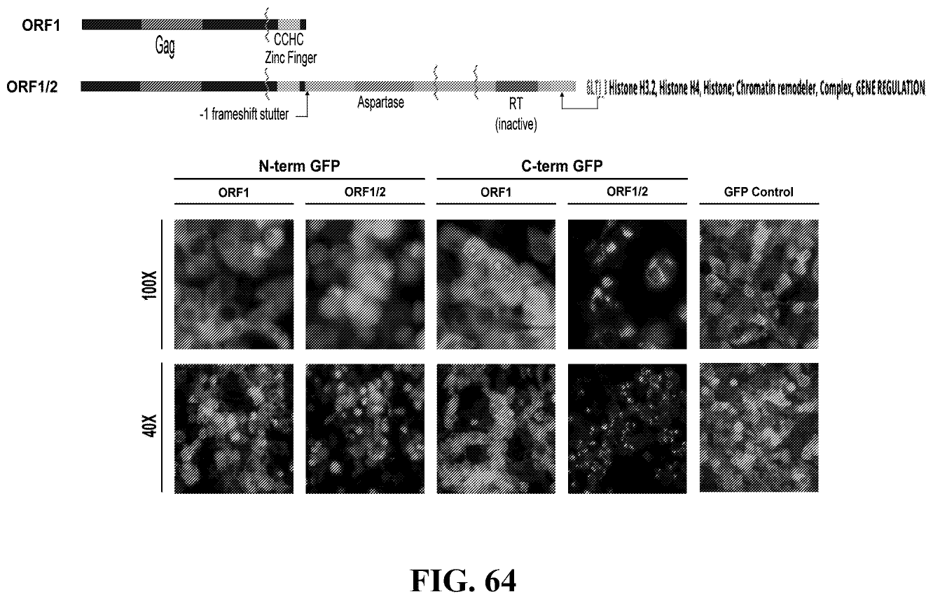

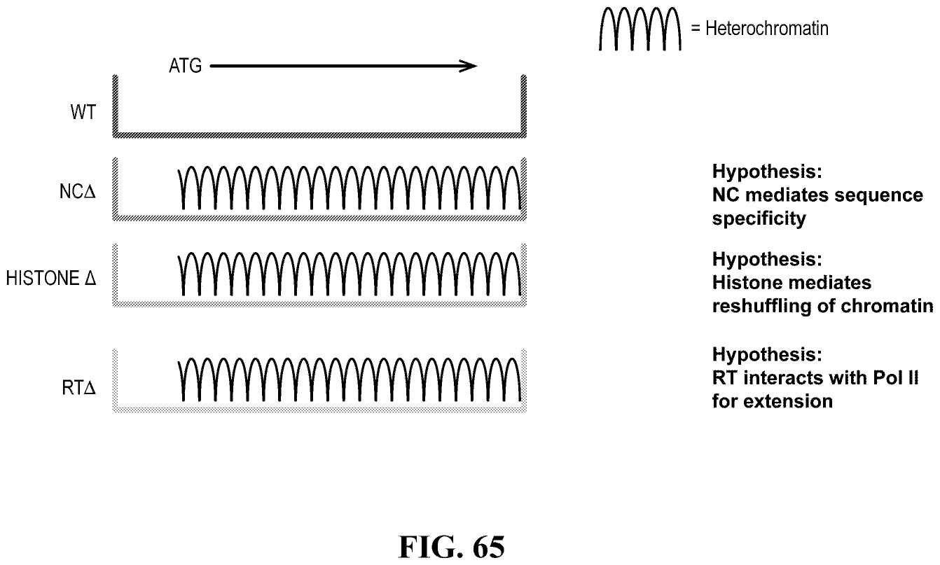

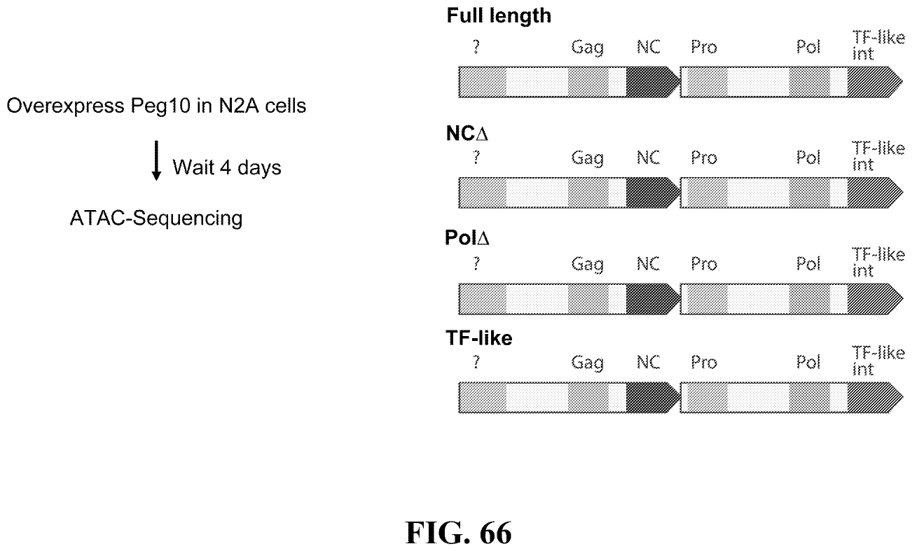

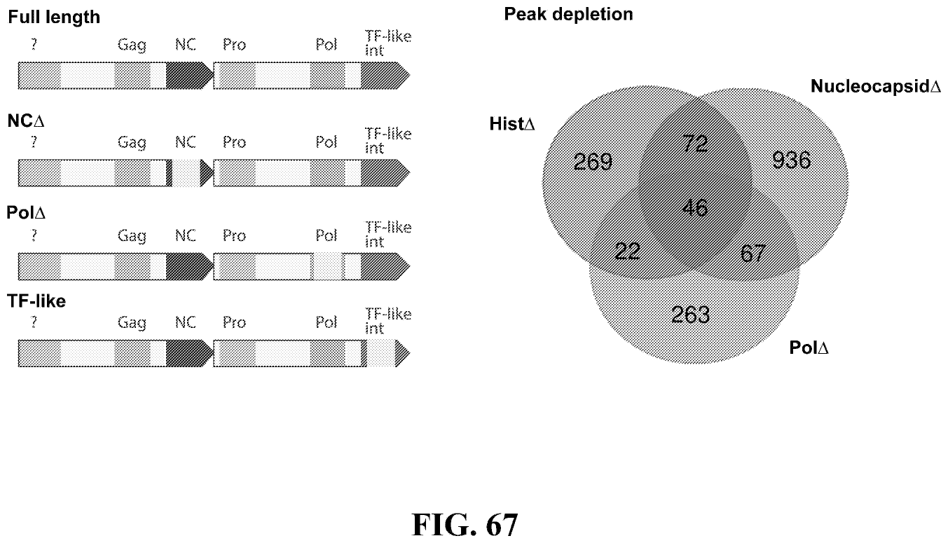

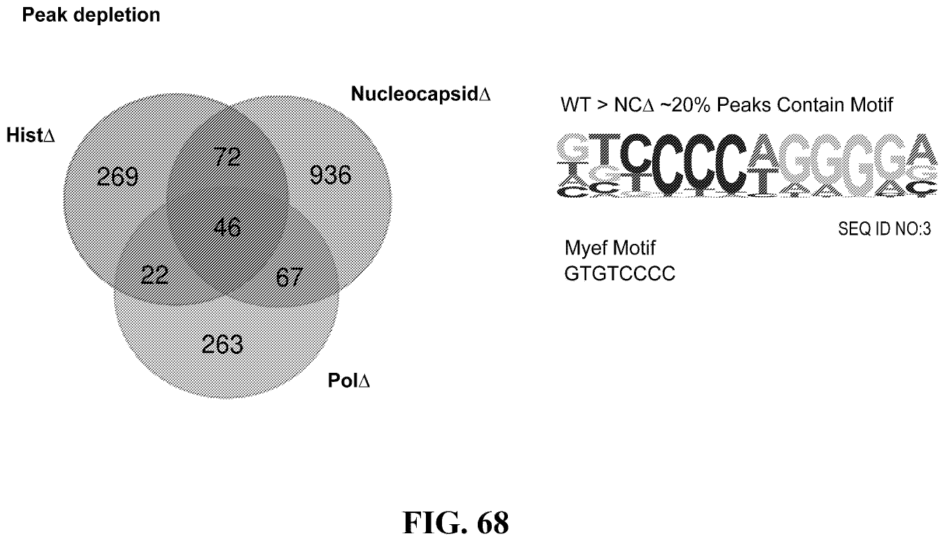

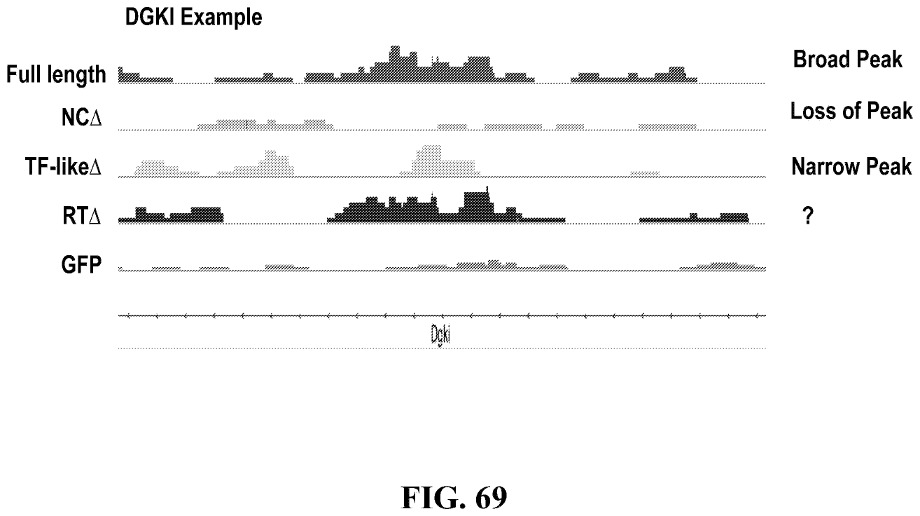

As any person of skill in the art would appreciate, any of the systems described herein can be further engineered to a minimal set of components and be applied to any suitable endogenous element. As described in Examples 3 and 4 and FIGS. 56-70, use of PEG10 is just an example approach that can be followed with any other endogenous element.

### Retroviral Env Protein

Env is a retroviral gene that encodes the protein that forms the viral envelope. The expression of the env gene allows retroviruses to target and attach to specific cell types, and to infiltrate the target cell membrane. The structure and sequence of several different env genes suggests that Env proteins are type 1 fusion machines. Type 1 fusion machines initially bind a receptor on the target cell surface, which triggers a conformational change, allowing for binding of the fusion protein. The fusion peptide inserts itself in the host cell membrane and brings the host cell membrane very close to the viral membrane, allowing for membrane fusion. The sequence of the env gene may differ significantly between retroviruses, however, the gene is always located downstream of gag, pro, and pol. The env mRNA has to be spliced to be expressed.

Env not only mediates virus entry into cells, but is also a major target for both cellular and antibody responses. It is synthesized as a precursor molecule, gp160, which is subsequently processed into the surface subunit (SU) gp120 and the transmembrane subunit (TM) gp41 by a cellular protease, and exists as a trimer of gp120-gp41 heterodimers on viral or cell membranes. The SU protein domain determines the tropism of the virus because it is responsible for the receptor-binding function of the virus. The SU domain therefore determines the specificity of the virus for a single receptor molecule. gp120 interacts with receptor and coreceptor molecules for HIV and mediates virus attachment to the cell, while gp41 causes subsequent fusion between viral and cell membranes for releasing viral core components into the cell during the initial infection process. The TM protein consists of three distinct domains: the extracellular domain, the transmembrane domain, and the cytoplasmic domain.

In some embodiments, the retroviral envelope protein is endogenous.

In some embodiments, the envelope protein may be from a Gammaretrovirus. In some embodiments, the envelope protein may be from a Deltaretrovirus.

In some embodiments, the envelope protein may be selected from, but is not necessarily limited to, envH1, envH2, envH3, envK1, envK2_1, envK2_2, envK3, envK4, envK5, envK6, envT, envW, envW1, envfrd, envR(b), envR, envF(c)2, or envF(c)1.

In an aspect, the invention provides for introduction of an RNA sequence into a transcript recruitment sequence that forms a loop secondary structure and binds to an adapter protein. In an aspect the invention provides a herein-discussed composition, wherein the insertion of distinct RNA sequence(s) that bind to one or more adaptor proteins is an aptamer sequence. In an aspect the invention provides a herein-discussed composition, wherein the aptamer sequence is two or more aptamer sequences specific to the same adaptor protein. In an aspect the invention provides a herein-discussed composition, wherein the aptamer sequence is two or more aptamer sequences specific to a different adaptor protein. In an aspect the invention provides a herein-discussed composition, wherein the adaptor protein comprises MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, PRR1. In an aspect the invention provides a herein-discussed composition, wherein the cell is a eukaryotic cell. In an aspect the invention provides a herein-discussed composition, wherein the eukaryotic cell is a mammalian cell, optionally a mouse cell. In an aspect the invention provides a herein-discussed composition, wherein the mammalian cell is a human cell. Aspects of the invention encompass embodiments relating to MS2 adaptor proteins described in Konermann et al. “Genome-scale transcriptional activation by an engineered CRISPR-Cas9 complex” Nature. 2014 Dec. 10. doi: 10.1038/nature14136, the contents of which are herein incorporated by reference in its entirety.

In some embodiments, the adaptor protein domain is an RNA-binding protein domain. The RNA-binding protein domain recognises corresponding distinct RNA sequences, which may be aptamers. For example, the MS2 RNA-binding protein recognises and binds specifically to the MS2 aptamer (or vice versa).

Similarly, an MS2 variant adaptor domain may also be used, such as the N55 mutant, especially the N55K mutant. This is the N55K mutant of the MS2 bacteriophage coat protein (shown to have higher binding affinity than wild type MS2 in Lim, F., M. Spingola, and D. S. Peabody. “Altering the RNA binding specificity of a translational repressor.” Journal of Biological Chemistry 269.12 (1994): 9006-9010).

In some embodiments, the envelope protein may comprise a cargo-binding domain. In some embodiments, the cargo-binding domain is a hairpin loop-binding element. In some embodiments, the hairpin loop-binding element is an MS2 aptamer.

In some embodiments, the retroviral gag protein and the retroviral envelope protein are both endogenous. In some embodiments, the gag protein is endogenous and the envelope protein is of viral origin. In some embodiments, the envelope protein is endogenous and the gag protein is of viral origin.

### Capture Moieties

In some embodiments, the vesicles comprise one or more capture moieties, e.g., for packaging a cargo and/or recruiting specific cargo(s) into the vesicle.

The term “nucleic acid capture moiety” or simply “capture moiety”, as used herein, refers to a moiety which binds selectively to a target molecule. Optionally, the moiety can be immobilized on an insoluble support, as in a microarray or to microparticles, such as beads. When used as a primer, a probe of the invention would likely not be anchored to a solid support. A capture moiety can “capture” a target molecule by hybridizing to the target and thereby immobilizing the target. In cases wherein the moiety itself is immobilized, the target too becomes immobilized. Such binding to a solid support may be through a linking moiety, which is bound to either the capture moiety or to the solid support.

The capture moiety may comprise one or more genes endogenous to the polynucleotide or plasmid, for example genes capable of recruiting the plasmid into the vesicle. The capture moiety may comprise exogenous genes or may comprise molecules capable of nrecruiting or capturing cargo molecules for the vesicles. In some examples, the capture moieties may interact with the cargo. The capture moieties may be nucleic acid-binding molecules, e.g., DNA, RNA, DNA-binding proteins, RNA-binding proteins, or a combination thereof. In some embodiments, the capture moieties may be protein-binding molecules, e.g., DNA, RNA, antibodies, nanobodies, antigens, receptors, ligands, fragments thereof, or a combination thereof. The capture moieties can be fused to endogenous genes or exogenous genes.

In some embodiments, the one or more capture moieties comprise DNA-binding moieties, RNA-binding moieties, protein-binding moieties, or a combination thereof.

In certain embodiments, the capture moiety may be labelled, as with, e.g., a fluorescent moiety, a radioisotope (e.g., ³²P), an antibody, an antigen, a lectin, an enzyme (e.g., alkaline phosphatase or horseradish peroxidase, which can be used in calorimetric methods), chemiluminescence, bioluminescence or other labels well known in the art. In certain embodiments, binding of a target strand to a capture moiety can be detected by chromatographic or electrophoretic methods. In embodiments in which the capture moiety does not contain a detectable label, the target nucleic acid sequence may be so labelled, or, alternatively, labelled secondary probes may be employed. A “secondary probe” includes a nucleic acid sequence which is complementary to either a region of the target nucleic acid sequence or to a region of the capture moiety. Region G of a probe (which will most often not be complementary to the target), might be useful in capturing a secondary labelled nucleic acid probe.

In some embodiments, the capture moiety is a nucleic acid hairpin. The terms “nucleic acid hairpin”, “hairpin capture moiety”, or simply “hairpin”, as used herein, refer to a unimolecular nucleic acid-containing structure which comprises at least two mutually complementary nucleic acid regions such that at least one intramolecular duplex can form. Hairpins are described in, for example, Cantor and Schimmel, “Biophysical Chemistry”, Part III, p. 1183 (1980). In certain embodiments, the mutually complementary nucleic acid regions are connected through a nucleic acid strand; in these embodiments, the hairpin comprises a single strand of nucleic acid. A region of the capture moiety which connects regions of mutual complementarity is referred to herein as a “loop” or “linker”. In some embodiments, a loop comprises a strand of nucleic acid or modified nucleic acid. In some embodiments, the linker is not a hydrogen bond. In other embodiments, the loop comprises a linker region which is not nucleic-acid-based; however, capture moieties in which the loop region is not a nucleic acid sequence are referred to herein as hairpins. Examples of non-nucleic-acid linkers suitable for use in the loop region are known in the art and include, for example, alkyl chains (see, e.g., Doktycz et al. (1993) Biopolymers 33:1765). While it will be understood that a loop can be a single-stranded region of a hairpin, for the purposes of the discussion below, a “single-stranded region” of a hairpin refers to a non-loop region of a hairpin. In embodiments in which the loop is a nucleic acid strand, the loop preferably comprises 2-20 nucleotides, more preferably 3-8 nucleotides. The size or configuration of the loop or linker is selected to allow the regions of mutual complementarity to form an intramolecular duplex. In preferred embodiments, hairpins useful in the present invention will form at least one intramolecular duplex having at least 2 base-pairs, more preferably at least 4 base-pairs, and still more preferably at least 8 base-pairs. The number of base-pairs in the duplex region, and the base composition thereof can be chosen to assure any desired relative stability of duplex formation. For example, to prevent hybridization of non-target nucleic acids with the intramolecular duplex-forming regions of the hairpin, the number of base-pairs in the intramolecular duplex region will generally be greater than about 4 base-pairs. The intramolecular duplex will generally not have more than about 40 base-pairs. In preferred embodiments, the intramolecular duplex is less than 30 base-pairs, more preferably less than 20 base-pairs in length.

A hairpin may be capable of forming more than one loop. For example, a hairpin capable of forming two intramolecular duplexes and two loops is referred to herein as a “double hairpin”. In preferred embodiments, a hairpin will have at least one single-stranded region which is substantially complementary to a target nucleic acid sequence. “Substantially complementary” means capable of hybridizing to a target nucleic acid sequence under the conditions employed. In preferred embodiments, a “substantially complementary” single-stranded region is exactly complementary to a target nucleic acid sequence. In preferred embodiments, hairpins useful in the present invention have a target-complementary single-stranded region having at least 5 bases, more preferably at least 8 bases. In preferred embodiments, the hairpin has a target-complementary single-stranded region having fewer than 30 bases, more preferably fewer than 25 bases. The target-complementary region will be selected to ensure that target strands form stable duplexes with the capture moiety. In embodiments in which the capture moiety is used to detect target strands from a large number of non-target sequences (e.g., when screening genomic DNA), the target-complementary region should be sufficiently long to prevent binding of non-target sequences. A target-specific single-stranded region may be at either the 3′ or the 5′ end of the capture moiety strand, or it may be situated between two intramolecular duplex regions (for example, between two duplexes in a double hairpin).

### Cargo Molecules

The delivery particles described herein may be used and further comprise a number of different cargo molecules for delivery. Representative cargo molecules may include, but are not limited to, nucleic acids, polynucleotides, proteins, polypeptides, polynucleotide/polypeptide complexes, small molecules, sugars, or a combination thereof. Cargoes that can be delivered in accordance with the systems and methods described herein include, but are not necessarily limited to, biologically active agents, including, but not limited to, therapeutic agents, imaging agents, and monitoring agents. A cargo may be an exogenous material or an endogenous material.

Biologically active agents include any molecule that induces an effect in a cell. Biologically active agents may be a protein, a nucleic acid, a small molecule, a carbohydrate, and a lipid. When the cargo is or comprises a nucleic acid, the nucleic acid may be a separate entity from the DNA-based carrier. In these embodiments, the DNA-based carrier is not itself the cargo. In other embodiments, the DNA-based carrier may itself comprise a nucleic acid cargo. Therapeutic agents include chemotherapeutic agents, anti-oncogenic agents, anti-angiogenic agents, tumor suppressor agents, anti-microbial agents, enzyme replacement agents, gene expression modulating agents and expression constructs comprising a nucleic acid encoding a therapeutic protein or nucleic acid. Therapeutic agents may be peptides, proteins (including enzymes, antibodies and peptidic hormones), ligands of cytoskeleton, nucleic acid, small molecules, non-peptidic hormones and the like. To increase affinity for the nucleus, agents may be conjugated to a nuclear localization sequence. Nucleic acids that may be delivered by the method of the invention include synthetic and natural nucleic acid material, including DNA, RNA, transposon DNA, antisense nucleic acids, dsRNA, siRNAs, transcription RNA, messenger RNA, ribosomal RNA, small nucleolar RNA, microRNA, ribozymes, plasmids, expression constructs, etc.

Imaging agents include contrast agents, such as ferrofluid-based MRI contrast agents and gadolinium agents for PET scans, fluorescein isothiocyanate and 6-TAMARA. Monitoring agents include reporter probes, biosensors, green fluorescent protein and the like. Reporter probes include photo-emitting compounds, such as phosphors, radioactive moieties and fluorescent moieties, such as rare earth chelates (e.g., europium chelates), Texas Red, rhodamine, fluorescein, FITC, fluo-3, 5 hexadecanoyl fluorescein, Cy2, fluor X, Cy3, Cy3.5, Cy5, Cy5.5, Cy7, dansyl, phycocrytherin, phycocyanin, spectrum orange, spectrum green, and/or derivatives of any one or more of the above. Biosensors are molecules that detect and transmit information regarding a physiological change or process, for instance, by detecting the presence or change in the presence of a chemical. The information obtained by the biosensor typically activates a signal that is detected with a transducer. The transducer typically converts the biological response into an electrical signal. Examples of biosensors include enzymes, antibodies, DNA, receptors and regulator proteins used as recognition elements, which can be used either in whole cells or isolated and used independently (D'Souza, 2001, Biosensors and Bioelectronics 16:337-353).

One or two or more different cargoes may be delivered by the delivery particles described herein.

In some embodiments, the cargo may be linked to one or more envelope proteins by a linker, as described elsewhere herein. A suitable linker may include, but is not necessarily limited to a glycine-serine linker. In some embodiments, the glycine-serine linker is (GGS)3 (SEQ ID NO: 1).

In some embodiments, the cargo comprises a ribonucleoprotein. In specific embodiments, the cargo comprises a genetic modulating agent.

As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

Also, “altered expression” as intended herein may encompass modulating the activity of one or more endogenous gene products. Accordingly, “altered expression”, “altering expression”, “modulating expression”, or “detecting expression” or similar may be used interchangeably with respectively “altered expression or activity”, “altering expression or activity”, “modulating expression or activity”, or “detecting expression or activity” or similar terms. As used herein, “modulating” or “to modulate” generally means either reducing or inhibiting the activity of a target or antigen, or alternatively increasing the activity of the target or antigen, as measured using a suitable in vitro, cellular or in vivo assay. In particular, “modulating” or “to modulate” can mean either reducing or inhibiting the (relevant or intended) activity of, or alternatively increasing the (relevant or intended) biological activity of the target or antigen, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of the inhibitor/antagonist agents or activator/agonist agents described herein.

As will be clear to the skilled person, “modulating” can also involve effecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

In some embodiments, the genetic modulating agent may comprise one or more components of a gene editing system and/or polynucleotides encoding thereof.

In some embodiments, the gene editing system may be a CRISPR-Cas system.

CRISPR Systems

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j .molce1.2015.10.008.

Class 1 Systems

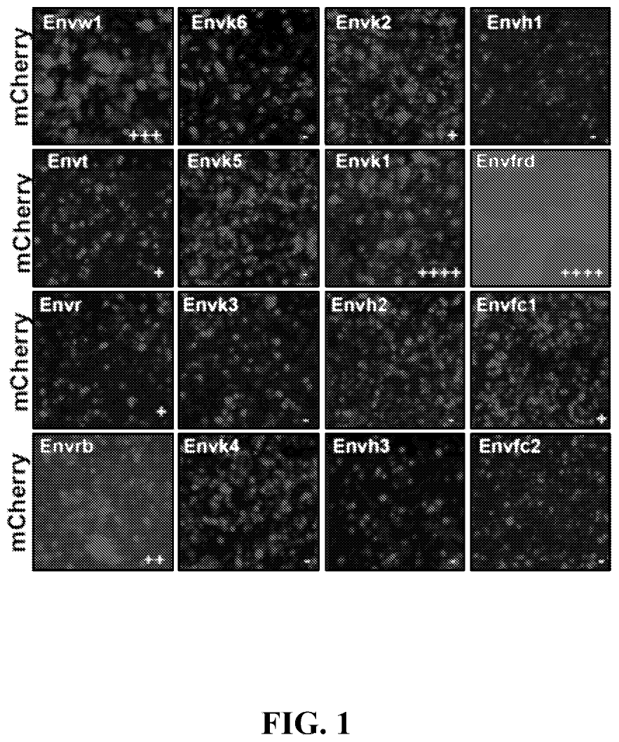

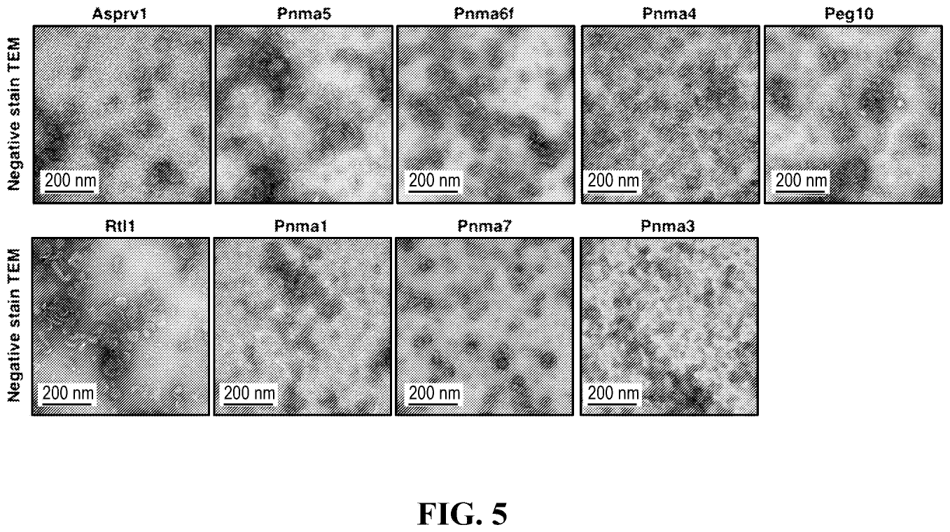

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (Feb 2020)., incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Casl, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g. Cas 5, Cas6, Cas7. RAMP proteins are characterized by having one or more RNA recognition motif domains. Large subunits (for example cas8 or cas10) and small subunits (for example, casl 1) are also typical of Class 1 systems. See, e.g., FIGS. 1 and 2. Koonin EV, Makarova KS. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1 , n5, FIG. 5.

Class 2 Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at FIG. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-Ul CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B 1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

CRISPR-Cas System Cargo Molecules

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also, the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere. Lentiviral and retroviral systems, as well as non-viral systems for delivering CRISPR-Cas system components are generally known in the art. AAV and adenovirus-based systems for CRISPR-Cas system components are generally known in the art as well as described herein (e.g. the engineered AAVs of the present invention).

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain embodiments the invention involves vectors, e.g. for delivering or introducing in a cell Cas and/or RNA capable of guiding Cas to a target locus (i.e. guide RNA), but also for propagating these components (e.g. in prokaryotic cells). This can be in addition to delivery of one or more CRISPR-Cas components or other gene modification system component not already being delivered by an engineered particle described herein. A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words, samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n⁹/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters—especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Additional effectors for use according to the invention can be identified by their proximity to casl genes, for example, though not limited to, within the region 20 kb from the start of the casl gene and 20 kb from the end of the casl gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Cas 12, Cas 12a, Cas 13a, Cas 13b, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

In other example embodiments, the Type VI RNA-targeting Cas enzyme is Cas 13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

The methods, systems, and tools provided herein may be designed for use with Class 1 CRISPR proteins, which may be Type I, Type III or Type IV Cas proteins as described in Makarova et al., The CRISPR Journal, v. 1, n., 5 (2018); DOI: 10.1089/crispr.2018.0033, incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g. Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one embodiment, Class 1 systems are characterized by the signature protein Cas3. The Cascade in particular Classl proteins can comprise a dedicated complex of multiple Cas proteins that binds pre-crRNA and recruits an additional Cas protein, for example Cash or Cas5, which is the nuclease directly responsible for processing pre-crRNA. In one embodiment, the Type I CRISPR protein comprises an effector complex comprises one or more Cas5 subunits and two or more Cas? subunits. Class 1 subtypes include Type I-A, I-B, I-C, I-U, I-D, I-E, and I-F, Type IV-A and IV-B, and Type III-A, III-D, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1 , n5, FIG. 5.

### Targeting Moieties

In some embodiments, the engineered delivery system may further comprise a targeting moiety that is capable of specifically binding to a target cell. To efficiently target a delivery vesicle to cells, such as cancer cells, it is useful that the targeting moiety have an affinity for a cell surface receptor and to link the targeting moiety in sufficient quantities to have optimum affinity for the cell surface receptors; and determining these aspects are within the ambit of the skilled artisan. In the field of active targeting, there are a number of cell-, e.g., tumor-, specific targeting ligands.

Also as to active targeting, with regard to targeting cell surface receptors such as cancer cell surface receptors, targeting ligands on liposomes can provide attachment of liposomes to cells, e.g., vascular cells, via a non-internalizing epitope; and, this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bilayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirus or AAV. Tf is a monomeric serum glycoprotein of approximately 80 KDa involved in the transport of iron throughout the body. Tf binds to the TfR and translocates into cells via receptor-mediated endocytosis. The expression of TfR can be higher in certain cells, such as tumor cells (as compared with normal cells) and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the blood—brain barrier. EGFR, is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm. With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG (SEQ ID NO:4) such as APRPG-PEG-modified. VCAM, the vascular endothelium plays a key role in the pathogenesis of inflammation, thrombosis and atherosclerosis. CAMs are involved in inflammatory disorders, including cancer, and are a logical target; E- and P-selectins, VCAM-1 and ICAMs can be used to target a lipid entity of the invention., e.g., with PEGylation. Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix. Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD. Aptamers are ssDNA or RNA oligonucleotides that impart high affinity and specific recognition of the target molecules by electrostatic interactions, hydrogen bonding and hydrophobic interactions as opposed to Watson-Crick base-pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer). The targeting moiety can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the particular environment or microenvironment such as an endocytic vacuole or the acidotic tumor mass. pH-sensitive copolymers can also be incorporated in embodiments of the invention and can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)). Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at a site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature-triggered system can employ lysolipid temperature-sensitive liposomes. The invention also comprehends redox-triggered delivery: The difference in redox potential between normal and inflamed or tumor tissues, and between the intra- and extra-cellular environments has been exploited for delivery; e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction-sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization, leading to release of payload. Calcein release from reduction-sensitive lipid entity of the invention containing a disulfide conjugate can be more useful than a reduction-insensitive embodiment. Enzymes can also be used as a trigger to release payload. Enzymes, including MMPs (e.g. MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially an engineered enzyme-sensitive lipid entity of the invention can be disrupted and release the payload. An MMP2-cleavable octapeptide (Gly-Pro-Leu-Gly-Ile-Ala-Gly-Gln) (SEQ ID NO: 5) can be incorporated into a linker, and can have antibody targeting, e.g., antibody 2C5. The invention also comprehends light-or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefore can be a benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can then be by exposure to a magnetic field.

Also as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides destabilize the endosomal membrane after the conformational transition/activation at a lowered pH. Amines are protonated at an acidic pH and cause endosomal swelling and rupture by a buffer effect. Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and, histidine-rich peptides have the ability to fuse with the endosomal membrane, resulting in pore formation, and can buffer the proton pump, causing membrane lysis.

Also as to active targeting, cell-penetrating peptides (CPPs) facilitate uptake of macromolecules through cellular membranes and, thus, enhance the delivery of CPP-modified molecules inside the cell. CPPs can be split into two classes: amphipathic helical peptides, such as transportan and MAP, where lysine residues are major contributors to the positive charge; and Arg-rich peptides, such as TATp, Antennapedia or penetratin. TATp is a transcription-activating factor with 86 amino acids that contains a highly basic (two Lys and six Arg among nine residues) protein transduction domain, which brings about nuclear localization and RNA-binding. Other CPPs that have been used for the modification of liposomes include the following: the minimal protein transduction domain of Antennapedia, a Drosophilia homeoprotein, called penetratin, which is a 16-mer peptide (residues 43-58) present in the third helix of the homeodomain; a 27-amino acid-long chimeric CPP, containing the peptide sequence from the amino terminus of the neuropeptide galanin bound via the Lys residue, mastoparan, a wasp venom peptide; VP22, a major structural component of HSV-1 facilitating intracellular transport and transportan (18-mer) amphipathic model peptide that translocates plasma membranes of mast cells and endothelial cells by both energy-dependent and -independent mechanisms. The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can deliver cargos to the mitochondrial interior via membrane fusion. A lipid entity of the invention surface-modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhance accumulation in a desired site and/or promote organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

An embodiment of the invention includes the delivery system comprising an actively targeting lipid particle or nanoparticle or liposome or lipid bilayer delivery system; or comprising a lipid particle or nanoparticle or liposome or lipid bilayer comprising a targeting moiety whereby there is active targeting or wherein the targeting moiety is an actively targeting moiety. A targeting moiety can be one or more targeting moieties, and a targeting moiety can be for any desired type of targeting such as, e.g., to target a cell such as any herein-mentioned; or to target an organelle such as any herein-mentioned; or for targeting a response such as to a physical condition such as heat, energy, ultrasound, light, pH, chemical such as enzymatic, or magnetic stimuli; or to target to achieve a particular outcome such as delivery of payload to a particular location, such as by cell penetration.

It should be understood that as to each possible targeting or active targeting moiety herein-discussed, there is an aspect of the invention wherein the delivery system comprises such a targeting or active targeting moiety. Likewise, the following table provides exemplary targeting moieties that can be used in the practice of the invention, and, as to each an aspect of the invention provides a delivery system that comprises such a targeting moiety.

TABLE 1

| Targeting Moiety | Target Molecule | Target Cell or Tissue |
|---|---|---|
| folate | folate receptor | cancer cells |
| transferrin | transferrin | cancer cells |
| | receptor | |
| Antibody CC52 | rat CC531 | rat colon adenocarcinoma |
| | | CC531 |
| anti- HER2 antibody | HER2 | HER2 -overexpressing |
| | | tumors |
| anti-GD2 | GD2 | neuroblastoma, |
| | | melanoma |
| anti-EGFR | EGFR | tumor cells overexpressing |
| | | EGFR |
| pH-dependent | | ovarian carcinoma |
| fusogenic peptide | | |
| diINF-7 | | |
| anti-VEGFR | VEGF Receptor | tumor vasculature |
| anti-CD19 | CD19 (B cell | leukemia, lymphoma |
| | marker) | |
| cell-penetrating | | blood-brain barrier |
| peptide | | |
| cyclic arginine- | avβ3 | glioblastoma cells, human |
| glycine-aspartic | | umbilical vein endothelial |
| acid-tyrosine- | | cells, tumor angiogenesis |
| cysteine peptide | | |
| (c(RGDyC)-LP) (SEQ | | |
| ID NO: 6) | | |
| ASSHN peptide | | endothelial progenitor cells; |
| | | anti-cancer |
| PR_b peptide | α5β1 integrin | cancer cells |
| AG86 peptide | α6β4 integrin | cancer cells |
| KCCYSL (P6.1 | HER-2 receptor | cancer cells |
| peptide) (SEQ ID | | |
| NO: 7) | | |
| affinity peptide LN | Aminopeptidase N | APN-positive tumor |
| (YEVGHRC) (SEQ | (APN/CD13) | |
| ID NO: 8) | | |
| synthetic somatostatin | Somatostatin | breast cancer |
| analogue | receptor 2 (SSTR2) | |
| anti-CD20 monoclonal | B-lymphocytes | B cell lymphoma |
| antibody | | |

Thus, in an embodiment of the delivery system, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an aspect of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference).

Moreover, in view of the teachings herein the skilled artisan can readily select and apply a desired targeting moiety in the practice of the invention as to a lipid entity of the invention. The invention comprehends an embodiment wherein the delivery system comprises a lipid entity having a targeting moiety.

In some embodiments, the target cell may be a mammalian cell. In some embodiments, the mammalian cell may be a cancer cell, as described further below.

In some embodiments, the mammalian cell may be infected with a pathogen. In some embodiments, the pathogen may be a virus, as described further below.

In some embodiments, the targeting moiety comprises a membrane fusion protein. In some embodiments, the membrane fusion protein is the G envelope protein of vesicular stomatitis virus (VSV-G).

Membrane fusion is a universal and important biological phenomenon that occurs when two separate lipid membranes merge into a single continuous bilayer. Fusion reactions share common features, but are catalyzed by diverse proteins. These proteins mediate the initial recognition of the membranes that are destined for fusion and pull the membranes close together to destabilize the lipid/water interface and to initiate mixing of the lipids. A single fusion protein may do everything or assemblies of protein complexes may be required for intracellular fusion reactions to guarantee rigorous regulation in space and time. Cellular fusion machines are adapted to fit the needs of different reactions but operate by similar principles in order to achieve merging of the bilayers.

Membrane fusion can range from cell fusion and organelle dynamics to vesicle trafficking and viral infection. Without exception, all of these fusion events are driven by membrane fusion proteins, also known as fusogens. The common fusion process mediated by fusion proteins consists of a series of steps that includes the approach of two opposing lipid membranes, breaking the lipid bilayers, and finally merging the two lipid bilayers into one. Much of our understanding of membrane fusion comes from studies of vesicle fusion, which is driven by a special kind of protein called SNARE. The SNARE proteins on vesicles (v-SNARE) and those on target membranes (t-SNARE) provide not only recognition specificity but also the energy needed for vesicle fusion.

Viral fusion is another important fusion event. Enveloped viruses that are encapsulated by membranes derived from host cells release genomes after the fusion between viral envelope and host cellular membrane. Viral fusion proteins dominate the uncoating stage. According to their structural characteristics, viral fusion proteins are classified into three types: I, II and III. Despite longstanding knowledge of viral fusion proteins, the underlying fusion mechanism remains mysterious. One such previously identified type III viral fusion protein is vesicular stomatitis virus G protein (VSV-G). Previous studies have revealed that VSV-G-triggered membrane fusion in acidic environments relies on reversible conformational changes, which return to their original state under neutral conditions. VSV-G and the fusion proteins of related rhabdoviruses (e.g., rabies virus) is the sole surface-expressed protein on the bullet-shaped virions. It mediates both attachment and low-pH-induced fusion.

### Reverse Transcriptase

In some embodiments, the system further comprises a reverse transcriptase. A reverse transcriptase (RT) is an enzyme used to generate complementary DNA (cDNA) from an RNA template, a process termed reverse transcription. Reverse transcriptases are used by retroviruses to replicate their genomes. They are also used by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses.

Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In retroviruses and retrotransposons, this cDNA can then integrate into the host genome, from which new RNA copies can be made via host-cell transcription. The same sequence of reactions is widely used in the laboratory to convert RNA to DNA for use in molecular cloning, RNA sequencing, polymerase chain reaction (PCR), or genome analysis.

The HIV reverse transcriptase also has ribonuclease activity that degrades the viral RNA during the synthesis of cDNA, as well as DNA-dependent DNA polymerase activity that copies the sense cDNA strand into an antisense DNA to form a double-stranded viral DNA intermediate (vDNA).

### Delivery Vesicles

Also envisioned within the scope of the invention is a delivery vesicle comprising one or more components encoded in the one or more polynucleotides in the engineered delivery system described herein.

As described elsewhere herein, such components include, but are not necessarily limited to, one or more polynucleotides encoding one or more endogenous retroviral elements for forming a delivery vesicle and one or more capture moieties for packaging a cargo within the delivery vesicle. The one or more endogenous retroviral elements for forming a delivery vesicle may comprise two or more of a retroviral gag protein, a retroviral envelope protein, a retroviral reverse transcriptase or a combination thereof.

In some embodiments, the retroviral gag protein may be endogenous. In some embodiments, the retroviral envelope protein may be endogenous. In some embodiments, the retroviral gag protein and the retroviral envelope protein are both endogenous. As described elsewhere herein, the retroviral gag protein may contain the NC and MA domains. In some embodiments, the retroviral gag protein may be a gag-homology protein. The gag-homology protein may be Arc1, Asprv1, PNMA1, PNMA3, PNMA4, PNMA5, PNMA6, PNMA7, PEG10, RTL1, MOAP1, or ZCCHC12.

In some embodiments, the envelope protein is from a Gammaretrovirus or a Deltaretrovirus. In some embodiments, the envelope protein is selected from envH1, envH2, envH3, envK1, envK2_1, envK2_2, envK3, envK4, envK5, envK6, envT, envW, envW1, envfrd, envR(b), envR, envF(c)2, or envF(c)1.

In some embodiments, the delivery vesicle elicits a poor immune response, as described elsewhere herein.

As described elsewhere herein, the cargo may comprise nucleic acids, proteins, a complex thereof, or a combination thereof. In specific embodiments, the cargo comprises a ribonucleoprotein. The cargo may comprise a genetic modulating agent, which comprises one or more components of a gene editing system and/or polynucleotides encoding thereof.

The gene editing system may be a CRISPR-Cas system. The CRISPR-Cas system may be a Type II, Type V, or Type VI CRISPR-Cas system, as described elsewhere herein. In specific embodiments, the Type II CRISPR-Cas system is CRISPR-Cas9, the Type V CRISPR-Cas system is CRISPR-Cas12, and the Type VI CRISPR-Cas system is CRISPR-Cas13, however, the invention is not to be limited to these embodiments.

In some embodiments, the vesicle further comprises a reverse transcriptase.

In some embodiments, the one or more capture moieties comprise DNA-binding moieties, RNA-binding moieties, protein-binding moieties, or a combination thereof.

In some embodiments, the delivery vesicle is a virus-like particle.

In some embodiments, the delivery vesicle may comprise a targeting moiety, wherein the targeting moiety is capable of specifically binding to a target cell.

In some embodiments, the cell-specific targeting moiety may comprise a membrane fusion protein. In some embodiments, the membrane fusion protein is VSV-G, as described elsewhere herein.

In some embodiments, the cell-specific targeting moiety targets a mammalian cell. In some embodiments, the mammalian cell may be a cancer cell, as described further below.

In some embodiments, the mammalian cell is infected with a pathogen. In some embodiments, the pathogen may be a virus, as described further below.

### Methods of Loading Cargo Molecules in Delivery Vesicle Systems

The cargo, which is of a size sufficiently small to be enclosed in the delivery vesicle, e.g. nucleic acids and/or polypeptides, can be introduced to cells by transduction by a viral or pseudoviral particle. Methods of packaging the cargos in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral or pseudo viral particle. After packaging in a viral particle or pseudoviral particle, the viral particles can be exposed to cells (e.g. in vitro, ex vivo, or in vivo) where the viral or pseudoviral particle infects the cell and delivers the cargo to the cell via transduction. Viral and pseudoviral particles can be optionally concentrated prior to exposure to target cells. In some embodiments, the virus titer of a composition containing viral and/or pseudoviral particles can be obtained and a specific titer can be used to transduce cells.

In some embodiments, the viral vector is configured such that when the cargo is packaged the cargo(s) is/are external to the capsid or virus particle, in the sense that the cargo is not inside the capsid (enveloped or encompassed with the capsid), but is externally exposed so that it can contact the target genomic DNA. In some embodiments, the viral vector is configured such that all the cargo(s) are contained within the capsid after packaging.

One approach for packaging cargo inside vesicles involves the use of one or more “bioreactors” which produce and subsequently secrete one or more cargo-carrying vesicles. Bioreactors may comprise cells, microorganisms, or acellular systems. A bioreactor cell is generated by administering to a cell one or more polynucleotides encoding one or more endogenous retroviral elements for forming a delivery vesicle and one or more capture moieties for packaging a cargo within the delivery vesicle. One may also administer a targeting moiety to the cell, wherein the targeting moiety is capable of specifically binding to a target cell. Accordingly, the bioreactor may be capable of producing cargo-carrying vesicles that not only deliver the biologically active RNA molecule(s) to the extracellular matrix, but also to specific cells and tissues.

In some embodiments, the cargo molecule can be a polynucleotide or polypeptide that can alone or when delivered as part of a system, whether or not delivered with other components of the system, operate to modify the genome, epigenome, and/or transcriptome of a cell to which it is delivered. Such systems include, but are not limited to, CRISPR-Cas systems. Other gene modification systems, e.g. TALENs, Zinc Finger nucleases, Cre-Lox, morpholinos, etc. are other non-limiting examples of gene modification systems whose one or more components can be delivered by the engineered AAV particles described herein.

The present invention provides nucleic acid molecules, specifically polynucleotides which, in some embodiments, encode one or more peptides or polypeptides of interest. The term “nucleic acid,” in its broadest sense, includes any compound and/or substance that comprise a polymer of nucleotides. These polymers are often referred to as polynucleotides.

Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

In some embodiments, the polynucleotides of the present invention may be circular. As used herein, “circular polynucleotides” means a single stranded circular polynucleotide which acts substantially like, and has the properties of, an RNA. The term “circular” is also meant to encompass any secondary or tertiary configuration of the circular polynucleotide.

In some embodiments, the polynucleotide includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

Vesicles formed from the bioreactors described herein may be isolated by any suitable method known in the art. For example, vesicles may include a tag that may bind an antibody or an aptamer. Vesicles may also be isolated and sorted by fluorescence-activated cell sorting (FACS) or by use of size exclusion methods.

### Methods for Delivery of Cargo Using Delivery Vesicles

Also envisioned within the scope of the invention is a method for delivering cargo to one or more cells using the delivery vesicles described herein. As described, the delivery vesicle may deliver the cargo to one or more cells of a subject.

The systems described herein may comprise one or more targeting moieties that are capable of specifically binding to a target cell. Such targeting moieties may include, but are not necessarily limited to membrane fusion proteins, antibodies, peptides, cyclic peptides, small molecules or related molecular structure capable of being directed through its binding to a target, including non-immunoglobulin scaffolds, including fibronectin, lipocalin, protein A, ankyrin, thioredoxin, and the like. In some embodiments, a membrane fusion protein may include, but is not necessarily limited to, the G envelope protein of vesicular stomatitis virus (VSV-G), herpes simplex virus 1 gB (HSV-1 gB), ebolavirus glycoprotein, members of the SNARE family of proteins, and members of the syncytin family of proteins.

In some embodiments, the cargo may comprise a therapeutic agent. The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

Target cells may include, but are not necessarily limited to, mammalian cells, cancer cells, cells that are infected with a pathogen, such as a virus, bacterium, fungus, or parasite. In some embodiments, the invention comprises delivery of cargo across the blood brain barrier. As one of skill in the art may appreciate, vesicles can be engineered to have tropism to any particular desired cell type.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

It will be appreciated that administration of therapeutic entities in accordance with the invention may be in the presence of suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman WN “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “in need of treatment”, or “in need thereof” as used herein refers to a judgment made by a caregiver (e.g. physician, nurse, nurse practitioner, or individual in the case of humans; veterinarian in the case of animals, including non-human animals) that a subject requires or will benefit from treatment. This judgment is made based on a variety of factors that are in the realm of a caregiver's experience, but that include the knowledge that the subject is ill, or will be ill, as the result of a condition that is treatable by the compounds of the invention.

As used in this context, to “treat” means to cure, ameliorate, stabilize, prevent, or reduce the severity of at least one symptom or a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The administration of compositions, agents, cells, or populations of cells, as disclosed herein may be carried out in any convenient manner including by aerosol inhalation, injection, ingestion, transfusion, implantation or transplantation. The composition may be administered to a patient subcutaneously, intradermally, intratumorally, intranodally, intramedullary, intramuscularly, intrathecally, by intravenous or intralymphatic injection, or intraperitoneally.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

Methods of administering the pharmacological compositions, including agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range lies within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases. In certain embodiments, suitable dosage ranges for intravenous administration of the agent are generally about 5-500 micrograms (μg) of active compound per kilogram (Kg) body weight. Suitable dosage ranges for intranasal administration are generally about 0.01 pg/kg body weight to 1 mg/kg body weight. In certain embodiments, a composition containing an agent of the present invention is subcutaneously injected in adult patients with dose ranges of approximately 5 to 5000 μg/human and preferably approximately 5 to 500 μg/human as a single dose. It is desirable to administer this dosage 1 to 3 times daily. Effective doses may be extrapolated from dose-response curves derived from in vitro or animal model test systems. Suppositories generally contain active ingredient in the range of 0.5% to 10% by weight; oral formulations preferably contain 10% to 95% active ingredient. Ultimately the attending physician will decide on the appropriate duration of therapy using compositions of the present invention. Dosage will also vary according to the age, weight and response of the individual patient.

Preferably, the therapeutic agent may be administered in a therapeutically effective amount of the active components. The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

In some embodiments, the therapeutic agent may comprise one or more components of a gene editing system and/or polynucleotide encoding thereof.

### EXAMPLES

### Example 1

### Pseudotyping Lentiviruses with Endogenous Retroviral Envelope Proteins

Expression of various individual env proteins was tested in HEK293T cells (FIG. 1). Best expression was achieved with Envw1, Envk1, and Envfrd (Envw2). The glycoprotein of vesicular stomatitis virus (VSV-G) is able to mediate cell attachment and induce direct fusion between membranes. Applicants compared pseudotyping efficiencies of different env proteins with lentivirus DNA. Efficient particle formation was observed with Envk1, Envw1, and Envfrd (FIG. 2).

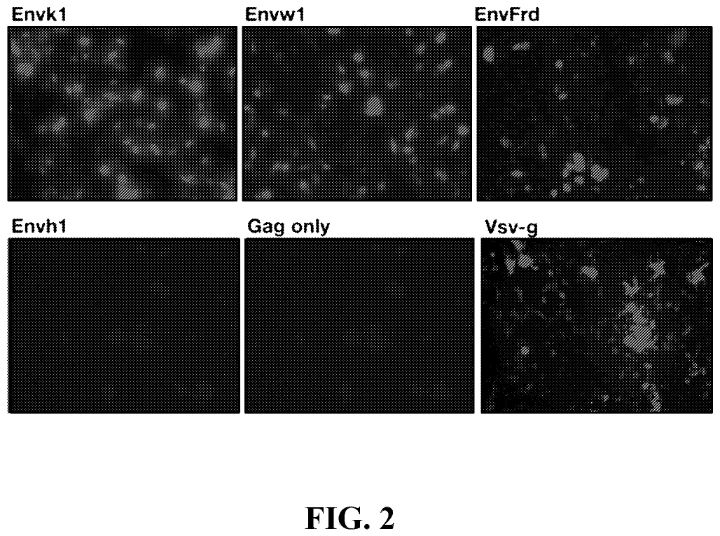

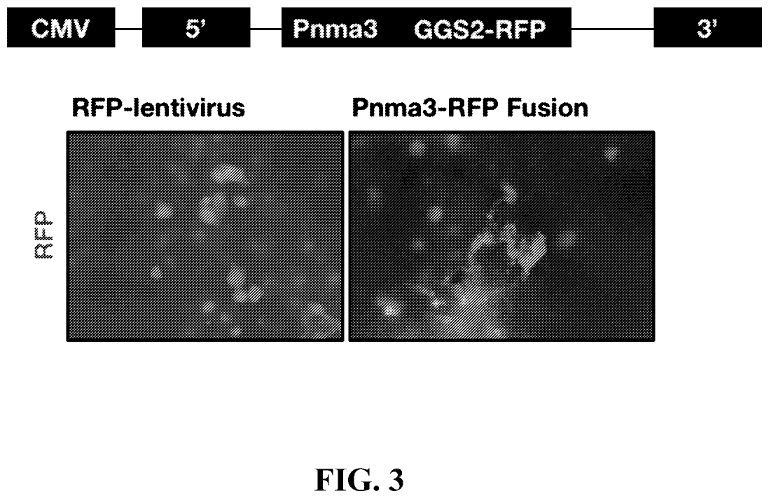

To see if the gag homology protein Pnma3 is expressed in neuronal cells, Applicants fused it to a red fluorescent reporter protein (RFP) and tested its expression in mouse and rat neurons. Results showed that expression of this fusion protein was comparable to a control RFP-lentivirus construct (FIG. 3).

### Example 2

### Screening of endogenous gag protein candidates for their ability to form capsids, secrete proteins, and transfer information

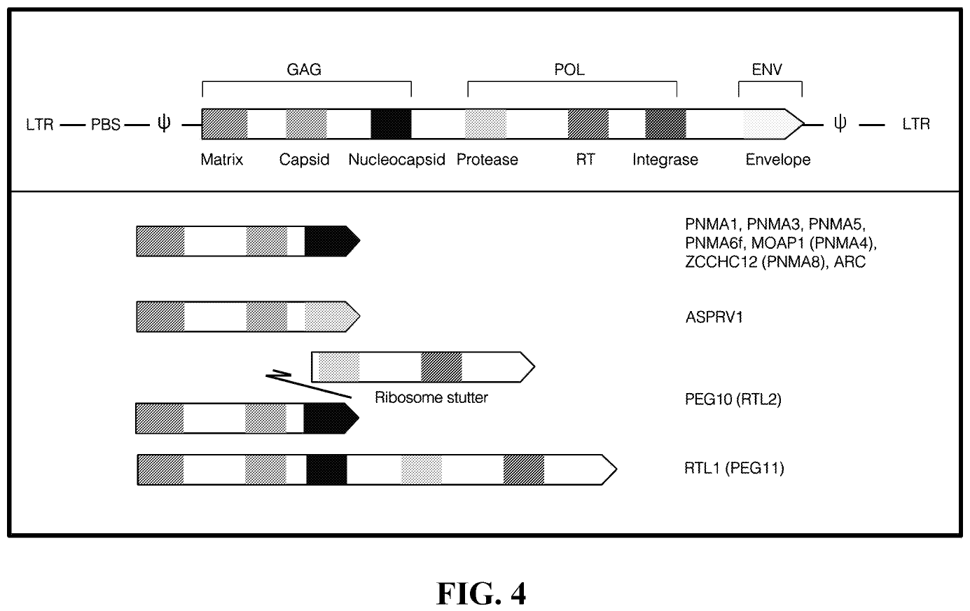

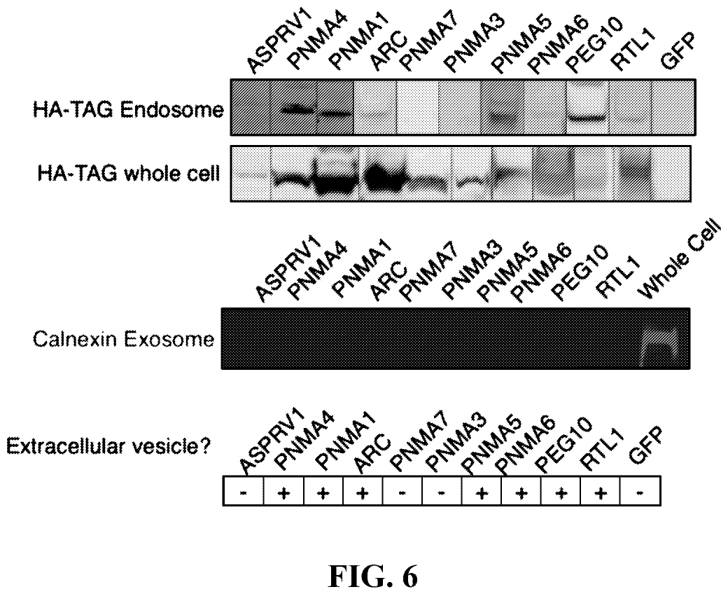

Nine endogenous gag protein candidates were identified and screened for their ability to form vesicles in vitro (FIGS. 4 and 5). Of the candidates tested, all except Asprv1 were able to form vesicles (FIG. 5 and Table 2). However, only six were able to be secreted from cells (Table 3, FIG. 6).

TABLE 2
Ability of gag protein candidates to form vesicles

| | Capsid forming in vitro? |
|---|---|
| Asprv1 | − |
| Pnma1 | + |
| Pnma3 | + |
| Pnma4 | + |
| Pnma5 | + |
| Pnma6 | + |
| Pnma7 | + |
| Peg10 | + |
| Rtl1 | + |

TABLE 3
Ability of gag protein candidates to be secreted from cells

| | Secreted Proteins? |
|---|---|
| Asprv1 | − |
| Pnma1 | + |
| Pnma3 | − |
| Pnma4 | + |
| Pnma5 | + |
| Pnma6 | + |
| Pnma7 | − |
| Peg10 | + |
| Rtl1 | + |

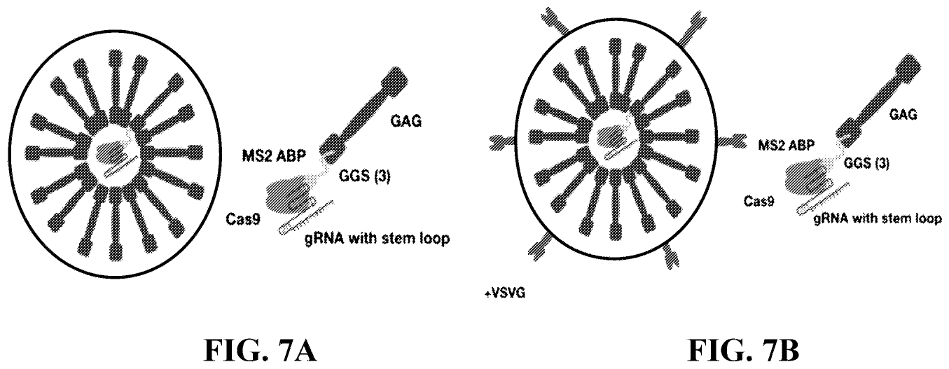

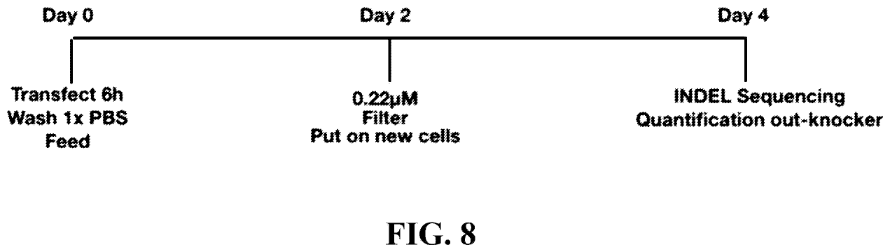

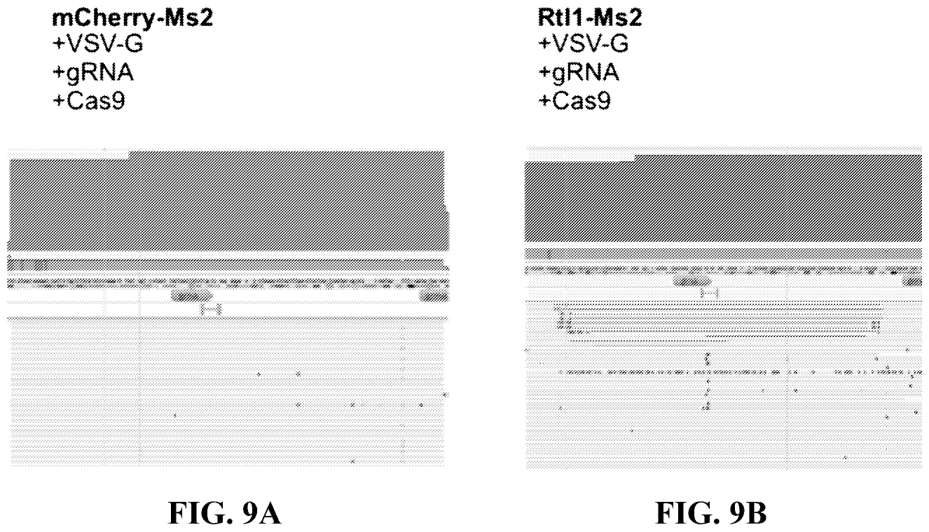

Applicants next tested the ability of the various gag protein candidates to transfer Cas9/gRNA complexes to another cell. In the absence of a membrane fusion protein (FIG. 7A), none of the candidates were able to successfully facilitate this process. However, the inclusion of VSV-G (FIG. 7B) was critical for achieving delivery of the complex to another cell (Table 4).

TABLE 4
Ability of gag protein candidates to
deliver information to a new cell

| | Transfer? |
|---|---|
| Asprv1 | − |
| Pnma1 | − |
| Pnma3 | − |
| Pnma4 | + |
| Pnma5 | − |
| Pnma6 | − |
| Pnma7 | − |
| Peg10 | + |
| Rtl1 | + |

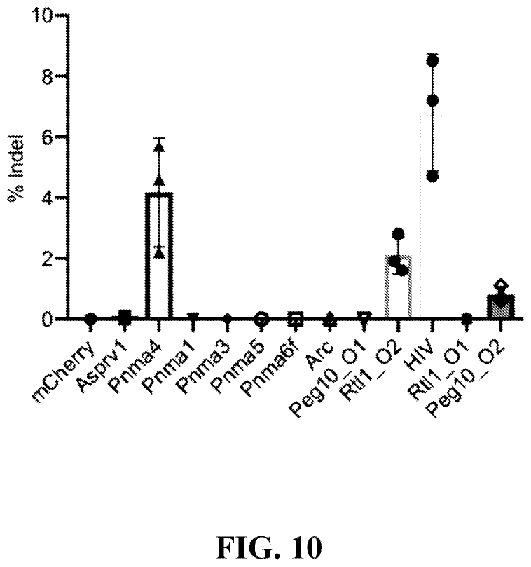

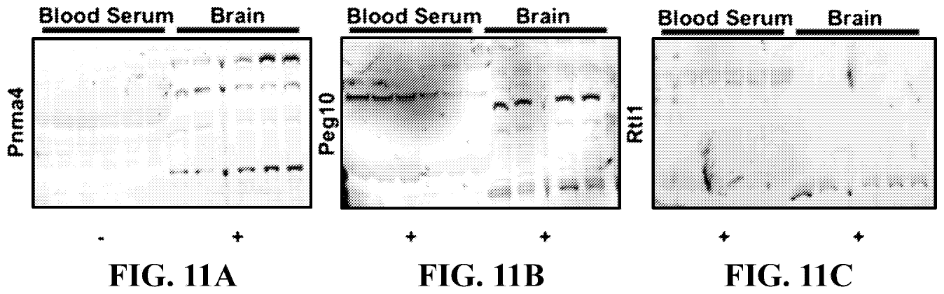

Vesicles formed using PNMA4 and RTL1 showed the highest ability to transfer gene editing complexes to new cells and induce formation of indels (FIG. 10).

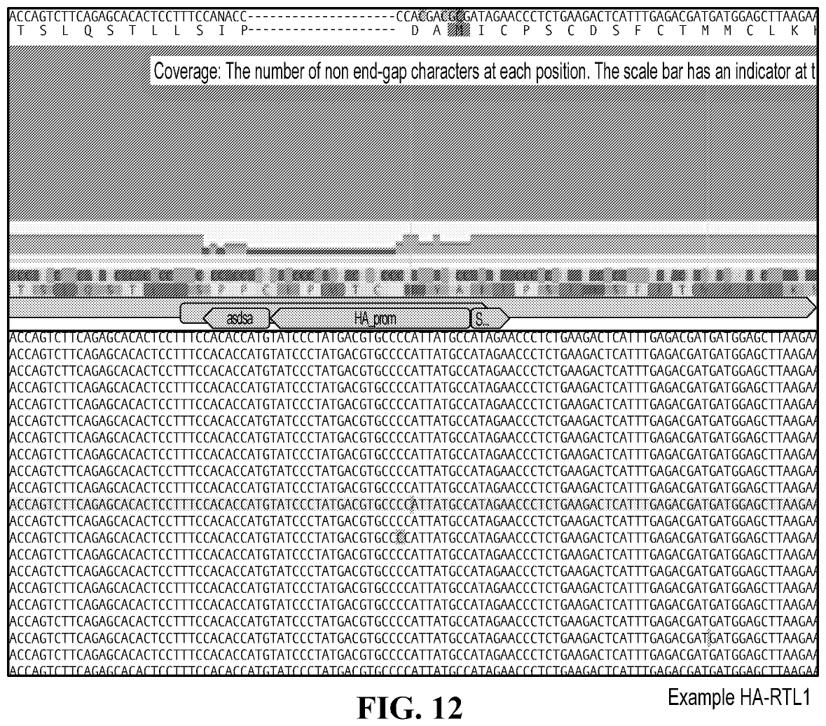

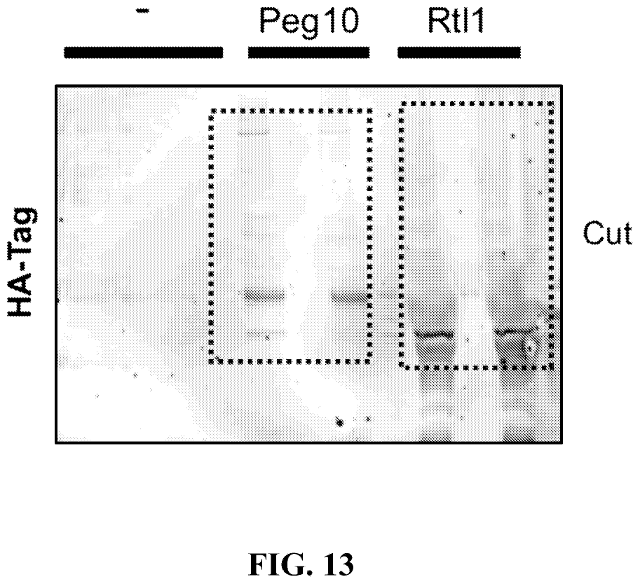

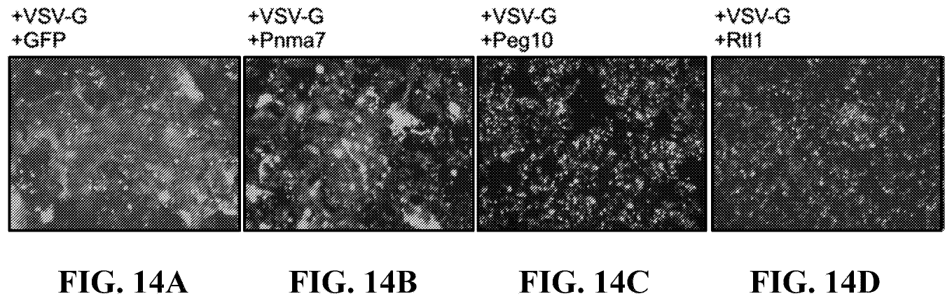

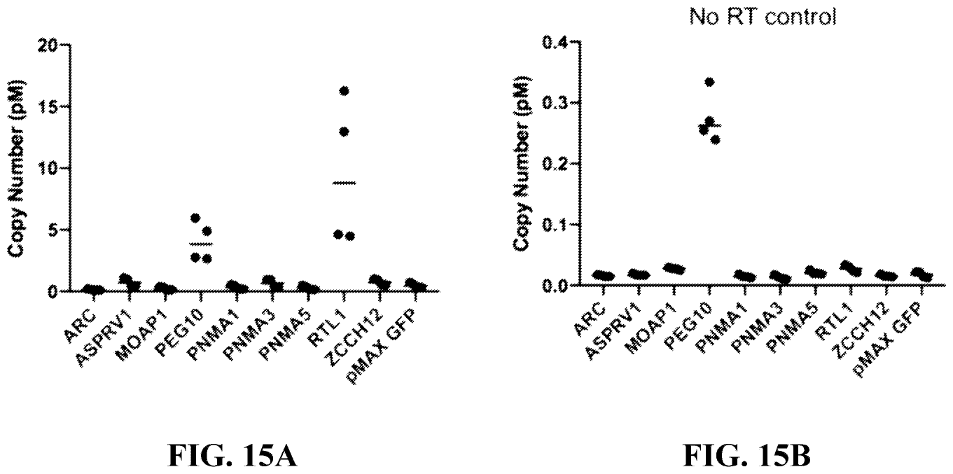

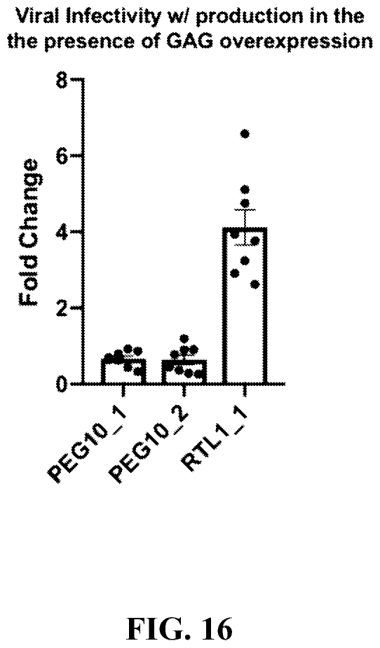

To evaluate whether gag candidates facilitated secretion from cells and subsequent transfer of information from one cell to another, Applicants also generated knock-in mice that expressed an HA-tag on endogenous gag proteins. DNA sequences encoding an exemplary HA-tagged RTL1 protein are shown in FIG. 12.

### Example 3

### Engineering an Endogenous Vector for Gene Therapy

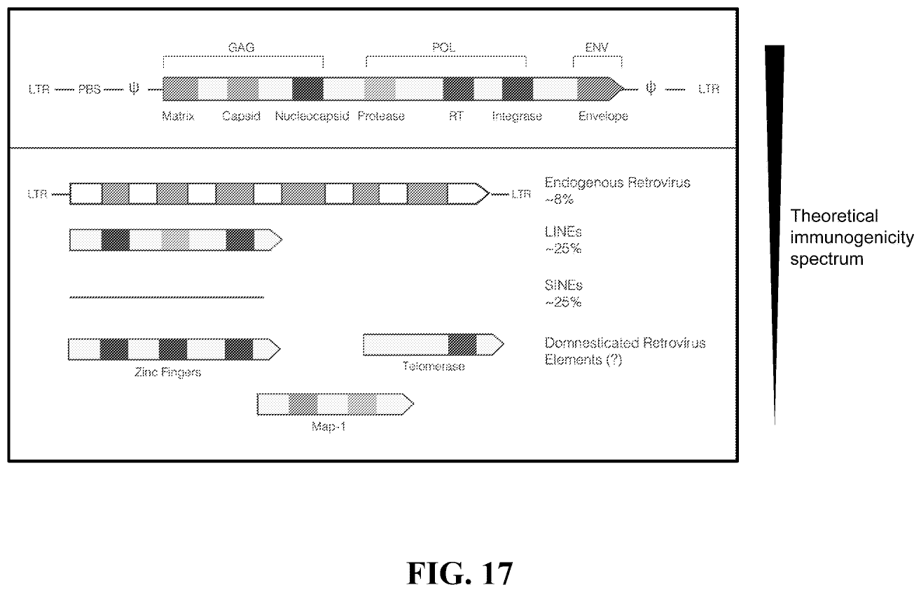

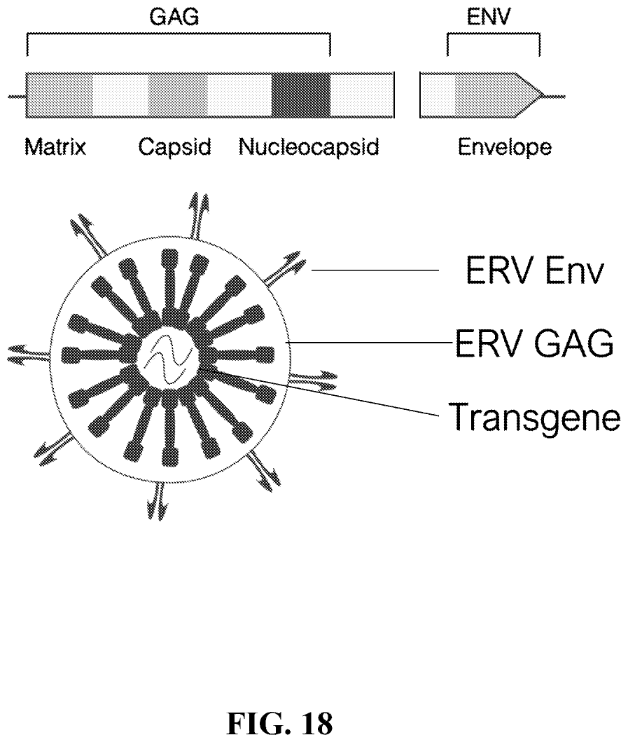

Applicants set out to create a non-immunogenic vector that can efficiently deliver gene therapies in vivo. While viral vectors are highly efficient, they can potentially be immunogenic, eliciting unwanted immune responses in target cells against the vector itself, thus rendering the therapeutic agent contained therein ineffective. Lipid nanoparticles (LNPs) are easy to produce but they have limited tropism and are typically only able to deliver about 2% of their encoded payload. Exosomes are potentially non-immunogenic but have a complicated biology and their efficacy is unclear. Applicants wanted to explore endogenous signaling systems for their potential to mediate intercellular gene transfer. For example, there are at least 40,000 coding GAGs in the human genome, with varying immunogenic potential (FIG. 17). Some highly expressed endogenous GAGs are shown in FIG. 4.

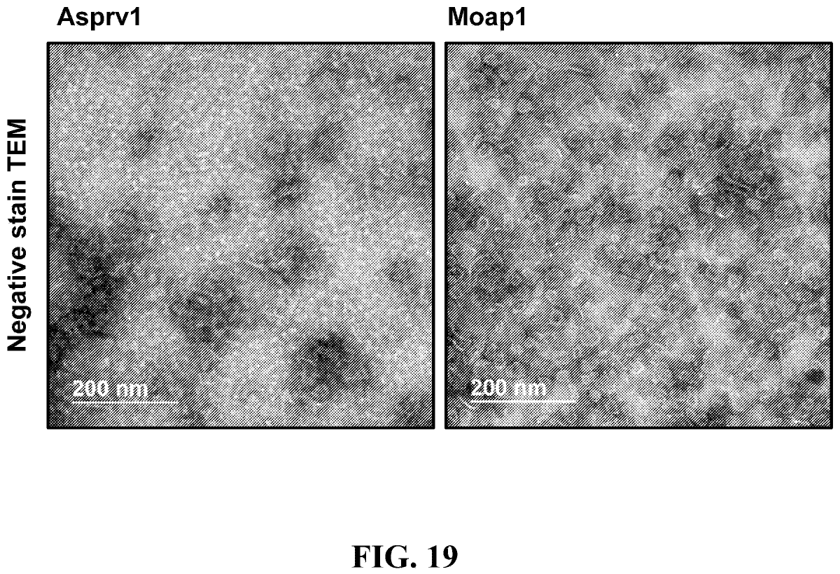

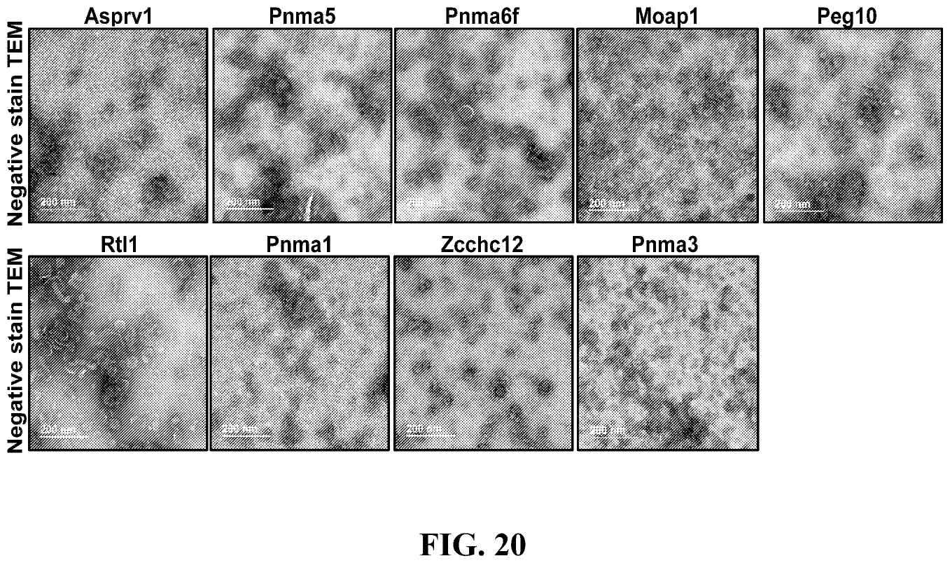

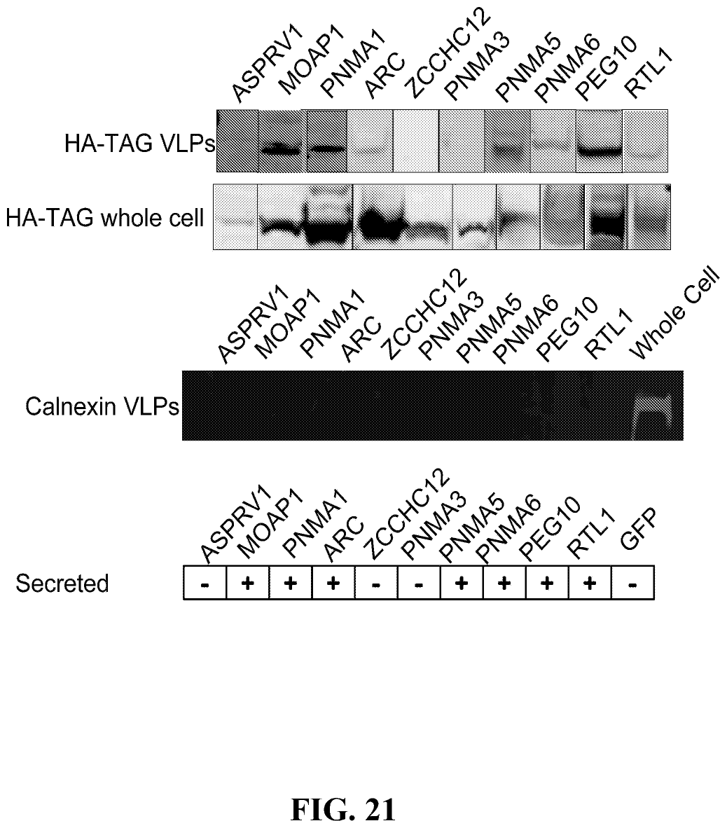

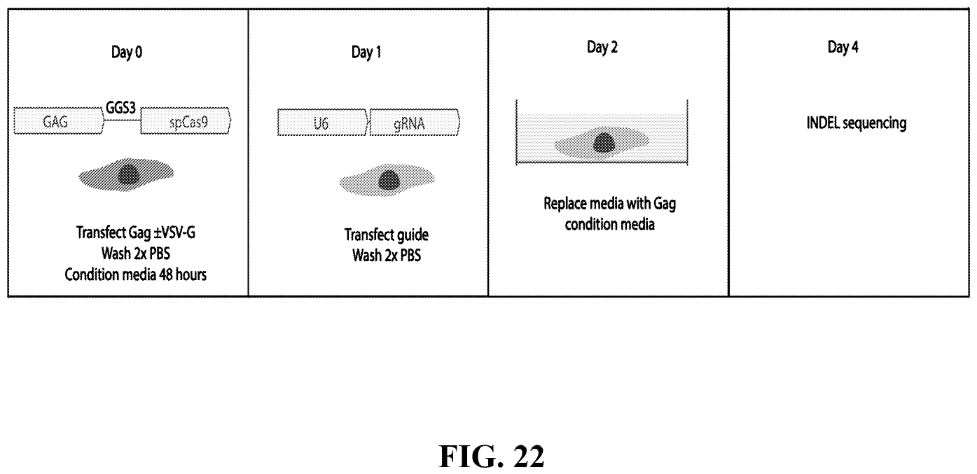

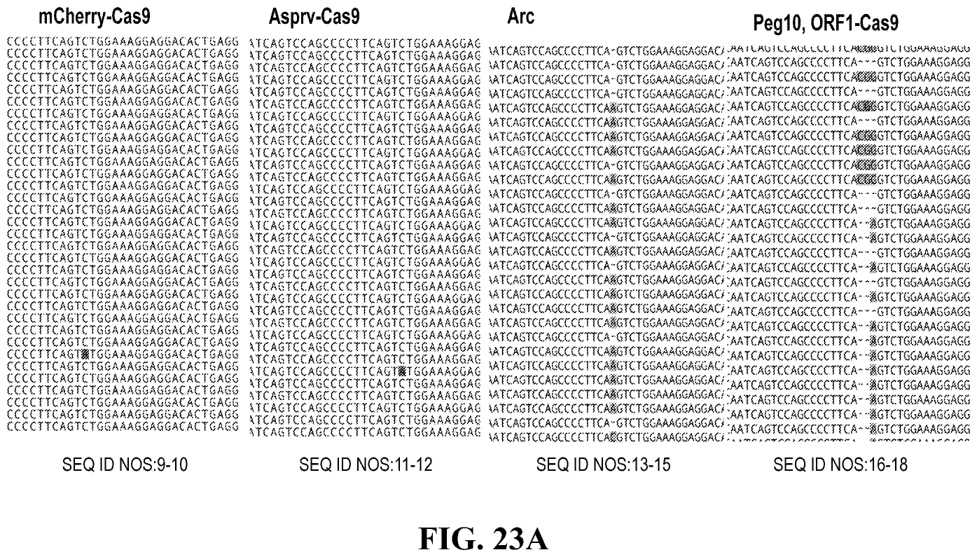

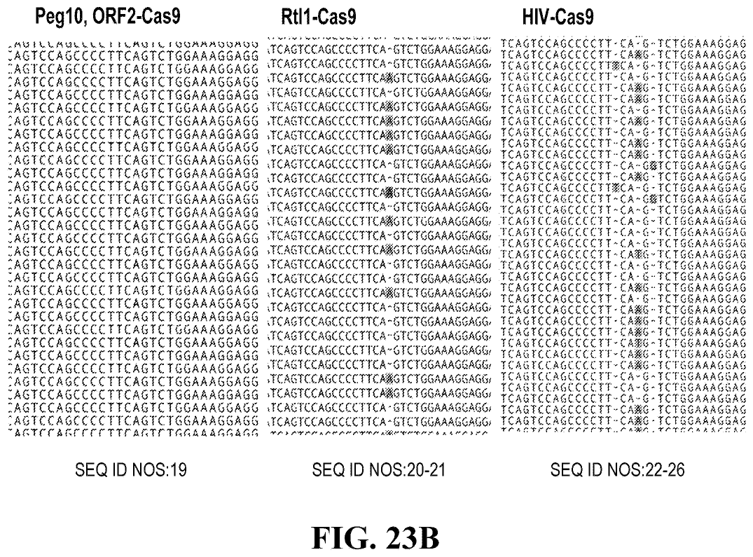

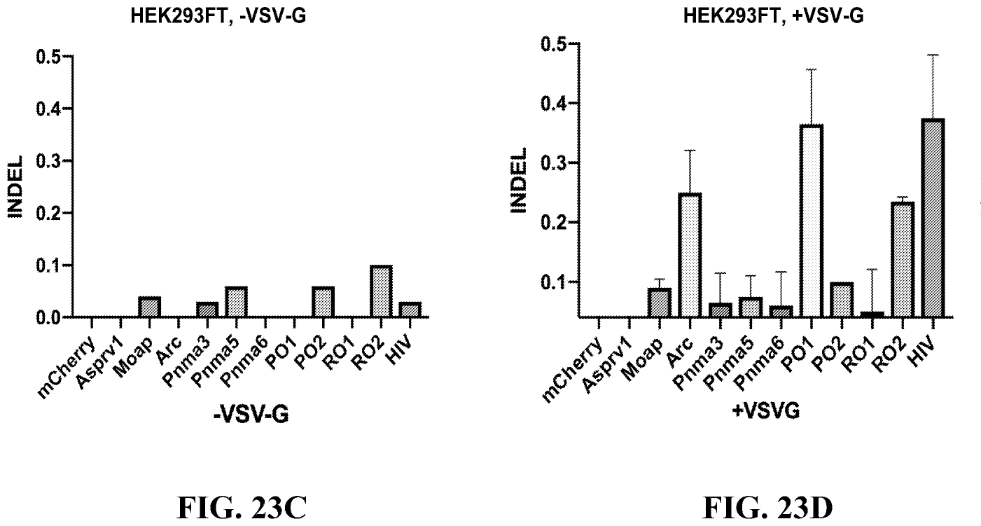

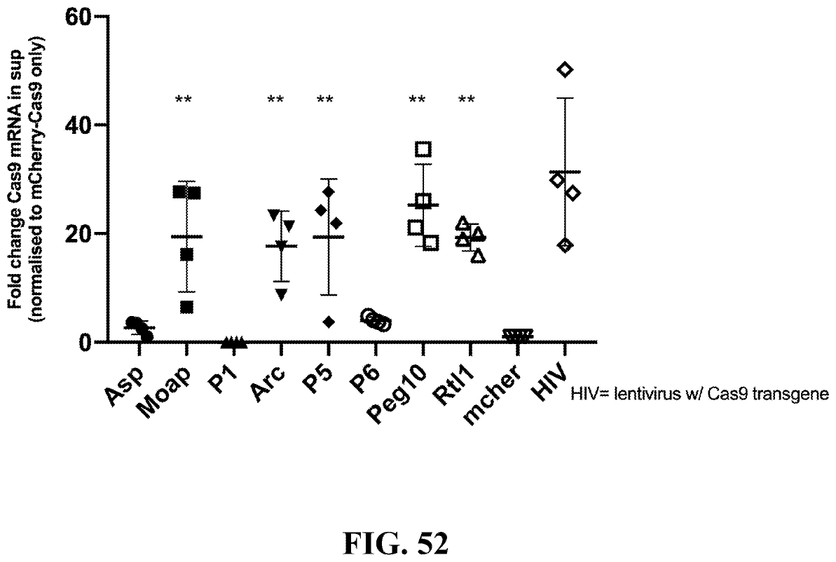

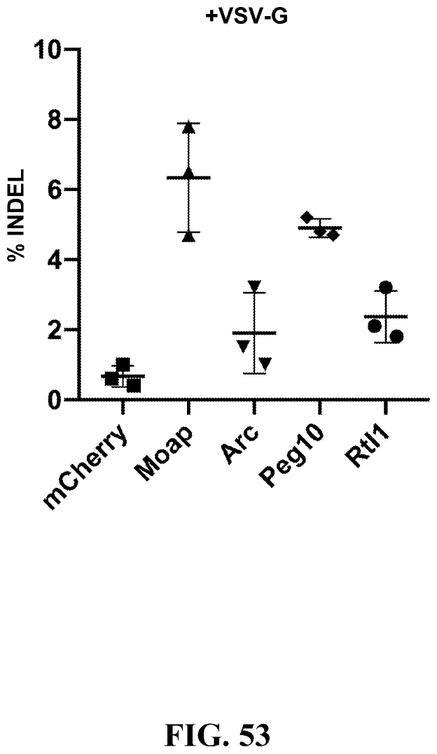

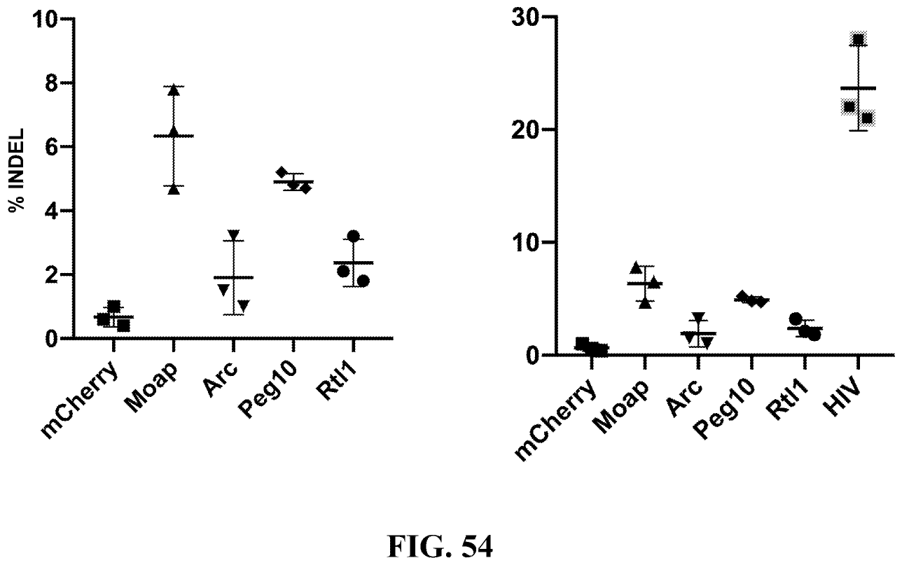

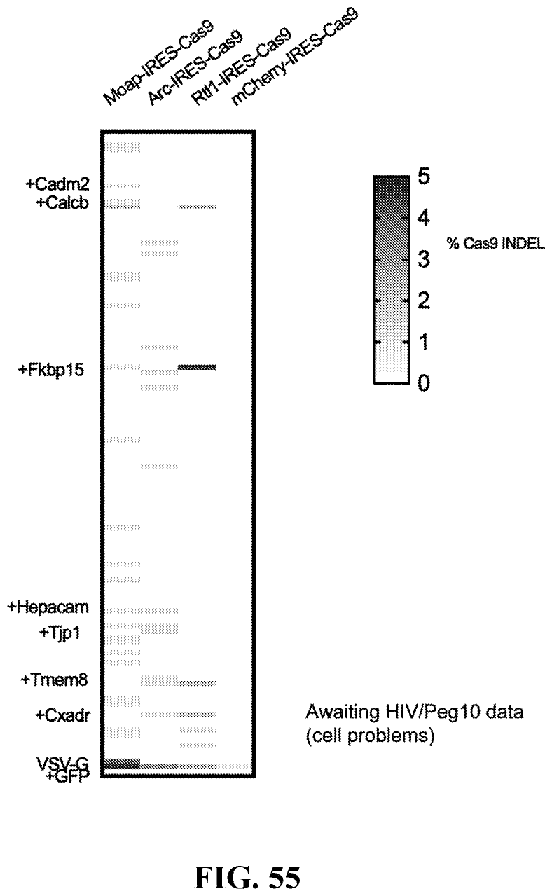

Applicants analyzed several GAGs for their ability to spontaneously form vesicles (FIGS. 19, 20). To determine which GAGs can form vesicles, HA-tagged GAGs were over-expressed in HEK cells and the supernatant was collected. The VLP fraction was centrifuged using PEG (FIG. 21). Applicants found that addition of VSV-G fusogen both improved uptake of secreted GAGs by target cells and boosted generation of INDELs (FIGS. 23A-23D, 24, 52, and 53).

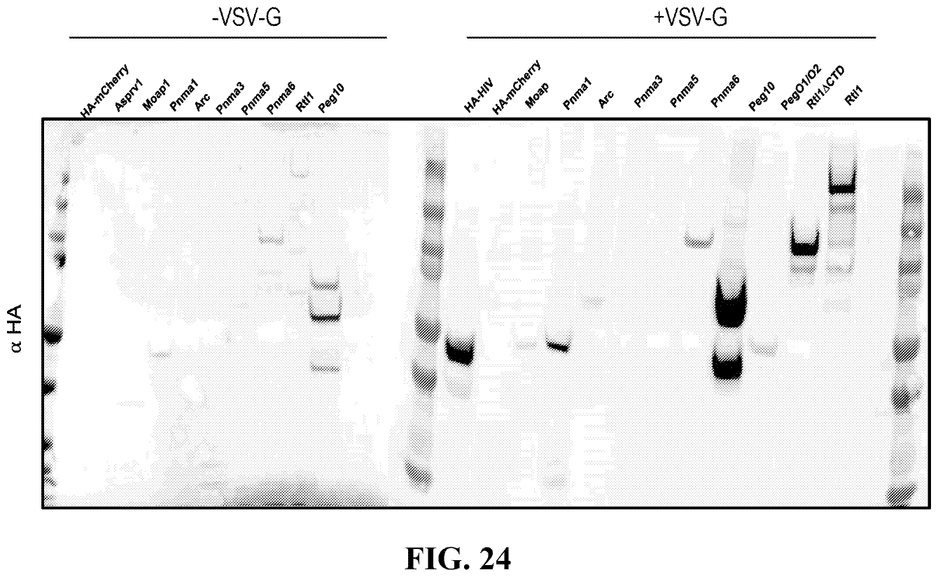

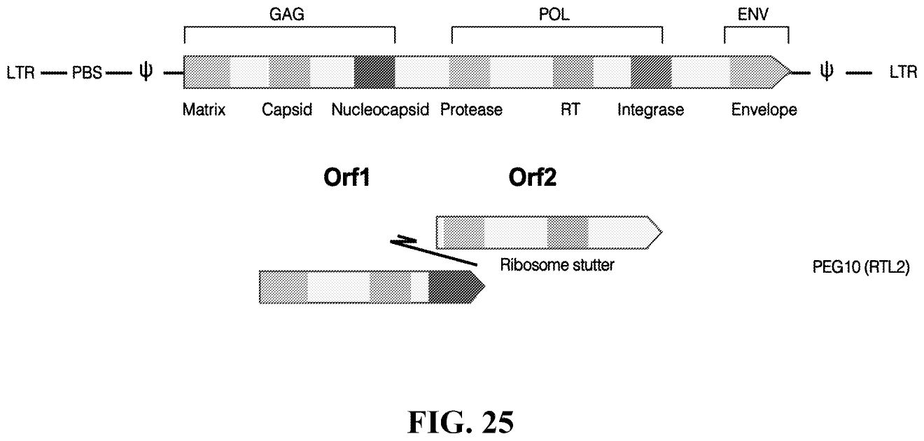

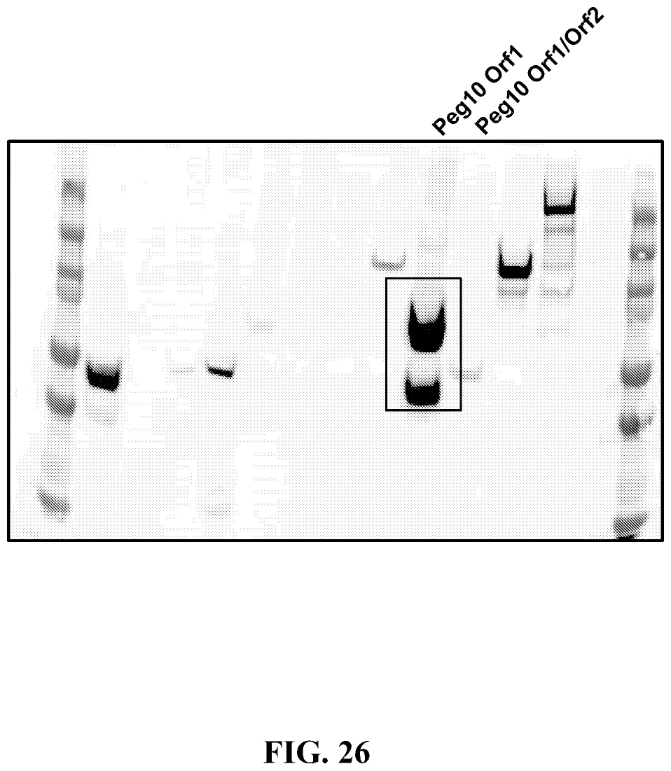

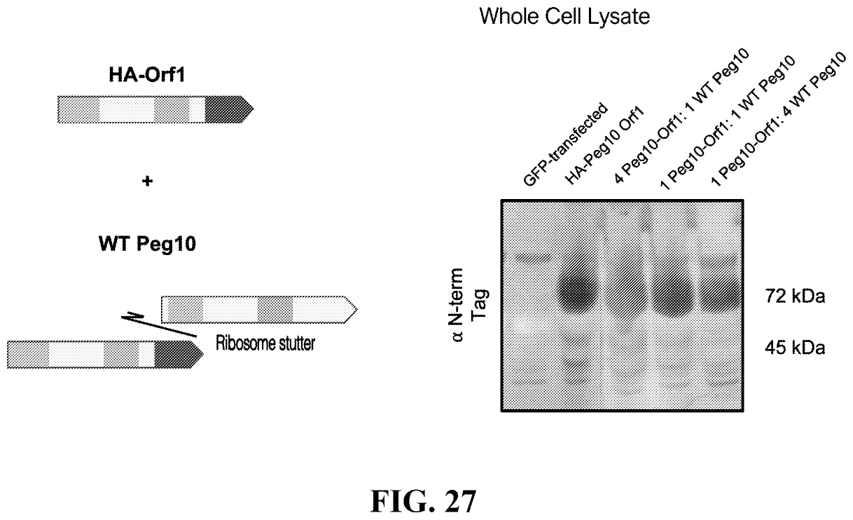

Out of all the GAGs tested, Applicants determined that PEG10 was the best candidate to mediate transfer and generate VLPs on par with HIV lentiviruses (FIG. 24). To optimize PEG10 for delivery, Applicants wanted to understand the precise biological function of PEG10, as well as the extent to which PEG10 can be reprogrammed. The gene for PEG10 includes two overlapping reading frames of the same transcript encoding distinct isoforms. The shorter isoform has a CCHC-type zinc finger motif containing a sequence characteristic of gag proteins of most retroviruses and some retrotransposons, and it functions in part by interacting with members of the TGF-beta receptor family. The longer isoform has the active-site DSG consensus sequence of the protease domain of pol proteins. The longer isoform is the result of −1 translational frameshifting that is also seen in some retroviruses (FIGS. 25, 26).

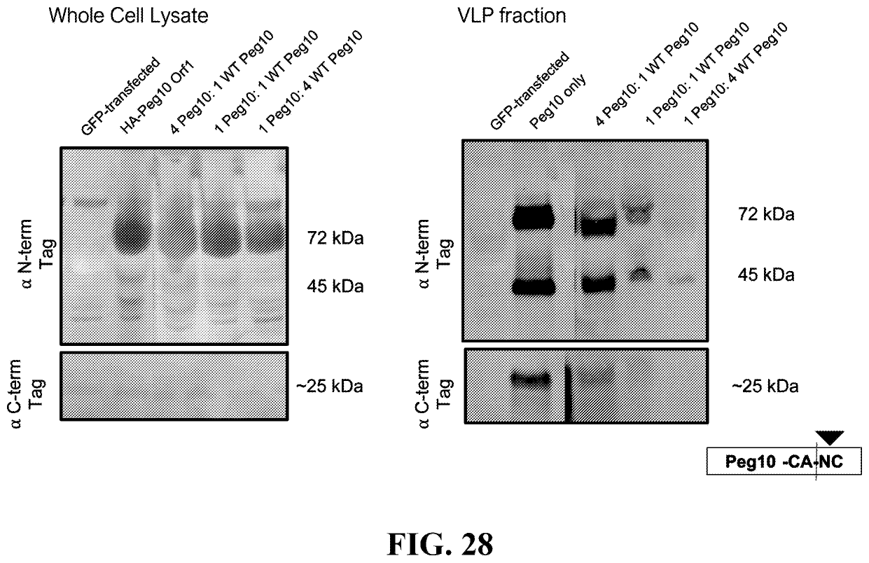

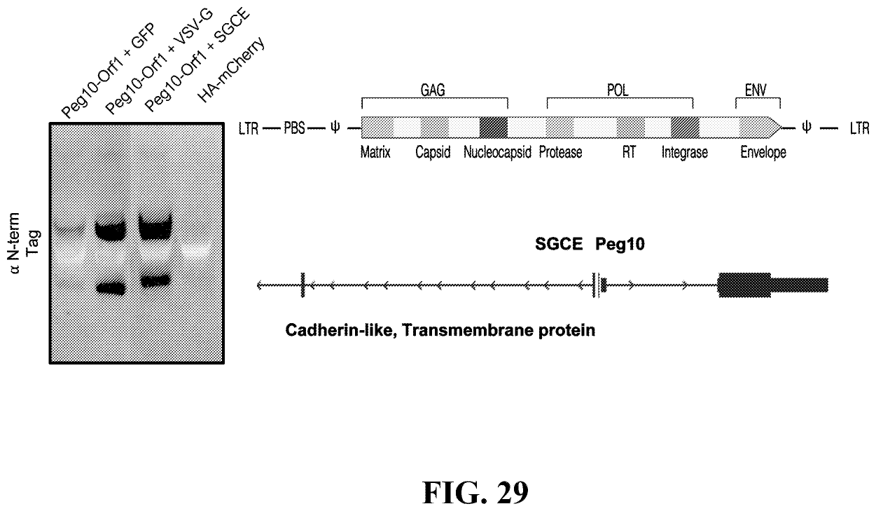

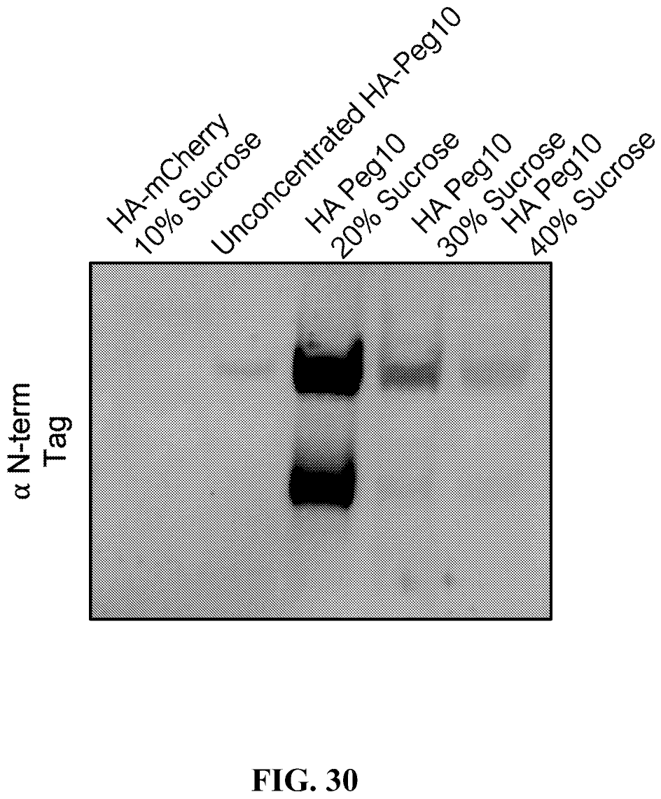

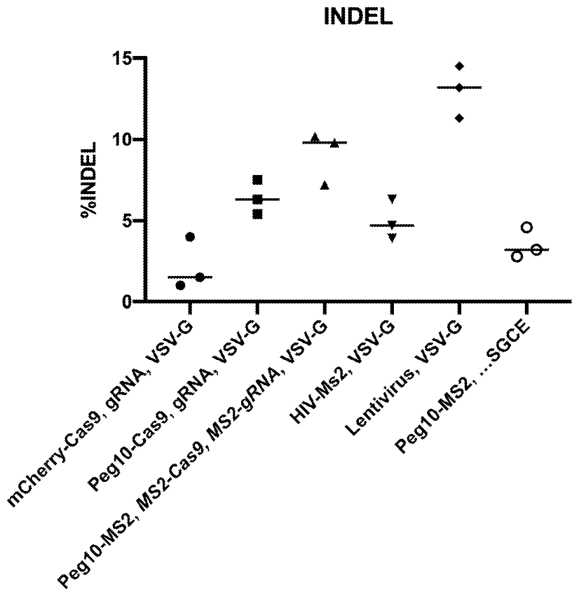

Applicants transfected cells with various PEG10 constructs and analyzed whole cell lysates and VLP fractions by immunoprecipitation. Results showed that PEG10 VLPs are processed but the protease domain is not required for this processing to occur (FIG. 28). Applicants also found that addition of VSV-G boosts PEG10 secretion and enables uptake in target cells (FIG. 29).

To boost efficiency of delivery, Applicants cultured HEK293T cells in T225 flasks. Cells were transfected with various delivery components, filtered with a 45 μm filter, and ultracentrifuged with a 20% sucrose cushion. VLPs were resuspended in 250 μL of PBS and 10 μL aliquots of the suspension were added to 20E3 cells. INDELs were then detected 48 hours later by Next Generation Sequencing (FIG. 31). These experiments revealed that PEG10 is a secreted, capsid-forming protein, and that VSV-G enables PEG10 to deliver Cas9 to target cells and mediate generation of INDELs. PEG10 VLPs are likely processed at the C-terminal domain. Applicants also found that addition of SGCE boosts PEG10 secretion but does not help boost entry (at least in HEK cells).

Applicants compiled and cloned a list of an additional 165 genes that could act as potential fusogens (Tables 5 and 6). Each of these will be evaluated individually with HIV, PEG10, Arc, and Rtl1 GAGs.

TABLE 5

| | | | | |
|---|---|---|---|---|
| Adam10 | IZUMO2 | FRMD5 | CDH3 | SPECC1 |
| ADAMDEC1 | IZUMO4 | FRMD6 | CDHR3 | Spock |
| ADGRF3 | Klrb1c | FZD6 | CLDND1 | SPOCK2 |
| AGRN | LDLRAD4 | GALNT14 | COL7A1 | ST3GAL4 |
| AGTR2 | LIMS2 | GAP43 | COPB1 | TAL1 |
| AHCY | LRP6 | GDAP1 | CXDAR | TBC1D16 |
| ALDH3B1 | LRPAP1 | GHITM | DNAJC8 | THSD4 |
| AOC3 | MAMDC2 | GHR | DOC2A | TJP2 |
| APBB1 | NCKAP1L | GNAO1 | DUSP10 | TM9SF2 |
| Arhgap32 | Nectin1 | GPM6A | DUSP15 | TMEM140 |
| ARHGAP45 | NEDD4L | GPR161 | EDA2R | Tmem68 |
| ARMCX5 | Negr1 | GRIA4 | EDNRB | Tmem8 |
| ATF5 | NLGN1 | GRID1 | EMILIN1 | TMPRSS11E |
| ATP2C2 | Nrcam | GUCY1A1 | ENPP1 | TMTC4 |
| B3GALNT1 | NRN1L | HEPACAM2 | EPHA2 | TPSAB1 |
| BAIAP2L1 | NRXN3 | IL1RAPL1 | EPHB6 | tspan11 |
| Begain | ODC1 | CD37 | ESR2 | TSPAN4 |
| TEL1L | OLFM4 | CD40 | EXTL2 | TSPAN8 |
| BIN2 | OR2T29 | CD53 | FAM198B | tspan9 |
| CACNG2 | OR5K4 | CD59 | FKBP1a | VIPR2 |
| CADM2 | OR8K1 | CD63 | FKBP15 | VOPP1 |
| CALCB | PAFAH1B1 | CD69 | FKBP2 | ZSWIM8 |
| CCDC77 | PCDH11X | CD7 | PRSS27 | SDK2 |
| CCL4L2 | PCDHGA5 | CD82 | RCAN2 | SEMA4A |
| CD160 | PDE6B | Cd9 | RCC1L | SIGLEC15 |
| CD164 | PIGV | CDC14B | RGL4 | SLA |
| CD200R1 | PIP4P1 | CDC20B | RIMS1 | SLC34A3 |
| CD247 | PLCD1 | CDC42BPB | RTBDN | SLC4A11 |
| CD248 | PLEKHB1 | CDC42SE1 | Scamp3 | SLITRK4 |
| CD2AP | PLPPR1 | CDH1 | SGCE | Snta1 |
| CD2BP2 | POMGNT1 | CDH20 | DLK1 | SNX11 |
| CD300LF | PPFIBP1 | CDH23 | CASD1 | |

TABLE 6

| | | | | |
|---|---|---|---|---|
| ADGRE5 | FBLN2 | MFAP2 | SLC27A6 | TMEM164 |
| ANXA5 | Frdm3 | MTMR4 | SLC32A1 | TMEM18 |
| ARHGAP20 | GABRA1 | NECTIN4 | SLC38A2 | tmem255a |
| ARHGAP8 | GNA11 | OLFML2B | SLC39A14 | TMEM54 |
| ATP1B1 | GNAS | OPCML | SLC4A2 | TRAF4 |
| balap3 | HTR7 | OR5B17 | SNTA1 | ZCCHC14 |
| cd63 | IL27RA | OSBPL6 | SOBP | Pnma6 |
| Cldn1 | IRS4 | Pianp | SPATA13 | |
| CLDN5 | izumo2 | PIGQ | ST3GAL4 | |
| clmp | JCAD | PLA2G12A | st8sia4 | |
| cpn2 | KIR2DL3 | PMEPA1 | TCIRG1 | |
| CRISPLD2 | KLHDC10 | PTPRB | TESC | |
| Egflam | LY6K | SLC13A5 | TGFBR3 | |
| EML6 | LYNX1 | SLC14A1 | THSD4 | |
| EXTL3 | LYPD5 | SLC22A3 | TMED8 | |

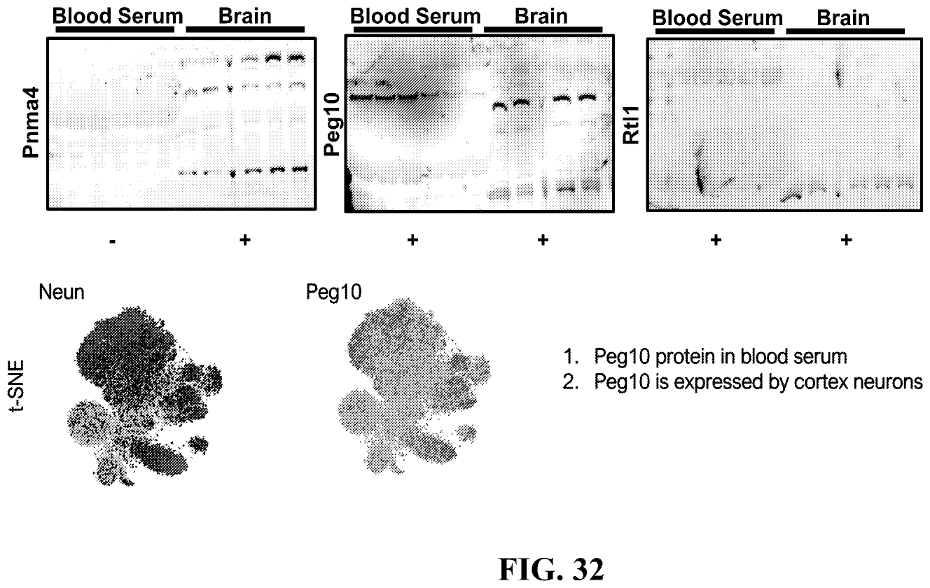

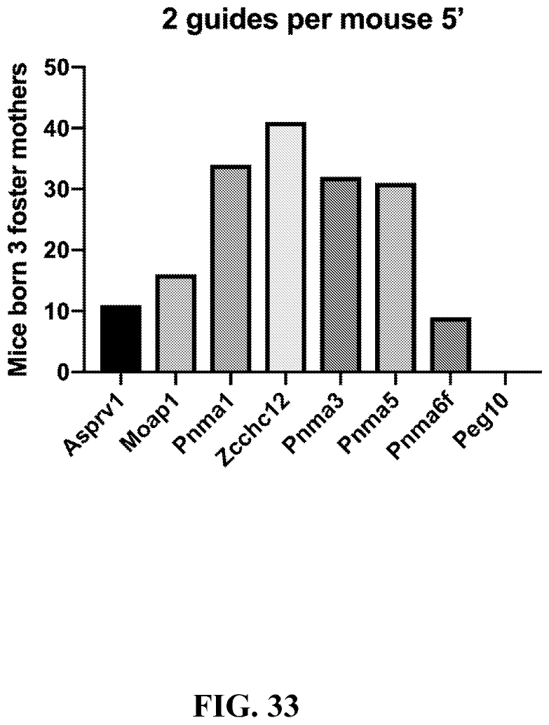

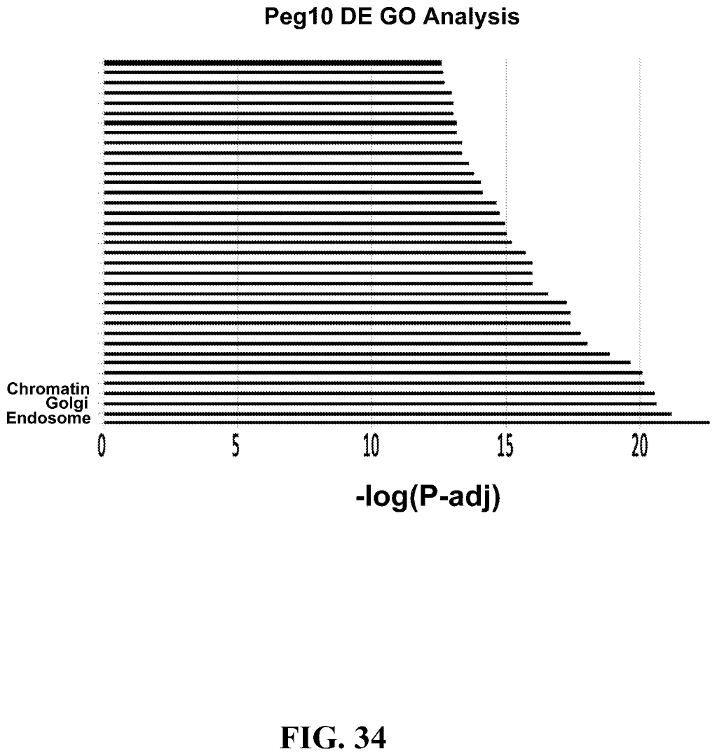

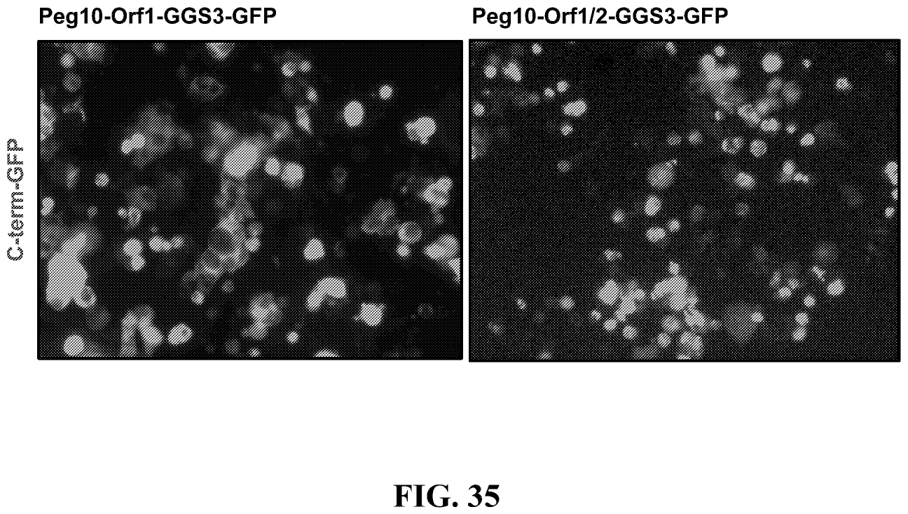

Applicants next determined that PEG10 can be found in both the blood serum and the cortex neurons in the brain (FIG. 32). Consistent with previous reports, knockout mice lacking PEG10 show early embryonic lethality, indicating the importance of this gene in embryonic development (FIG. 33). Gene ontology analysis of primary mouse neurons revealed three groups of differentially expressed genes: 1) genes involved in chromatin remodeling, 2) genes involved in the trans-golgi network and exocytosis, and 3) SNAREs and other genes coding for endosomal and transmembrane proteins.

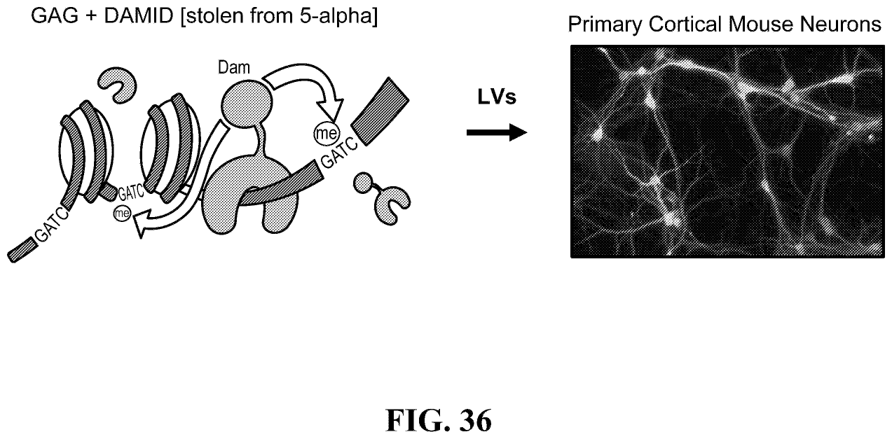

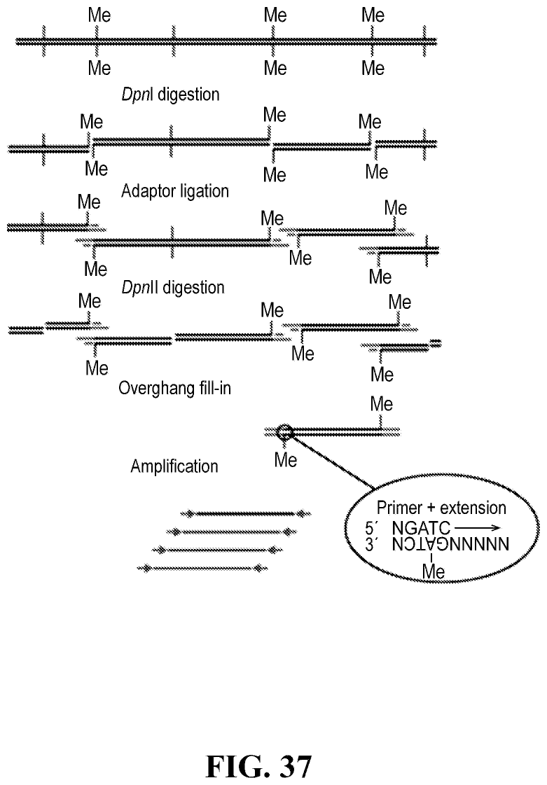

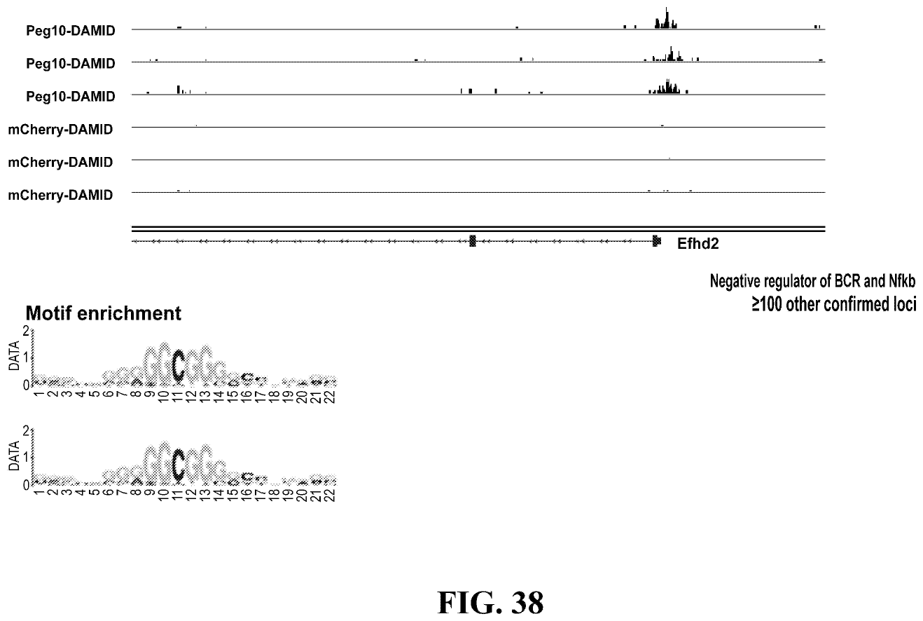

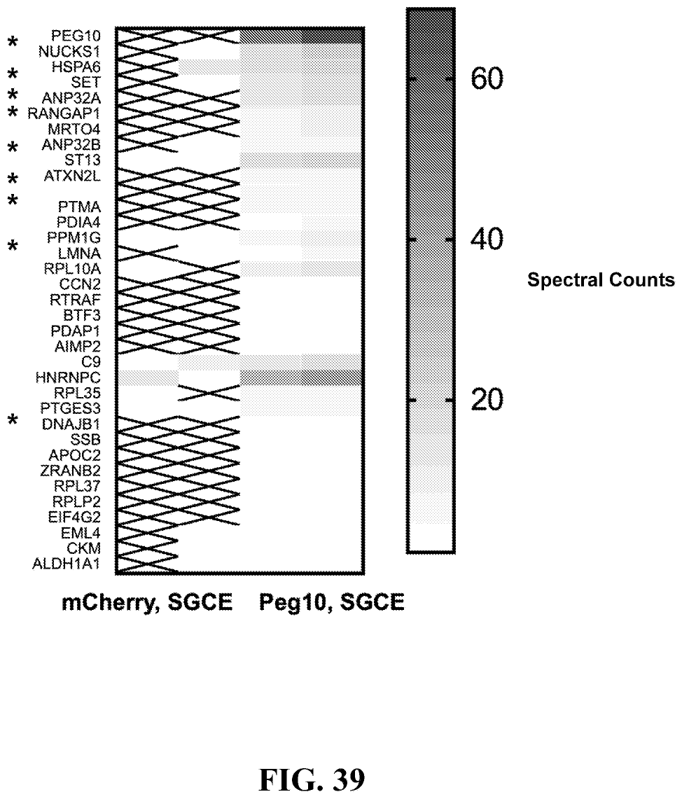

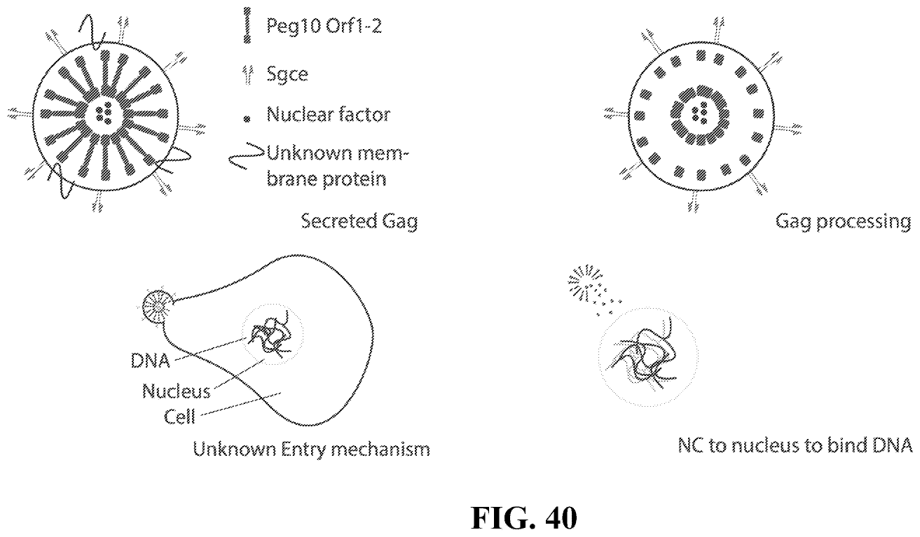

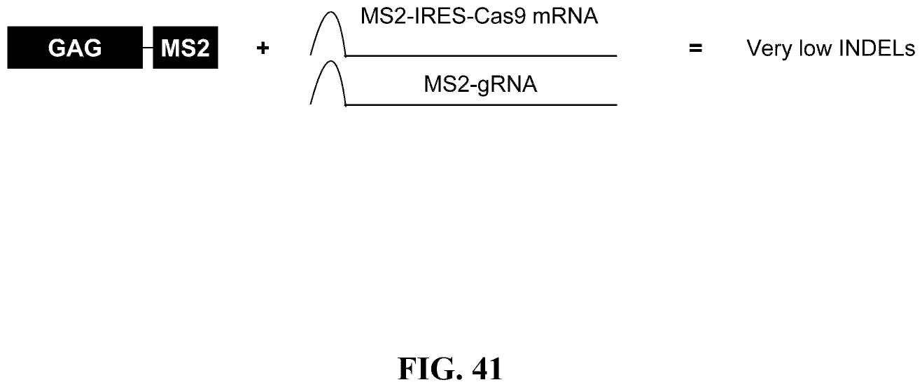

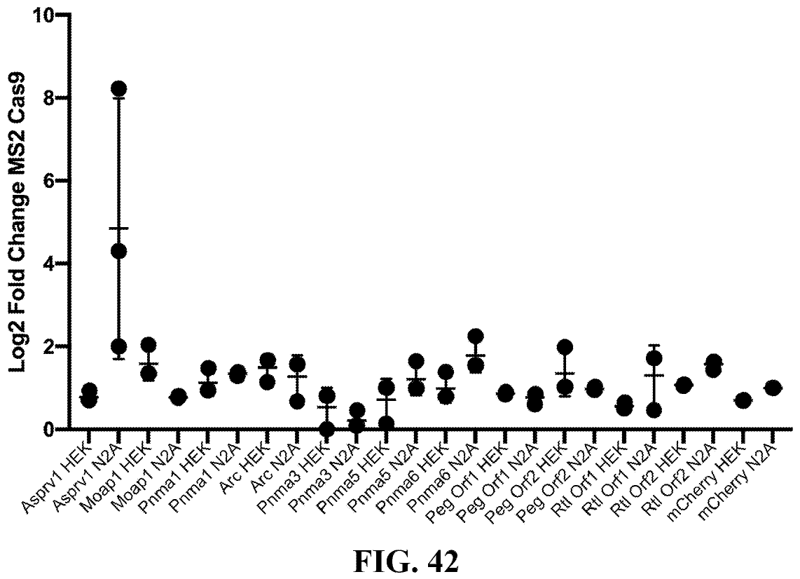

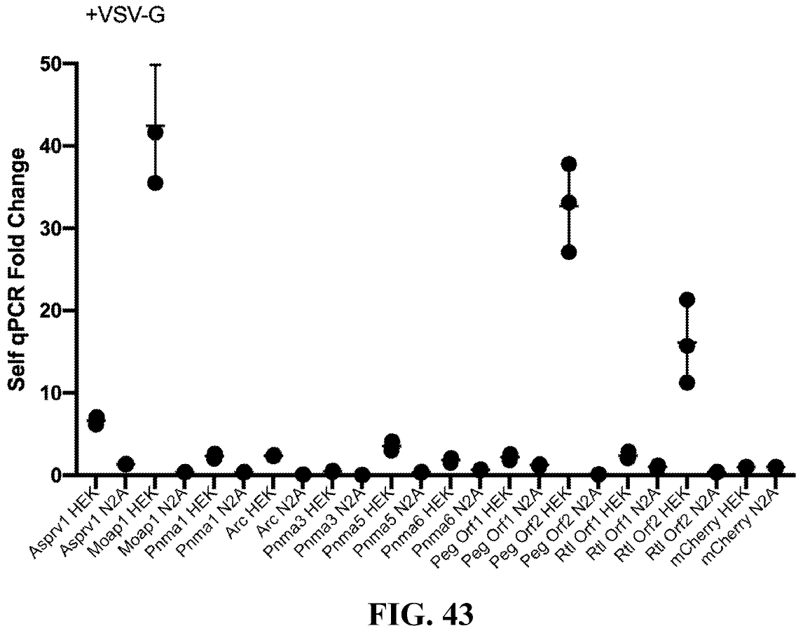

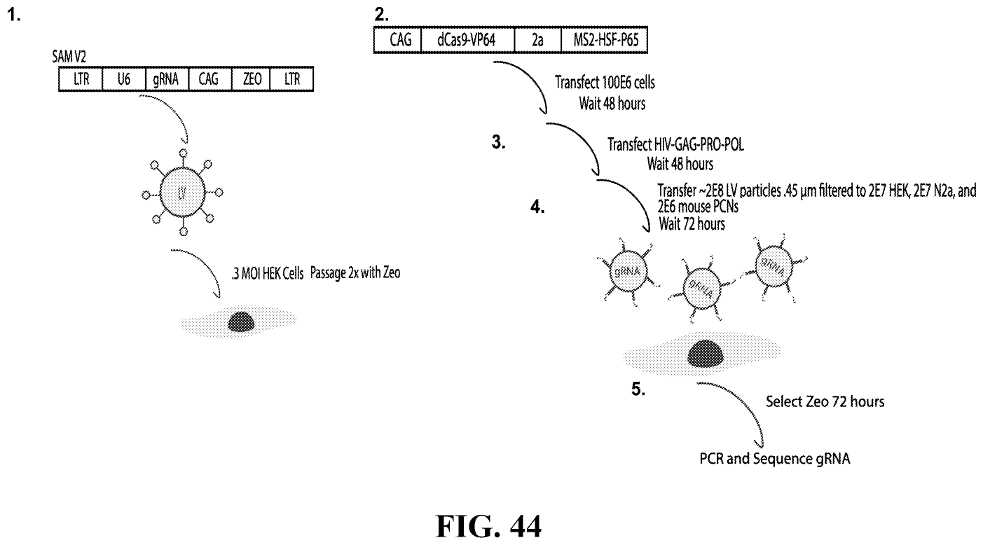

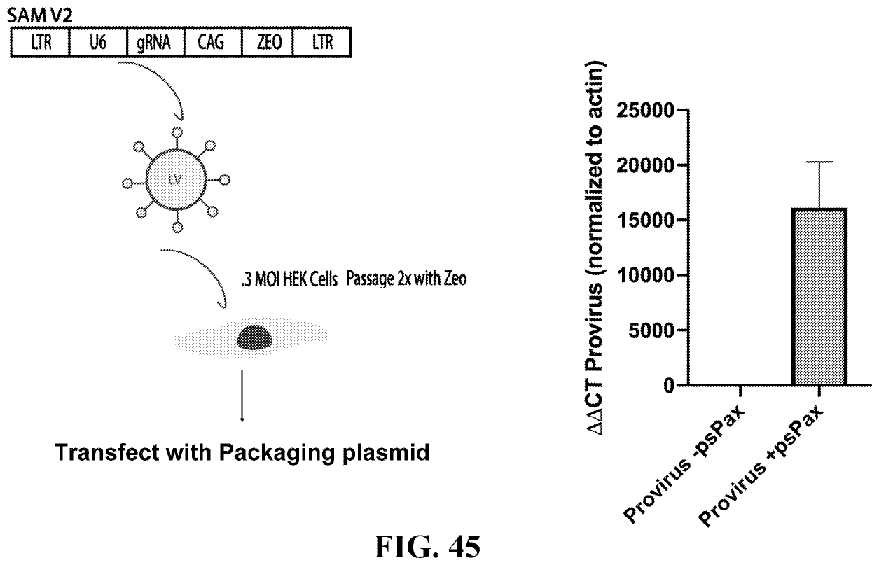

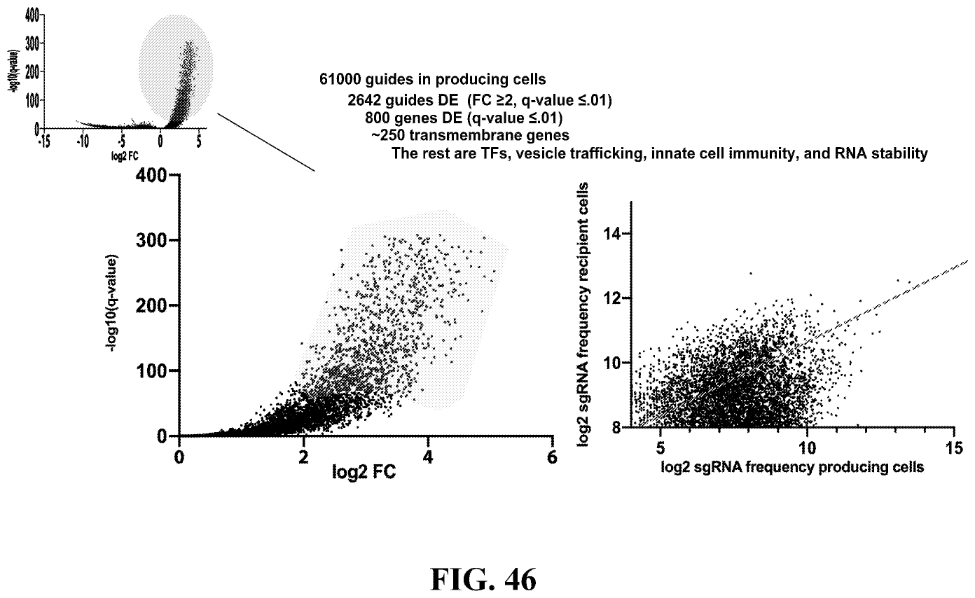

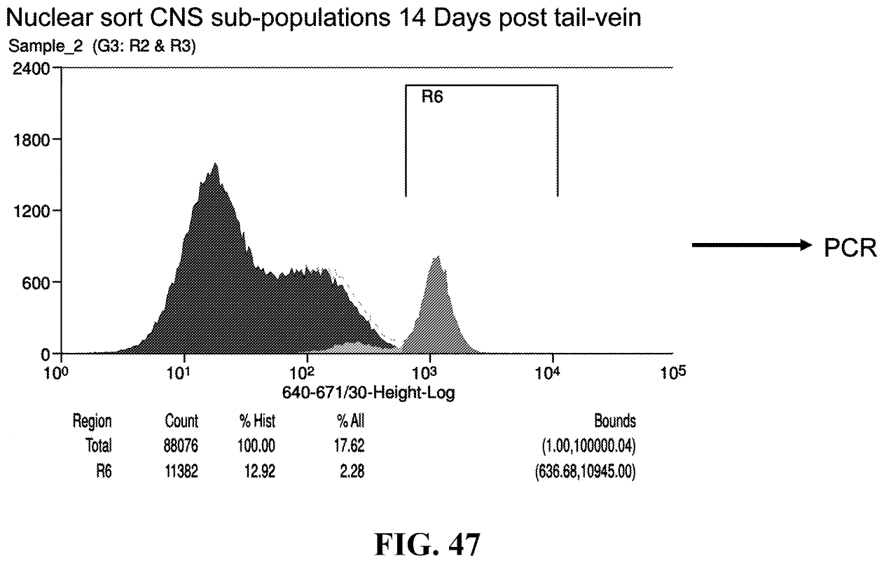

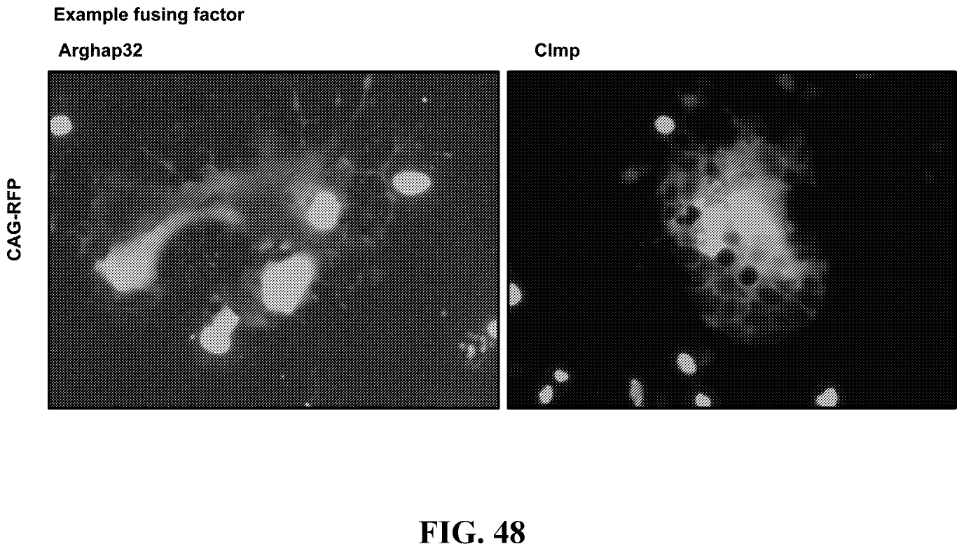

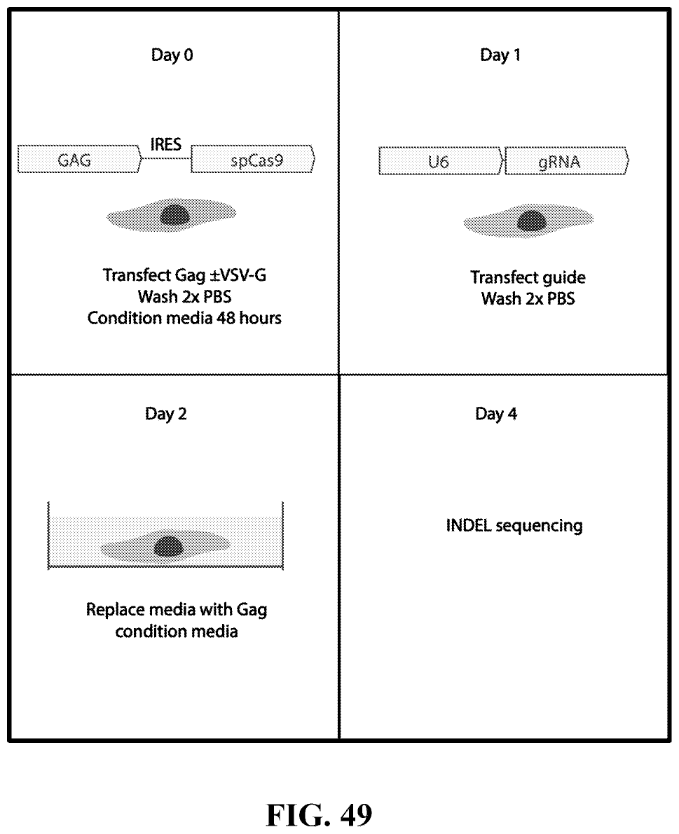

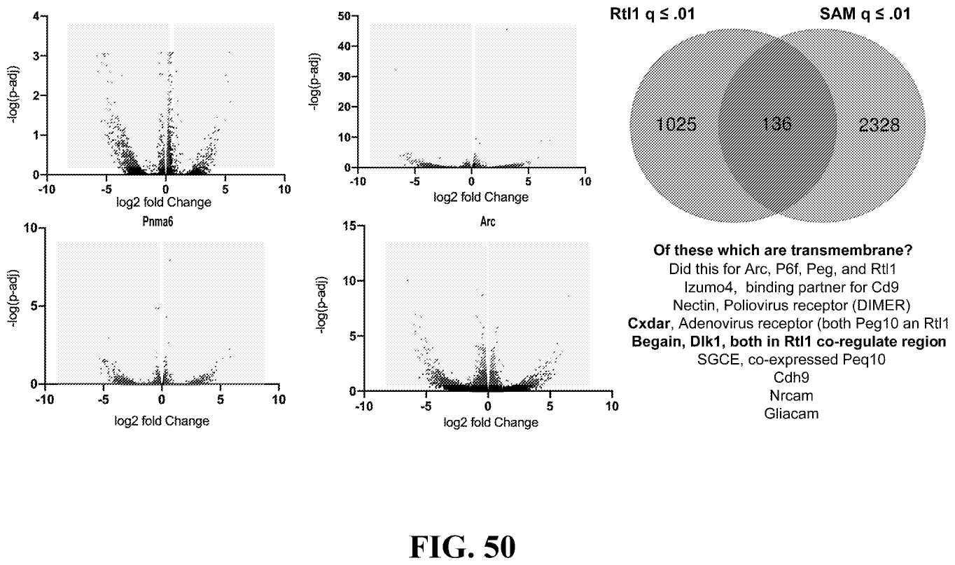

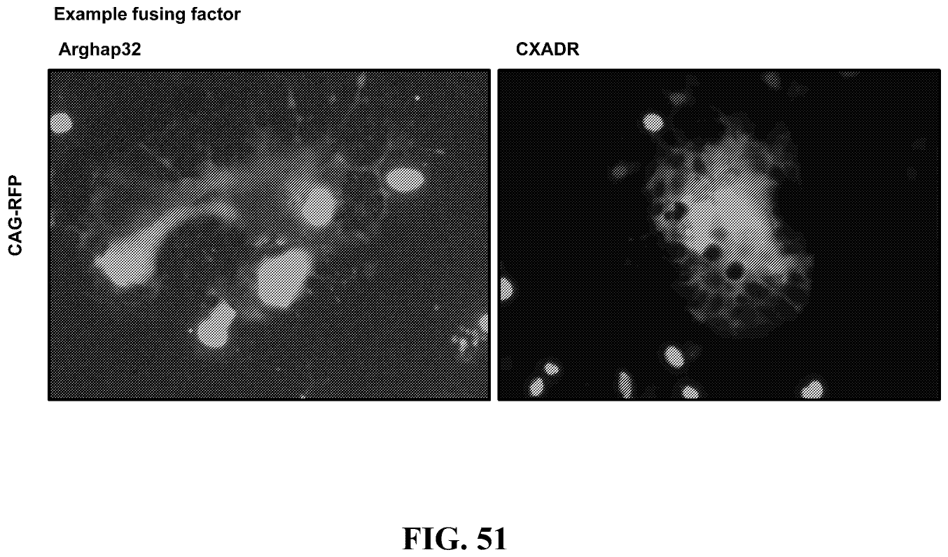

To figure out whether secreted GAGs are chromatin modifiers that bind DNA but not RNA, Applicants carried out DNA adenine methyltransferase identification (DamID), a protocol used to map the binding sites of DNA- and chromatin-binding protein in eukaryotes. DamID identifies binding sites by expressing the proposed DNA-binding protein as a fusion protein with DNA methyltransferase. Binding of the protein of interest to DNA localizes the methyltransferase in the region of the binding site. Adenosine methylation does not occur naturally in eukaryotes and therefore adenine methylation in any region can be concluded to have been caused by the fusion protein, implying the region is located near the binding site (FIG. 36). To carry out this protocol, Applicants digested the genome with DpnI, which cuts only methylated GATCs. Double-stranded adapters with a known sequence were then ligated to the ends generated by DpnI. Ligation products were digested using DpnII, which cuts non-methylated GATCs, ensuring that only fragments flanked by consecutive methylated GATCs were amplified in the subsequent PCR. A PCR with primers matching the adaptors was then carried out, leading to the specific amplification of genomic fragments flanked by methylated GATCs (FIG. 37). The data obtained by DamID mapping were then cross-referenced with ATAC-sequencing data (FIG. 38). Applicants overexpressed PEG10 and SGCE in N2A cells, ultracentrifuged the VLP fraction and analyzed precipitated proteins by mass-spectrometry. Various proteins, including RNA turnover factors, transcription factors, and chromatin remodelers were found to be enriched in this fraction (FIG. 39). Applicants concluded that PEG10 is efficiently secreted from cells and that it can mediate delivery of large macromolecules. Because PEG10 is found across the body, it likely binds DNA and may itself be what is delivered to cells, entering a cell and directly binding DNA (FIG. 40).

### Example 4

### Processing of PEG10 and Functional Properties of Processed Domains

The ability of PEG10 to form vesicles led to two central questions. 1) How is PEG10 processed, and, 2) what do each of the functional domains do? To answer the first question, Applicants overexpressed N- and C-terminal HA-tagged mouse PEG10 in HEK293FT cells, immunoprecipitated PEG10 using HA magnetic beads, and analyzed bands by Western blotting. Corresponding commassie-dyed bands were analyzed by mass-spectrometry. Results showed that the protein is cleaved into all the respective predicted domains (FIGS. 56, 57A-57F, 58A, and 58B).

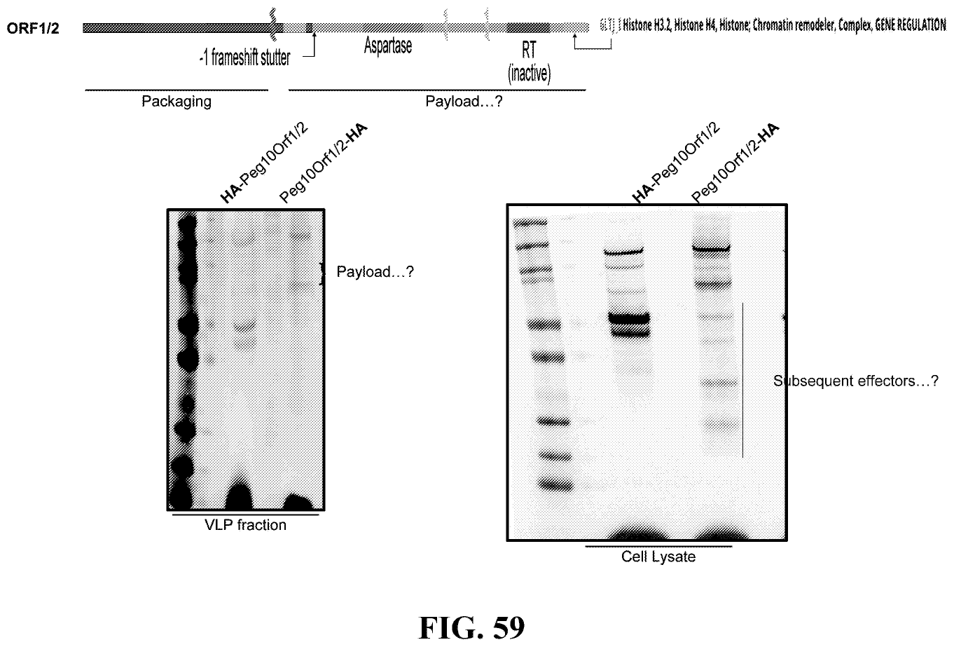

To answer the second question, Applicants compared PEG10 to a previously-identified protein known as MYEF, a DNA-binding protein that binds a very specific 10-basepair sequence in a 3× repeat (shown on the right side of FIG. 59). Applicants determined that PEG10 binds the exact same sequence, so they attempted to package particles that express the DNA sequence. When PEG10 was overexpressed with a plasmid DNA containing this sequence, Applicants noted that PEG10 preferentially packages and encapsulates that 10-basepair DNA sequence and secretes the plasmid carrying the sequence.

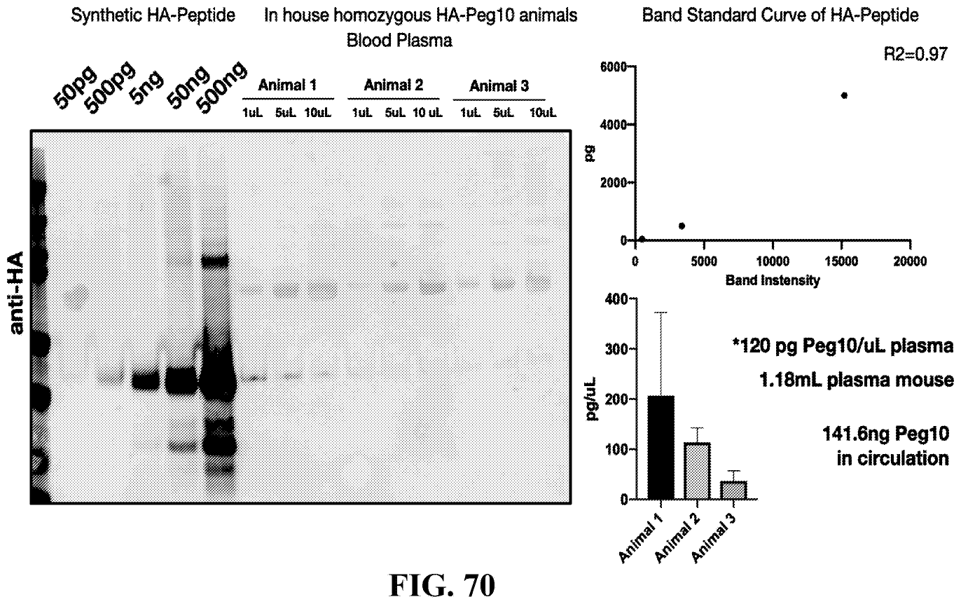

To quantify how much PEG10 circulates in the blood, Applicants engineered mice with a PEG10 antibody receptor tag and determined that PEG10 is expressed at about 120 pg/μL of blood plasma in mice (FIG. 70).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered delivery system comprising one or more polynucleotides encoding one or more endogenous retroviral elements for forming a delivery vesicle and one or more capture moieties for packaging a cargo within the delivery vesicle.

2. The system of claim 1, wherein the one or more endogenous retroviral elements for forming a delivery vesicle comprises two or more of a retroviral gag protein, a retroviral envelope protein, a retroviral reverse transcriptase or a combination thereof.

3. The system of claim 2, wherein the retroviral gag protein is endogenous.

4. The system of claim 2, wherein the retroviral envelope protein is endogenous.

5. The system of claim 2, wherein the retroviral gag protein and the retroviral envelope protein are both endogenous.

6. The system of claim 2 or 3, wherein the retroviral gag protein contains the NC and MA domains.

7. The system of any of claims 2 to 6, wherein the retroviral gag protein is a gag-homology protein.

8. The system of claim 7, wherein the gag-homology protein is Arc1, Asprv1, PNMA1, PNMA3, PNMA4, PNMA5, PNMA6, PNMA7, PEG10, RTL1, MOAP1, or ZCCHC12.

9. The system of claim 8, wherein the gag-homology protein is PNMA4, PEG10, or RTL1.

10. The system of claim 9, wherein the gag-homology protein is PEG10.

11. The system of any of claims 2 to 10, wherein the envelope protein is from a Gammaretrovirus or a Deltaretrovirus.

12. The system of any of claims 2 to 11, wherein the envelope protein is selected from envH1, envH2, envH3, envK1, envK2_1, envK2_2, envK3, envK4, envK5, envK6, envT, envW, envW1, envfrd, envR(b), envR, envF(c)2, or envF(c)1.

13. The system of any of claims 2 to 12, wherein the envelope protein comprises a cargo-binding domain.

14. The system of claim 13, wherein the cargo-binding domain is a hairpin loop-binding element.

15. The system of claim 13, wherein the hairpin loop-binding element is an MS2 aptamer.

16. The system of any of claims 1 to 15, wherein the delivery system elicits a poor immune response.

17. The system of any of claims 1 to 16, wherein the cargo comprises nucleic acids, proteins, a complex thereof, or a combination thereof.

18. The system of any of claims 1 to 17, wherein the cargo is linked to one or more envelope proteins by a linker.

19. The system of claim 18, wherein the linker is a glycine-serine linker.

20. The system of claim 19, wherein the glycine-serine linker is (GGS)3.

21. The system of claim 17, wherein the cargo comprises a ribonucleoprotein.

22. The system of claim 17, wherein the nucleic acid is DNA.

23. The system of any of claims 1 to 22, wherein the cargo comprises a genetic modulating agent.

24. The system of claim 23, wherein the genetic modulating agent comprises one or more components of a gene editing system and/or polynucleotides encoding thereof.

25. The system of claim 24, wherein the gene editing system is a CRISPR-Cas system.

26. The system of claim 25, wherein the CRISPR-Cas system is a Type II, Type V, or Type VI CRISPR-Cas system.

27. The system of claim 26, wherein the Type II CRISPR-Cas system comprises CRISPR-Cas9.

28. The system of claim 27, wherein the Type V CRISPR-Cas system comprises CRISPR-Cas12.

29. The system of claim 26, wherein the Type VI CRISPR-Cas system comprises CRISPR-Cas13.

30. The system of claim 25, wherein a Cas protein of the CRISPR-Cas system is modified to bind to a binding domain of the envelope protein.

31. The system of claim 25, wherein a guide molecule of the CRISPR-Cas system is modified to bind to a binding domain of the envelope protein.

32. The system of claim 30, wherein the modification comprises incorporation of a hairpin loop that binds to a hairpin-binding element on the envelope protein.

33. The system of claim 32, wherein the hairpin loop is recognized by the MS2 aptamer.

34. The system of any of claims 1 to 33, wherein the system further comprises a reverse transcriptase.

35. The system of any of claims 1 to 34, wherein the one or more capture moieties comprise DNA-binding moieties, RNA-binding moieties, protein-binding moieties, or a combination thereof.

36. The system of any of claims 1 to 35, wherein the delivery vesicle is a virus-like particle.

37. The system of any of claims 1 to 36, further comprising a targeting moiety, wherein the targeting moiety is capable of specifically binding to a target cell.

38. The system of claim 37, wherein the targeting moiety comprises a membrane fusion protein.

39. The system of claim 38, wherein the membrane fusion protein is the G envelope protein of vesicular stomatitis virus (VSV-G).

40. The system of claim 38, wherein the membrane fusion protein is SGCE.

41. The system of claim 37, wherein the target cell is a mammalian cell.

42. The system of claim 41, wherein the mammalian cell is a cancer cell.

43. The system of claim 42, wherein the mammalian cell is infected with a pathogen.

44. The system of claim 43, wherein the pathogen is a virus.

45. A delivery vesicle comprising one or more components encoded in the one or more polynucleotides in the engineered delivery system of any of the preceding claims.

46. The delivery vesicle of claim 45, wherein the one or more components comprises two or more of a retroviral gag protein, a retroviral envelope protein, a retroviral reverse transcriptase, or a combination thereof.

47. The delivery vesicle of claim 46, wherein the retroviral gag protein is a gag-homology protein selected from the group consisting of Arc1, Asprv1, PNMA1, PNMA3, PNMA4, PNMA5, PNMA6, PNMA7, PEG10, RTL1, MOAP1, and ZCCHC12.

48. The delivery vesicle of claim 47, wherein the gag-homology protein is PNMA4, PEG10, or RTL1.

49. The delivery system of claim 48, wherein the gag-homology protein is PEG10.

50. The delivery vesicle of any of claims 45 to 49, wherein the vesicle comprises a cell-specific targeting moiety.

51. The delivery vesicle of claim 50, wherein the cell-specific targeting moiety targets a mammalian cell.

52. The delivery vesicle of claim 51, wherein the cell-specific targeting moiety comprises a membrane fusion protein.

53. The delivery vesicle of claim 52, wherein the membrane fusion protein is VSV-G.

54. The delivery vesicle of claim 52, wherein the membrane fusion protein is SGCE.

55. The delivery vesicle of claim 51, wherein the mammalian cell is a cancer cell.

56. The delivery vesicle of claim 51, wherein the mammalian cell is infected with a pathogen.

57. The delivery vesicle of claim 56, wherein the pathogen is a virus.

58. A system for delivering a cargo to a target cell, comprising a delivery vesicle enclosing a cargo and an endogenous reverse transcriptase.

59. The system of claim 58, wherein the delivery vesicle is a virus-like particle.

60. The system of claim 58 or 59, wherein the delivery vesicle is comprised of a retroviral gag protein and a retroviral envelope protein.

61. The system of claim 60, wherein the retroviral gag protein originates from human endogenous retroviruses (HERVs).

62. The system of claim 61, wherein the retroviral gag protein is Arc1, Asprv1, PNMA1, PNMA3, PNMA4, PNMA5, PNMA6, PNMA7, PEG10, RTL1, MOAP1, or ZCCHC12.

63. The system of claim 62, wherein the retroviral gag protein is PNMA4, PEG10, or RTL1.

64. The system of claim 63, wherein the retroviral gag protein is PEG10.

65. The system of any of claim 58, wherein the retroviral envelope protein originates from HERVs.

66. The system of claim 58, wherein both the retroviral gag protein and the retroviral envelope protein originate from HERVs.

67. The system of any of claims 60 to 66, wherein the retroviral envelope protein comprises a cargo-binding domain.

68. The system of claim 67, wherein the cargo-binding domain is a hairpin loop-binding element.

69. The system of claim 68, wherein the hairpin loop-binding element is an MS2 aptamer.

70. The system of any of claims 58 to 69, wherein the cargo comprises nucleic acids, proteins, a complex thereof, or a combination thereof.

71. The system of claim 70, wherein the nucleic acid is DNA.

72. The system of claim 70, wherein the cargo comprises a ribonucleoprotein.

73. The system of any of claims 58 to 72, wherein the cargo comprises a genetic modulating agent.

74. The system of claim 73, wherein the genetic modulating agent comprises one or more components of a gene editing system and/or polynucleotides encoding thereof.

75. The system of claim 74, wherein the gene editing system is a CRISPR-Cas system.

76. The system of claim 75, wherein the CRISPR-Cas system is a Type II, Type V, or Type VI CRISPR-Cas system.

77. The system of claim 76, wherein the Type II CRISPR-Cas system comprises CRISPR-Cas9.

78. The system of claim 76, wherein the Type V CRISPR-Cas system comprises CRISPR-Cas12.

79. The system of claim 76, wherein the Type VI CRISPR-Cas system comprises CRISPR-Cas13.

80. The system of any of claims 58 to 79, wherein the cargo is linked to one or more envelope proteins by a linker.

81. The system of claim 80, wherein the linker is a glycine-serine linker.

82. The system of claim 81, wherein the glycine-serine linker is (GGS)3.

83. The system of claim 76, wherein a Cas protein of the CRISPR-Cas system is modified to bind to a binding domain of the envelope protein.

84. The system of claim 76, wherein a guide molecule of the CRISPR-Cas system is modified to bind to a binding domain of the envelope protein.

85. The system of claim 83, wherein the modification comprises incorporation of a hairpin loop that binds to a hairpin-binding element on the envelope protein.

86. The system of claim 85, wherein the hairpin loop is recognized by the MS2 aptamer.

87. The system of any of claims 58 to 86, further comprising a membrane fusion protein.

88. The system of claim 87, wherein the membrane fusion protein is VSV-G.

89. The system of claim 87, wherein the membrane fusion protein is SGCE.

90. The system of any of claims 58 to 89, wherein the target cell is a mammalian cell.

91. The system of claim 90, wherein the mammalian cell is a cancer cell.

92. The system of claim 90, wherein the mammalian cell is infected with a pathogen.

93. The system of claim 92, wherein the pathogen is a virus.

94. A method for loading cargo molecules into delivery vesicle systems comprising incubating a cargo molecule and the engineered delivery system of any of claims 1 to 43 with one or more bioreactors.

95. The method of claim 94, wherein the one of more bioreactors is a cell, a microorganism, or an acellular system.

96. A method for delivering cargo molecules comprising delivering the delivery vesicle of claims 45 to 57 to a target cell or cell population.

97. The method of claim 96, wherein delivery is in vivo.

98. The method of claim 96, wherein delivery is ex vivo.

99. The method of claim 96, wherein delivery is in vitro.

100. The method of any of claims 96 to 99, wherein the cargo comprises nucleic acids, proteins, a complex thereof, or a combination thereof.

101. The method of claim 100, wherein the nucleic acid is DNA.

102. The method of claim 100, wherein the cargo comprises a ribonucleoprotein.

103. The method of any of claims 100 to 102, wherein the cargo comprises a genetic modulating agent.

104. The method of any of claims 96 to 103, wherein delivery occurs across the blood-brain-barrier.
